(19) United States
(12) Patent Application Publication
(10) Pub. No.: US 2024/0336643 A1
(43) Pub. Date: Oct. 10, 2024

(54) BIFUNCTIONAL COMPOUNDS THAT DEGRADE ALK AND USES THEREOF

(71) Applicant: DANA-FARBER CANCER INSTITUTE, INC., Boston, MA (US)

(72) Inventors: Baishan Jiang, Watertown, MA (US); John M. Hatcher, Boston, MA (US); Lyn Howard Jones, Winchester, MA (US); Jianwei Che, Sharon, MA (US); Tinghu Zhang, Brookline, MA (US)

(73) Assignee: DANA-FARBER CANCER INSTITUTE, INC., Boston, MA (US)

(21) Appl. No.: 18/572,945

(22) PCT Filed: Jun. 27, 2022
(86) PCT No.: PCT/US2022/035121

§ 371 Date: Dec. 21, 2023

Related U.S. Application Data
(60) Provisional application No. 63/215,673, filed on Jun. 28, 2021.

(51) Int. Cl. C07F 9/6558; A61K 31/4545; A61K 31/496; A61K 31/506; A61K 31/675; A61K 47/55; A61P 35/00; C07D 401/14

(52) U.S. Cl. CPC C07F 9/65583; A61K 31/4545; A61K 31/496; A61K 31/506; A61K 31/675; A61K 47/55; A61P 35/00; C07D 401/14

(57) ABSTRACT

Disclosed are bifunctional compounds (degraders) that target ALK for degradation. Also disclosed are pharmaceutical compositions containing the degraders and methods of using the bifunctional compounds to treat diseases and disorders characterized or mediated by aberrant ALK activity.

### RELATED APPLICATIONS

This application claims the benefit of priority under 35 U.S.C. § 119(e) to U.S. Provisional Application No. 63/215,673, filed Jun. 28, 2021, which is incorporated herein by reference in its entirety.

### BACKGROUND OF THE INVENTION

Anaplastic lymphoma kinase (ALK) is a receptor tyrosine kinase that was first identified in a chromosomal translocation associated with anaplastic large cell lymphoma (ALCL), a subtype of T-cell non-Hodgkin's lymphoma (Chiarle et al., Nat. Rev. Cancer 8(1):11-23 (2008)). Chromosomal translocations involving the kinase domain of ALK are seen in many cancers. In addition to ALCL, ALK fusion proteins are seen in diffuse large B-cell lymphoma (DLBCL), inflammatory myofibroblastic tumor (IMT), breast cancer, colorectal cancer, esophageal squamous cell cancer (ESCC), renal cell cancer (RCC), and non-small-cell lung cancer (NSCLC) (Roskoski, Pharmacol. Res. 68(1):68-94 (2013)). ALK fusion partners drive dimerization of the ALK kinase domain, leading to autophosphorylation, which in turn causes the kinase to become constitutively active (Bayliss et al., Cell. Mol. Life Sci. 73(6):1209-1224 (2016)). Oncogenic ALK may also be expressed due to point mutations as is seen in neuroblastoma (NB), where germline mutations in ALK have been documented to drive the majority of hereditary NB cases (George et al., Nature 455(7215):975-978 (2008); Mosse et al., Nature 455(7215):930-935 (2008)). Constitutively active oncogenic ALK signals through multiple pathways, including PI3K/AKT, RAS/ERK, and JAK/STAT3, which leads to enhanced cell proliferation and survival (Palmer et al., Biochem. J. 420(3):345-361 (2009)).

ALK-rearranged NSCLC represents approximately 5% of all NSCLC and is a unique targetable molecular and clinical subset of NSCLC. Several studies have shown that NSCLC patients harboring ALK rearrangements are more likely to be non-smokers (Sasaki et al., Eur. J. Cancer 46(10):1773-1780 (2010); Mino-Kenudson et al., Clin. Cancer Res. 16(5):1561-1571 (2010)). Patients with tumors harboring such rearrangements are highly sensitive to ALK inhibitors (Arbour et al., Hematol. Oncol. Clin. North Am. 31(1):101-111 (2018)). Screening for ALK rearrangements is widely available throughout the United States and worldwide and is the standard of care for newly diagnosed advanced NSCLC patients (Camidge et al., Cancer 118(18):4486-4494 (2012); Kwak et al., New Engl. J. Med. 363(18):1693-1703 (2010)).

There are currently five FDA approved kinase inhibitors for the treatment of ALK-positive NSCLC, namely crizotinib, ceritinib (LDK378), alectinib, brigatinib and loratinib. ALK-positive tumors are highly sensitive to ALK inhibition, indicating that these tumors are addicted to ALK kinase activity. However, despite initial dramatic responses of variable median duration (e.g., 10.9 months for crizotinib, 25.7 months for alectinib), resistance to therapy typically develops (Peters et al., N. Engl. J. Med. 377:829-838 (2017); Soria et al., Lancet 389:917-929 (2017); Katayama et al., Sci. Trans. Med. 4(120):120ra17 (2012); Cooper et al., Ann. Pharmacother. 49:107-112 (2015); Sullivan et al., Ther. Adv. Med. Oncl. 8:32-47 (2016)). Next-generation ALK inhibitors such as lorlatinib (approved by the FDA in November 2018 for use in the treatment of lung cancer) have been able to successfully target resistant tumors and show improvements in potency and overall response rates relative to approved inhibitors.

However, resistance to these next-generation ALK inhibitors still arises in patients (Mologni et al., Transl. Lung Cancer Res. 4:5-7 (2015); Katayama et al., Clin. Cancer Res. 20:5686-5696 (2014); Qin et al., Targeted Oncology 12:709-718 (2017); Shaw et al., N. Engl. J. Med. 374:54-61 (2016)). The progression free survival period is currently at less than 12 months for patients that eventually acquire resistance (Mologni, Transl. Lung Cancer Res. 4(1):5-7 (2015); Katayama et al., Clin. Cancer Res. 20(22):5686-5696 (2014); Qin et al., Target. Oncol. 12(6):709-718 (2017); Shaw et al., New Engl. J. Med. 374(1):54-61 (2016)). The three most prevalent resistance mechanisms are mutation in the ALK kinase domain, upregulation of ALK as a result of gene amplification or copy number gain, and/or activation of ALK-independent signal transduction pathways (Roskoski, Pharmacol. Res. 68(1):68-94 (2013)).

Therapeutic strategies that target ALK employing novel mechanisms of action may provide ways to further delay the emergence of resistance mutations.

### SUMMARY OF THE INVENTION

A first aspect of the present invention is directed to a bifunctional compound of formula (I),

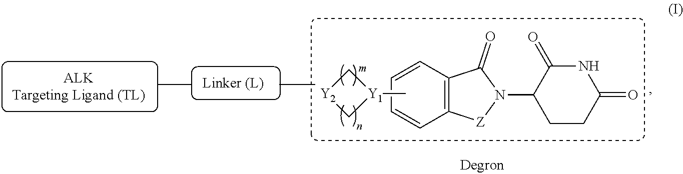

wherein,
the ALK Targeting Ligand is of Formula TL-1, TL-2, or TL-3:

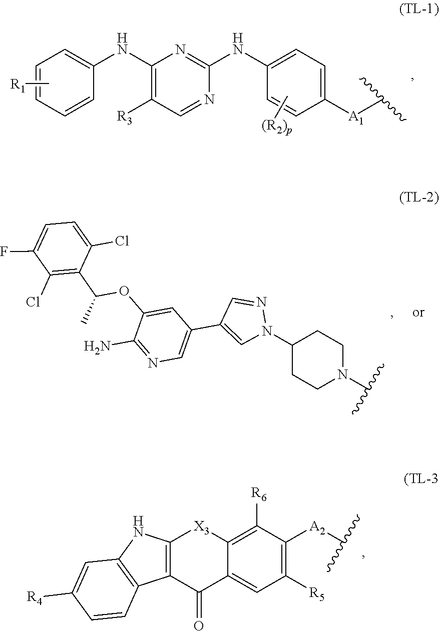

wherein A₁, A₂, X₃, Y₁, Y₂, R₁, R₂, R₃, R₄, R₅, R₆, R₇, Z, m, n, and p are as defined herein, and linker represents a moiety that covalently connects the degron and the targeting ligand, or a pharmaceutically acceptable salt or stereoisomer thereof.

Another aspect of the present invention is directed to a pharmaceutical composition containing a therapeutically effective amount of a bifunctional compound of formula (I) or a pharmaceutically acceptable salt or stereoisomer thereof, and a pharmaceutically acceptable carrier.

In another aspect of the present invention, methods of making the bifunctional compounds are provided.

A further aspect of the present invention is directed to a method of treating a disease or disorder involving (characterized or mediated by) aberrant ALK activity, that includes administering a therapeutically effective amount of a bifunctional compound of formula (I) or a pharmaceutically acceptable salt or stereoisomer thereof, to a subject in need thereof.

Without intending to be bound by any particular theory of operation, bifunctional compounds of formula I (also referred to herein as PROTACs or degraders) are believed to promote the degradation of ALK via cells' Ubiquitin/Proteasome System, whose function is to routinely identify and remove damaged proteins. After destruction of an ALK protein molecule, the degrader is released and continues to be active. Therefore, by engaging and exploiting the body's own natural protein disposal system, bifunctional compounds of the present invention may represent a potential improvement over current small molecule inhibitors of ALK. Therefore, effective intracellular concentrations of the degraders may be significantly lower than for small molecule ALK inhibitors.

Accordingly, bifunctional compounds of the present invention may offer at least one additional advantage including improved pharmacodynamics effects. The degradation of ALK may decrease tyrosine kinase inhibitor resistance imparted by intrinsic scaffolding functions of kinases and may also decrease the likelihood of de novo resistance mutations to the degraders since efficient degradation of ALK may be achieved with targeting ligands that have relatively less affinity to ALK compared to known ALK inhibitors. Collectively, present bifunctional compounds may represent an advancement over known ALK inhibitors and may overcome one or more limitations regarding their use.

### BRIEF DESCRIPTION OF THE DRAWINGS

FIG. 1 shows the relative fold-change (FC) abundance of proteins in MOLT4 cells treated with 1 μM compound 1 for 5 hours.

FIG. 2 shows the degradation of FER in the colon cancer cell line HCT116 by compound 1 at the indicated concentrations.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O— alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Therefore, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom or heteroatom-containing group (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur and oxygen. In some embodiments, heterocyclyl includes 4- to 6-membered monocycles having one or more heteroatoms selected from nitrogen, sulfur and oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺C₁⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Therefore, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Therefore, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format. To the extent not disclosed otherwise for any particular group(s), representative examples of substituents may include alkyl (e.g., C₁-C₆, C₁-C₅, C₁-C₄, C₁-C₃, C₁-C₂, C₁), substituted alkyl (e.g., substituted C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., substituted C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., substituted C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., substituted C₂-C₆, C₂-C₅, C₂-C₄, C₂-C₃, C₂), cyclic (e.g., C₃-C₁₂, C₅-C₆), substituted cyclic (e.g., substituted C₃-C₁₂, C₅-C₆), carbocyclic (e.g., C₃-C₁₂, C₅-C₆), substituted carbocyclic (e.g., substituted C₃-C₁₂, C₅-C₆), heterocyclic (e.g., 3-12 membered, 5-6 membered), substituted heterocyclic (e.g., substituted 3-12 membered, 5-6 membered), aryl (e.g., benzyl and phenyl), substituted aryl (e.g., substituted benzyl or substituted phenyl), heteroaryl (e.g., pyridyl or pyrimidyl), substituted heteroaryl (e.g., substituted pyridyl or substituted pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., substituted C₆-C₁₂, C₆), alkylthio (e.g., C₁-C₆), substituted alkylthio (e.g., substituted C₁-C₆), arylthio (e.g., C₆-C₁₂, C₆), substituted arylthio (e.g., substituted C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

As used herein, the phrase “optionally substituted with one or more halogen(s)” or “optionally substituted with C₆-C₁₀ aryl group(s)”, means at least one or more of said functional group provided that such substitution is in accordance with permitted valence of the substituted atom and the substituent.

The term “binding” as it relates to interaction between the targeting ligand and the targeted protein which is ALK, typically refers to an inter-molecular interaction that may be preferential (also referred to herein as “selective”) in that degradation of other proteins present in the cell is less and in some cases substantially less or even functionally insignificant. Present bifunctional compounds preferentially bind and recruit ALK for targeted degradation, including mutant forms thereof (e.g., EML4-ALK including the G1202R and L1196M mutants, and NPM-ALK) that manifest themselves in pathological states.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bifunctional compounds of the present invention have a structure represented by formula (I):

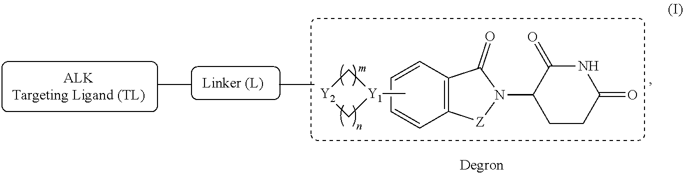

### ALK Targeting Ligand is of Formula TL-1, TL-2, or TL-3:

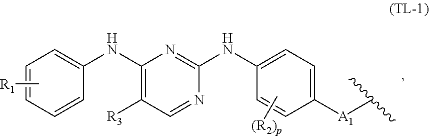

wherein:
A₁ is absent or

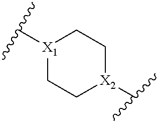

X₁ is CH or N;
X₂ is CH or N;
R₁ is —P(O)(Me)₂, —SO₂iPr, or —C(O)NHMe;
each R₂ is independently C₁-C₃ alkyl or C₁-C₃ alkoxy;
R₃ is hydrogen, halo, CN, CF₃, or C₁-C₃ alkyl; and
p is 1 or 2;

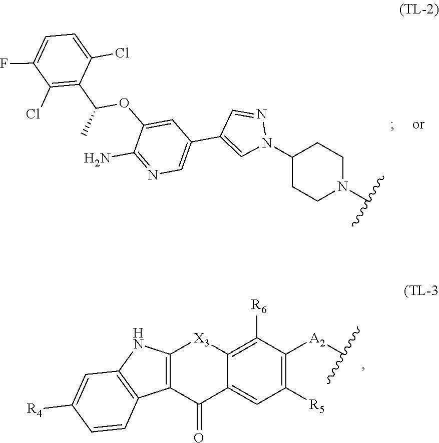

wherein:
A₂ is absent,

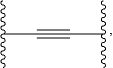 [SMILES: *C#C*]

5-membered heteroaryl, or 4- to 6-membered heterocyclyl;
X₃ is CMe₂ or NR₇;
   R₇ is C₁-C₈ alkyl or C₃-C₈ cycloalkyl; and
R₄, R₅, and R₆ are independently hydrogen, halo, CN, CF₃, C₁-C₃ alkyl, C₁-C₃ alkoxy, or C₃-C₆ cycloalkyl; and
Z is CH₂ or C═O;
Y₁ is N, CH, or COH;
Y₂ is N, CH, or cyclobutyl;
m is 1 or 2;
n is 1 or 2; and
the linker represents a moiety that connects covalently the degron and the targeting ligand, or a pharmaceutically acceptable salt or stereoisomer thereof.

### Targeting Ligands

In some embodiments, the ALK Targeting Ligand is of Formula TL-1.

Therefore, in some embodiments, the bifunctional compounds of the present invention have a structure represented by formula I-1:

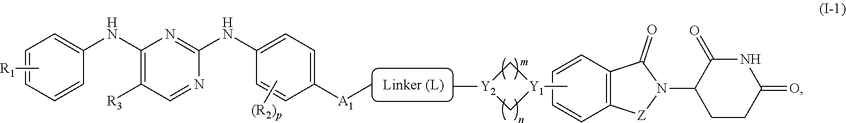

or a pharmaceutically acceptable salt or stereoisomer thereof.

In some embodiments of formula TL-1, A₁ is absent.

In some embodiments of formula TL-1, A₁ is

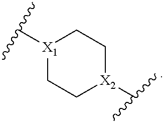

In some embodiments of formula TL-1, X₁ is CH.

In some embodiments of formula TL-1, X₁ is N.

In some embodiments of formula TL-1, X₂ is CH.

In some embodiments of formula TL-1, X₂ is N.

In some embodiments of formula TL-1, X₁ is CH and X₂ is N. In some embodiments of formula TL-1, X₁ is N and X₂ is CH. In some embodiments of formula TL-1, X₁ is N and X₂ is N.

In some embodiments of formula TL-1, R₁ is —P(O)(Me)₂.

In some embodiments of formula TL-1, R₁ is —SO₂iPr.

In some embodiments of formula TL-1, R₁ is —C(O)NHMe.

In some embodiments of formula TL-1, each R₂ is methyl, methoxy, ethoxy, or isopropoxy.

In some embodiments of formula TL-1, R₃ is halo. In some embodiments of formula TL-1, R₃ is chloro.

In some embodiments of formula TL-1, R₃ is CF₃.

In some embodiments of formula TL-1, p is 1.

In some embodiments of formula TL-1, p is 2.

In some embodiments of formula TL-1, p is 1 and R₂ is methoxy.

In some embodiments of formula TL-1, p is 2 and R₂ is methoxy and methyl.

In some embodiments of formula TL-1, p is 2 and R₂ is ethoxy and methyl.

In some embodiments of formula TL-1, p is 2 and R₂ is isopropoxy and methyl.

In some embodiments, the ALK Targeting Ligand is of Formula TL-1a to TL-1m:

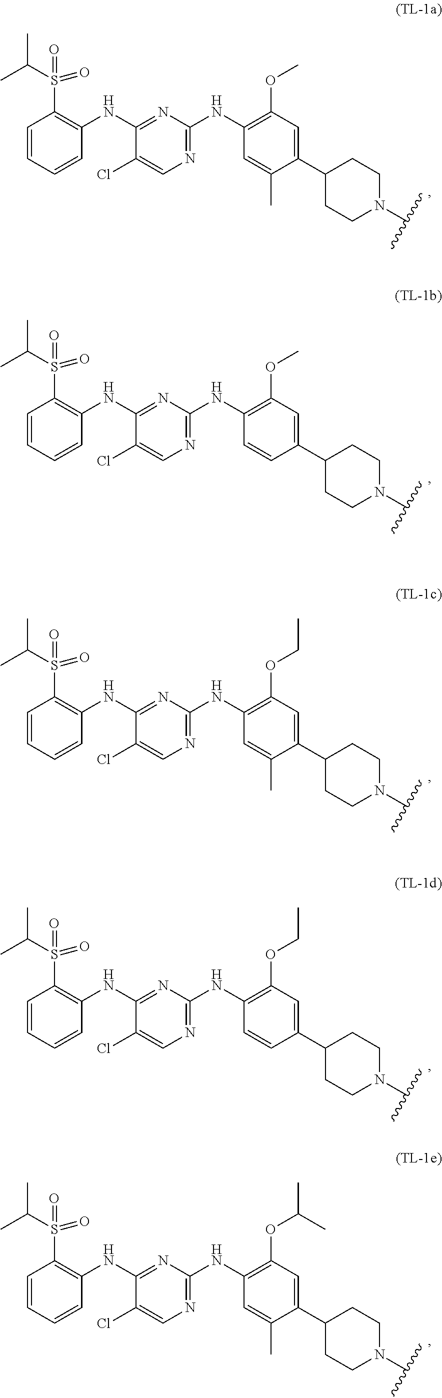 [SMILES: *N1CCC(c2cc(OC)c(Nc3ncc(Cl)c(Nc4ccccc4S(=O)(=O)C(C)C)n3)cc2C)CC1 ; *N1CCC(c2ccc(Nc3ncc(Cl)c(Nc4ccccc4S(=O)(=O)C(C)C)n3)c(OC)c2)CC1 ; *N1CCC(c2cc(OCC)c(Nc3ncc(Cl)c(Nc4ccccc4S(=O)(=O)C(C)C)n3)cc2C)CC1 ; *N1CCC(c2ccc(Nc3ncc(Cl)c(Nc4ccccc4S(=O)(=O)C(C)C)n3)c(OCC)c2)CC1 ; *N1CCC(c2cc(OC(C)C)c(Nc3ncc(Cl)c(Nc4ccccc4S(=O)(=O)C(C)C)n3)cc2C)CC1]

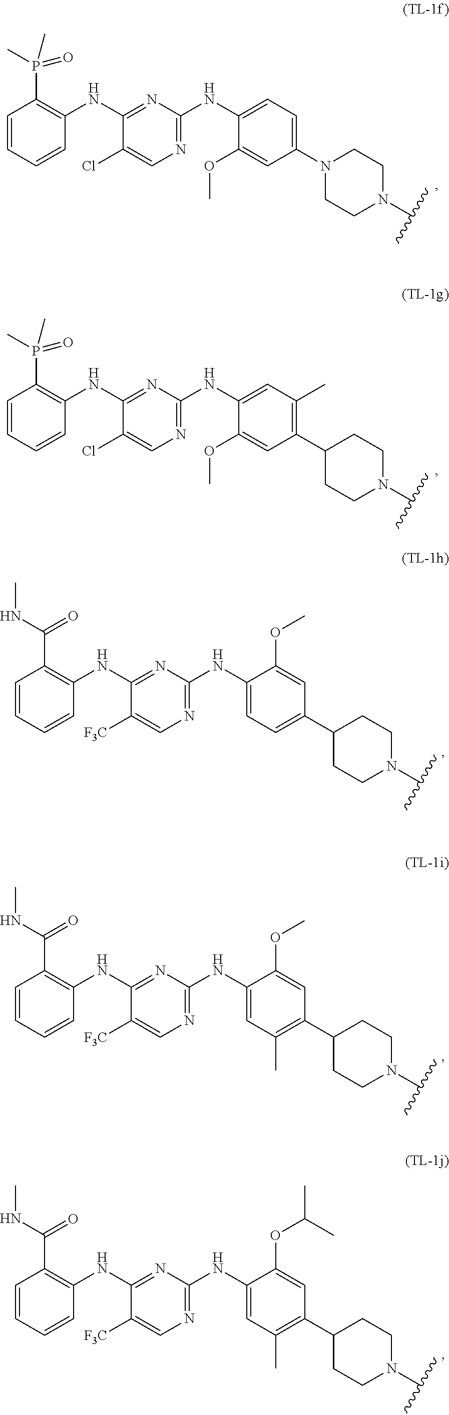 [SMILES: *N1CCN(c2ccc(Nc3ncc(Cl)c(Nc4ccccc4P(C)(C)=O)n3)c(OC)c2)CC1 ; *N1CCC(c2cc(OC)c(Nc3ncc(Cl)c(Nc4ccccc4P(C)(C)=O)n3)cc2C)CC1 ; *N1CCC(c2ccc(Nc3ncc(C(F)(F)F)c(Nc4ccccc4C(=O)NC)n3)c(OC)c2)CC1 ; *N1CCC(c2cc(OC)c(Nc3ncc(C(F)(F)F)c(Nc4ccccc4C(=O)NC)n3)cc2C)CC1 ; *N1CCC(c2cc(OC(C)C)c(Nc3ncc(C(F)(F)F)c(Nc4ccccc4C(=O)NC)n3)cc2C)CC1]

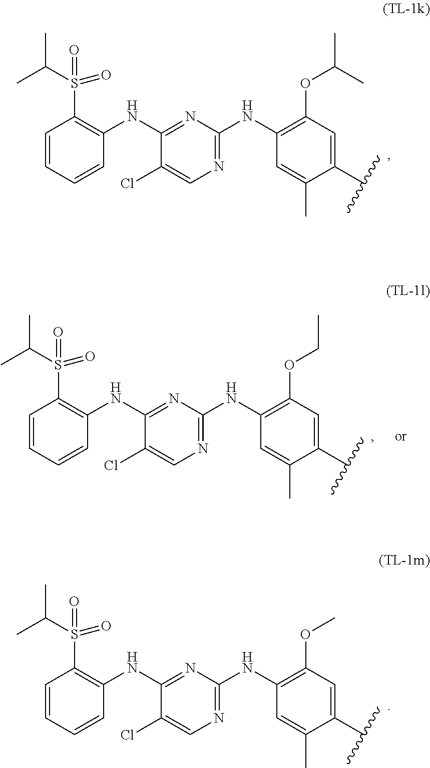

Therefore, in some embodiments, the bifunctional compounds of the present invention have a structure represented by formula I-1a to I-1m:

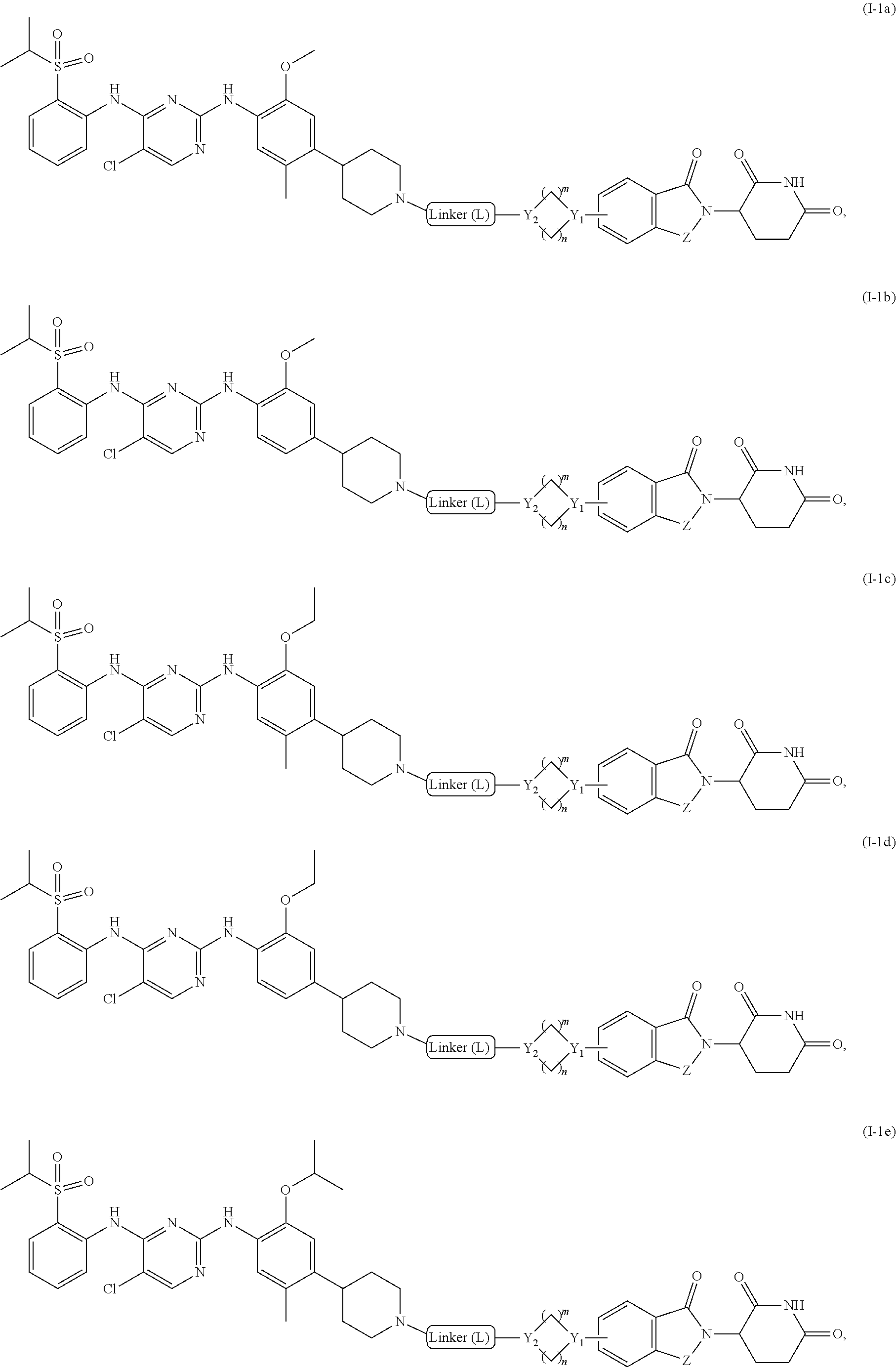

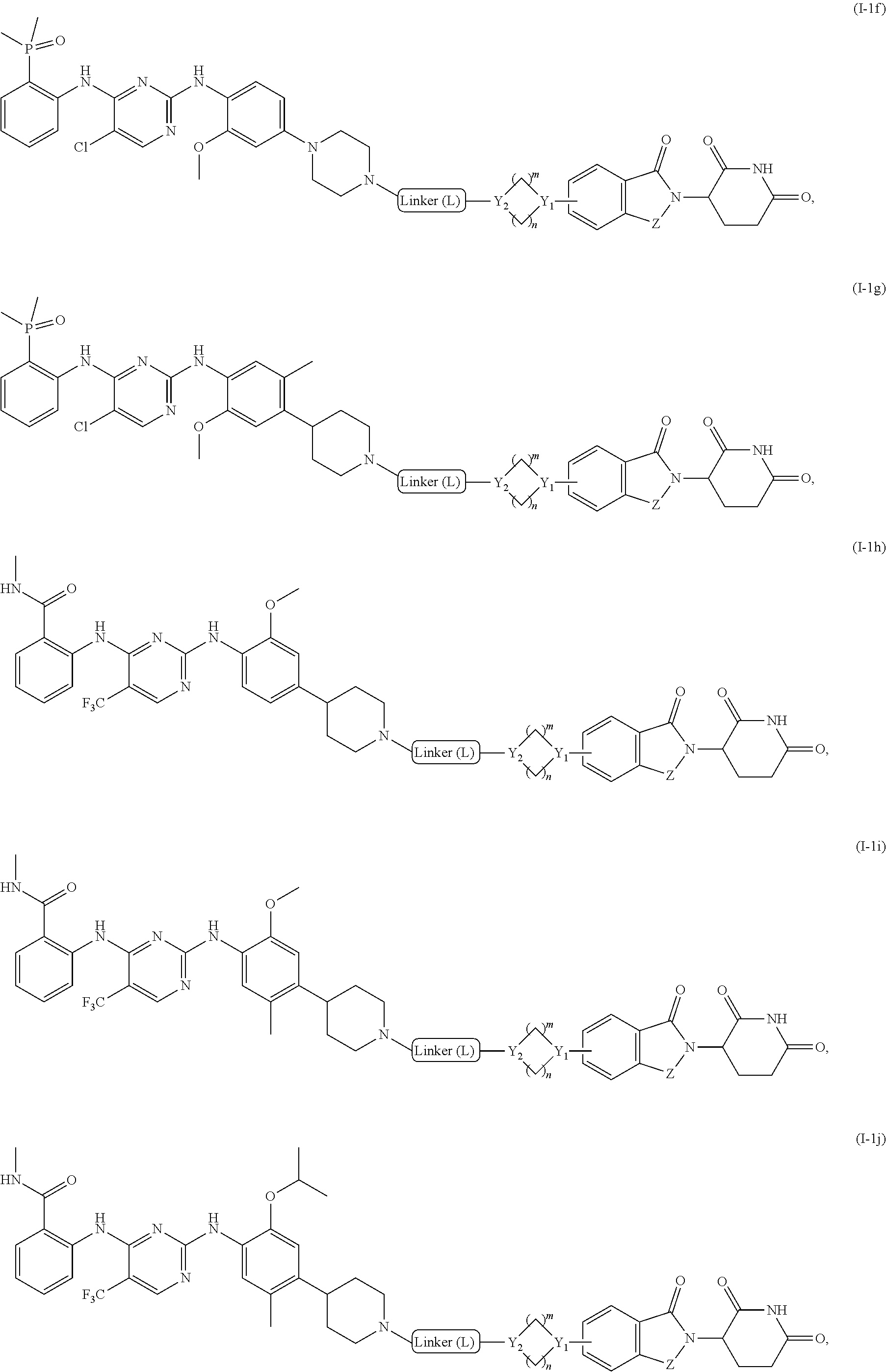

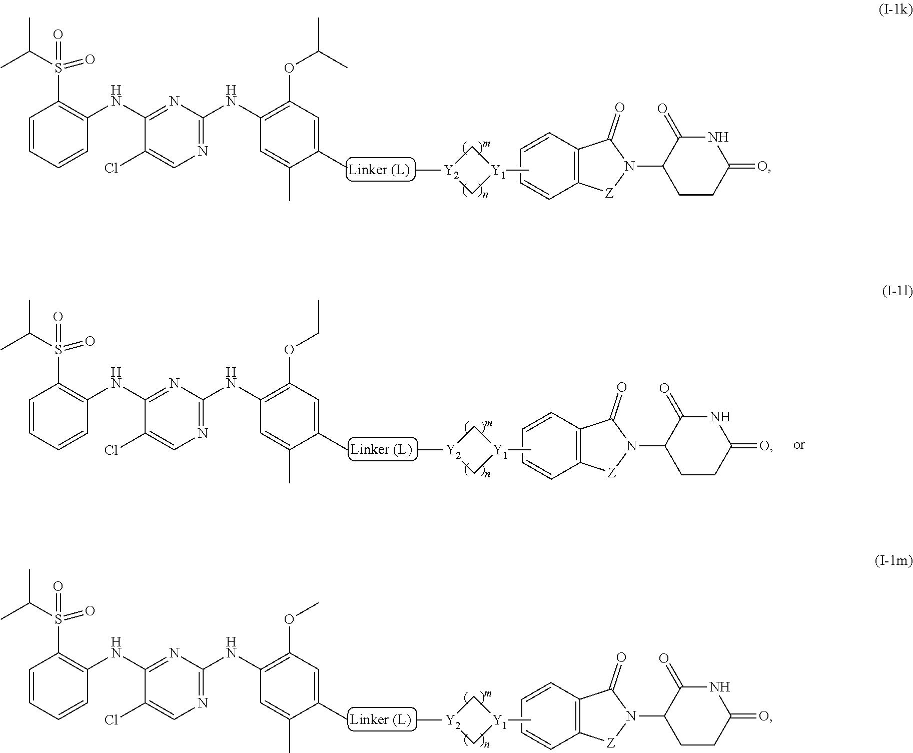

or a pharmaceutically acceptable salt or stereoisomer thereof.

In some embodiments, the ALK Targeting Ligand is of Formula TL-2.

Therefore, in some embodiments, the bifunctional compounds of the present invention have a structure represented by formula I-2:

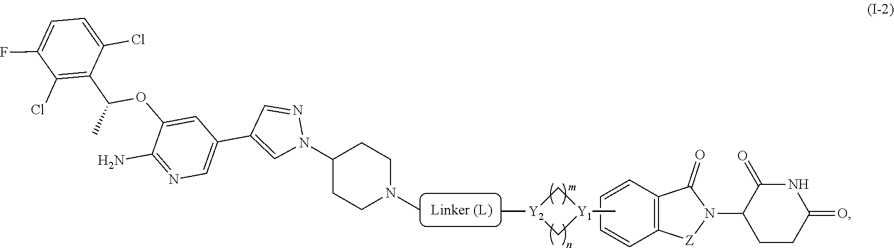

or a pharmaceutically acceptable salt or stereoisomer thereof.

In some embodiments, the ALK Targeting Ligand is of Formula TL-3.

Therefore, in some embodiments, the bifunctional compounds of the present invention have a structure represented by formula I-3:

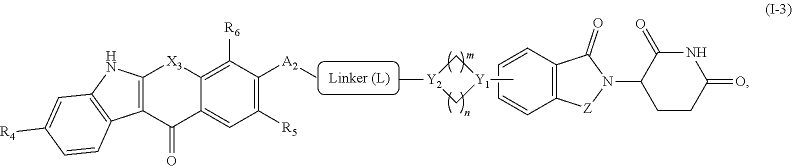

or a pharmaceutically acceptable salt or stereoisomer thereof.

In some embodiments of formula TL-3, A₂ is absent.

In some embodiments of formula TL-3, A₂ is

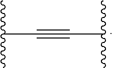 [SMILES: *C#C*]

In some embodiments of formula TL-3, A₂ is 5-membered heteroaryl, containing 1-2 heteroatoms selected from N and O. In some embodiments of formula TL-3, A₂ is pyrazole.

In some embodiments of formula TL-3, A₂ is 4- to 6-member heterocyclyl, containing 1-2 heteroatoms selected from N and O. In some embodiments of formula TL-3, A₂ is azetidine, piperidine, or piperazine.

In some embodiments of formula TL-3, X₃ is CMe₂.

In some embodiments of formula TL-3, R₄ is CN.

In some embodiments of formula TL-3, R₅ is ethyl.

In some embodiments of formula TL-3, R₆ is hydrogen.

In some embodiments, the ALK Targeting Ligand is of Formula TL-3a to TL-3i:

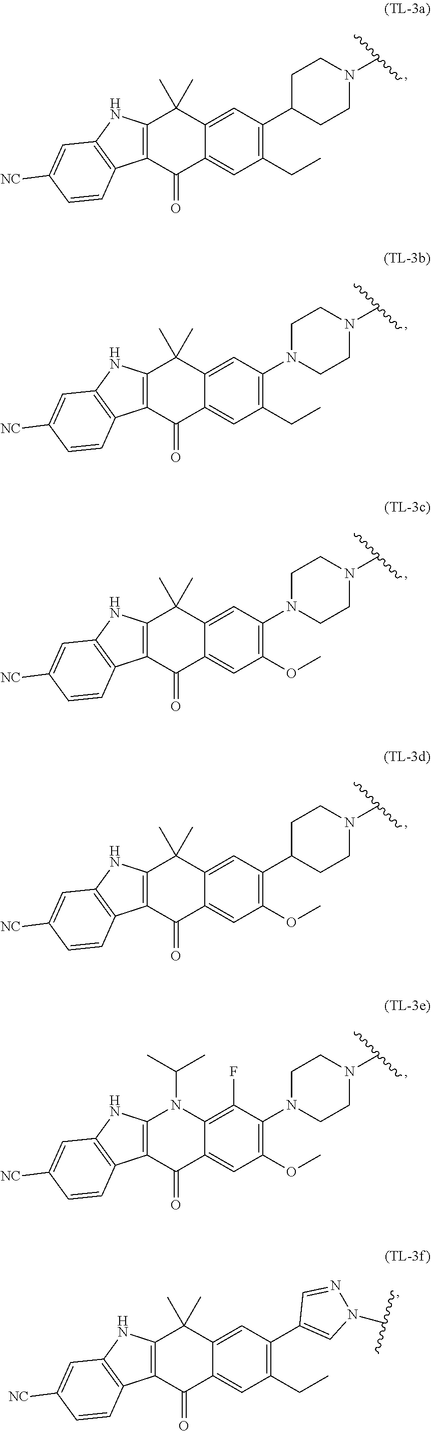 [SMILES: *N1CCC(c2cc3c(cc2CC)C(=O)c2c([nH]c4cc(C#N)ccc24)C3(C)C)CC1 ; *N1CCN(c2cc3c(cc2CC)C(=O)c2c([nH]c4cc(C#N)ccc24)C3(C)C)CC1 ; *N1CCN(c2cc3c(cc2OC)C(=O)c2c([nH]c4cc(C#N)ccc24)C3(C)C)CC1 ; *N1CCC(c2cc3c(cc2OC)C(=O)c2c([nH]c4cc(C#N)ccc24)C3(C)C)CC1 ; *N1CCN(c2c(OC)cc3c(=O)c4c5ccc(C#N)cc5[nH]c4n(C(C)C)c3c2F)CC1 ; *n1cc(-c2cc3c(cc2CC)C(=O)c2c([nH]c4cc(C#N)ccc24)C3(C)C)cn1]

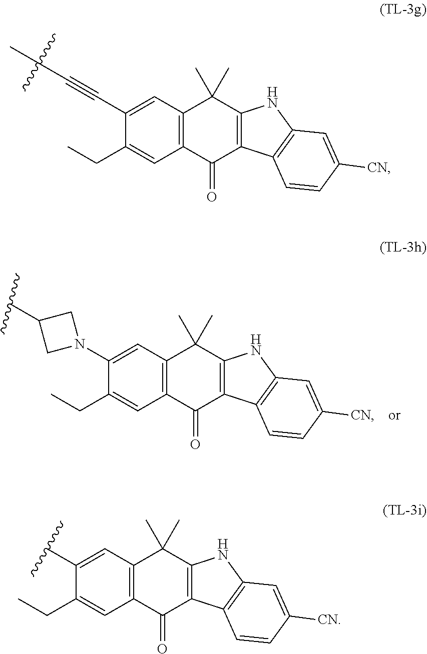 [SMILES: *C#Cc1cc2c(cc1CC)C(=O)c1c([nH]c3cc(C#N)ccc13)C2(C)C ; *C1CN(c2cc3c(cc2CC)C(=O)c2c([nH]c4cc(C#N)ccc24)C3(C)C)C1 ; *c1cc2c(cc1CC)C(=O)c1c([nH]c3cc(C#N)ccc13)C2(C)C]

Therefore, in some embodiments, the bifunctional compounds of the present invention have a structure represented by formula I-3a to I-3i:

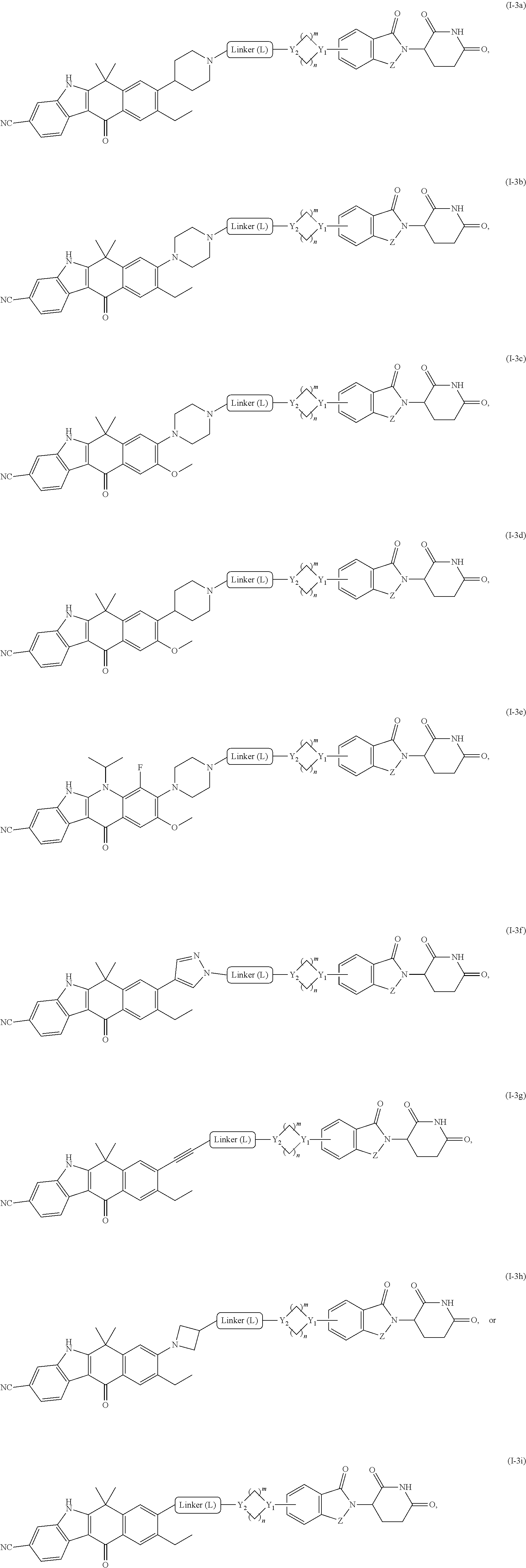

pharmaceutically acceptable salt or stereoisomer thereof.

### Linkers

The linker (“L”) provides a covalent attachment between the targeting ligand and the degron. The structure of linker may not be critical, provided it is substantially non-interfering with the activity of the ALK targeting ligand or the degron.

In some embodiments, the Linker is of Formula L0:

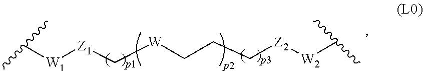

or stereoisomer thereof, wherein
p1 is an integer selected from 0 to 6;
p2 is an integer selected from 0 to 12;
p3 is an integer selected from 0 to 12;
each W is independently absent, CH₂, O, S, NR₁₀, or C(O)NH;
   each R₁₀ is independently hydrogen or C₁-C₆ alkyl;
W₁ and W₂ are independently absent, (CH₂)₁₋₃, O, or NH; and
Z₁ and Z₂ are independently absent, —O—, —S—, —N(R₁₀)—, —C≡C—, —C(O)—, —C(O)O—, —OC(O)—, —OC(O)O—, —C(NOR₁₀)—, —C(O)N(R₁₀)—, —C(O)N(R₁₀)C(O)—, —C(O)N(R₁₀)C(O)N(R₁₀)—, —N(R₁₀)C(O)—, —N(R₁₀)C(O)N(R₁₀)—, —N(R₁₀)C(O)O—, —OC(O)N(R₁₀)—, —C(NR₁₀)—, —N(R₁₀)C(NR₁₀)—, —C(NR₁₀)N(R₁₀)—, —N(R₁₀)C(NR₁₀)N(R₁₀)—, —OB(Me)O—, —S(O)₂—, —OS(O)—, —S(O)O—, —S(O)—, —OS(O)₂—, —S(O)₂O—, —N(R₁₀)S(O)₂—, —S(O)₂N(R₁₀)—, —N(R₁₀)S(O)—, —S(O)N(R₁₀)—, —N(R₁₀)S(O)₂N(R₁₀)—, —N(R₁₀)S(O)N(R₁₀)—, C₃-C₁₂ carbocyclyl, or 3- to 12-membered heterocyclyl;
wherein the Linker is covalently bonded to a Degron via the

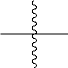

next to W₂, and covalently bonded to a Targeting Ligand via the

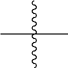

next to W₁, or the Linker is covalently bonded to a Degron via the

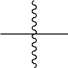

next to W₁, and covalently bonded to a Targeting Ligand via the

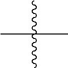

next to W₂.

In some embodiments, the linker includes an alkylene chain (e.g., having 2-20 alkylene units). In other embodiments, the linker may include an alkylene chain or a bivalent alkylene chain, either of which may be interrupted by, and/or terminate (at either or both termini) at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker includes an alkylene chain having 2-20 alkylene units. In some embodiments, the linker includes an alkylene chain having 3-12 alkylene units. In some embodiments, the linker includes an alkylene chain having 1-3 alkylene units. In some embodiments, the linker includes an alkylene chain having 1-3 alkylene units, optionally substituted with C(O). In some embodiments, the linker includes an alkylene chain having 1-2 alkylene units optionally substituted with C(O).

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of linkers that may be suitable for use in the present invention include alkylene chains:

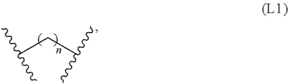

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

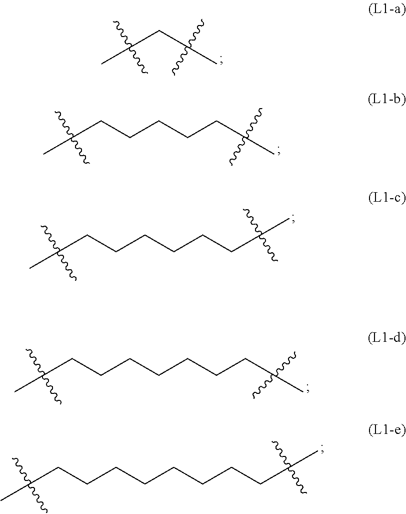

alkylene chains terminating in various functional groups (as described above), examples of which are as follows:

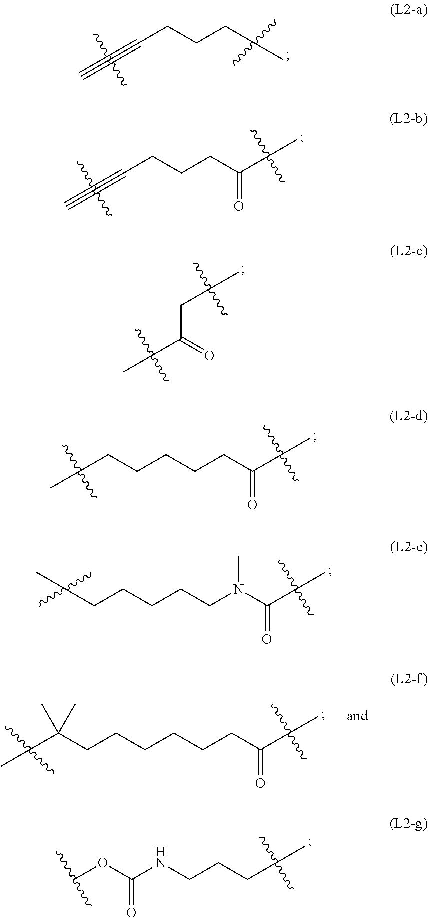

alkylene chains interrupted by various functional groups (as described above), examples of which are as follows:

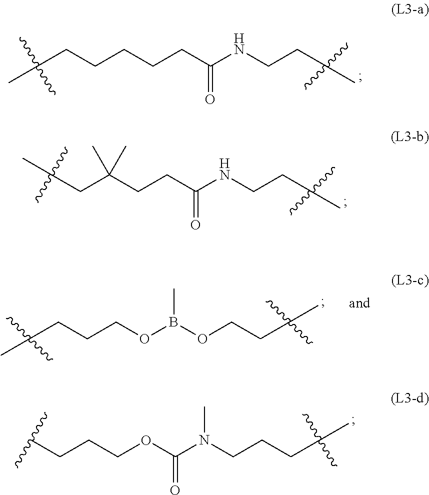 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted by or terminating with heterocyclene groups, e.g.,

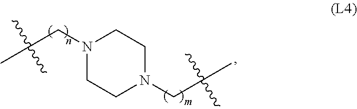

wherein m and n are independently integers of 0-10, examples of which include:

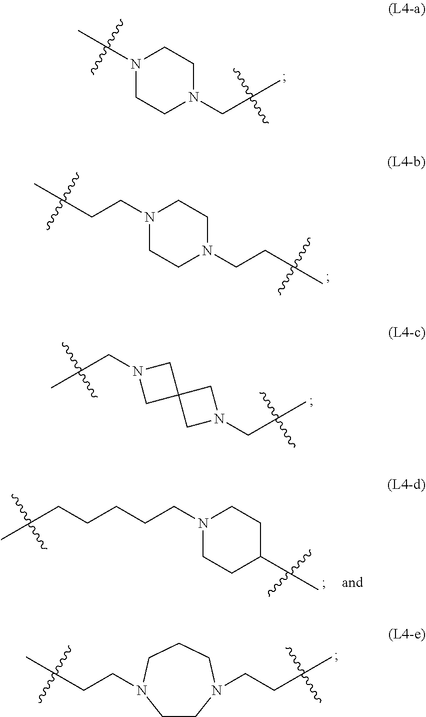 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

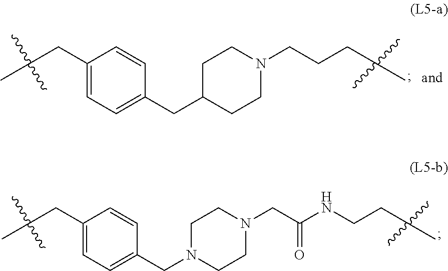 [SMILES: *CCCN1CCC(Cc2ccc(C*)cc2)CC1 ; *CCNC(=O)CN1CCN(Cc2ccc(C*)cc2)CC1]

alkylene chains interrupted by heterocyclene and aryl groups, and a heteroatom, examples of which include:

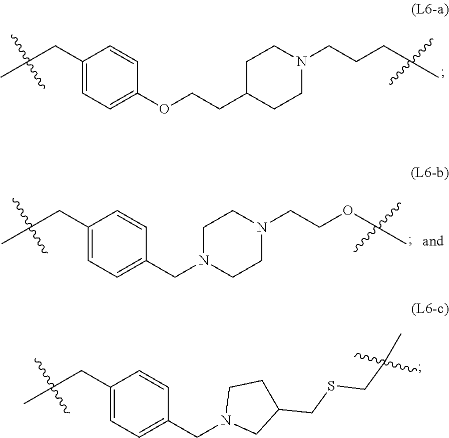 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and
alkylene chains interrupted by and/or terminating in a heteroatom such as N, O or B, e.g.,

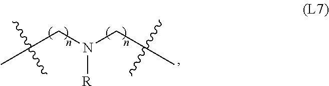

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C1 to C4 alkyl, an example of which is

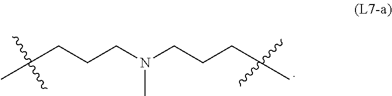 [SMILES: *CCCN(C)CCC*]

In some embodiments, the linker may include a polyethylene glycol (PEG) chain which may terminate at either or both termini with at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the linker includes a polyethylene glycol chain having 1-10 PEG units. In some embodiments, the linker includes a polyethylene glycol chain having 1-6 PEG units. In some embodiments, the linker includes a polyethylene glycol chain having 1-2 PEG units.

Examples of linkers that include a polyethylene glycol chain include:

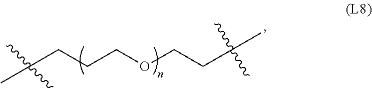

wherein n is an integer of 2-10, examples of which include:

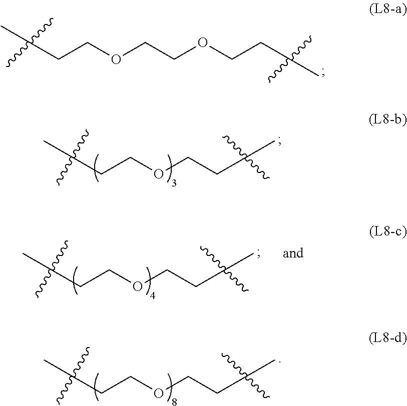

In some embodiments, the linker containing a polyethylene glycol chain may terminate in a functional group, examples of which are as follows:

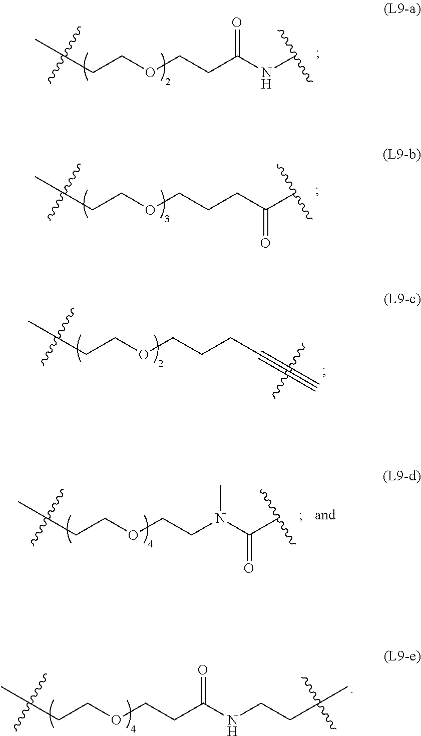

In some embodiments, the bifunctional compound of formula (I) includes a linker that is represented by any one of the following structures:

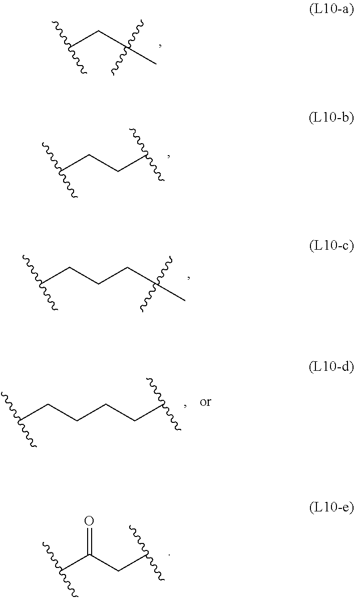

In some embodiments, the bifunctional compounds of the present invention are represented by any one of the following structures:

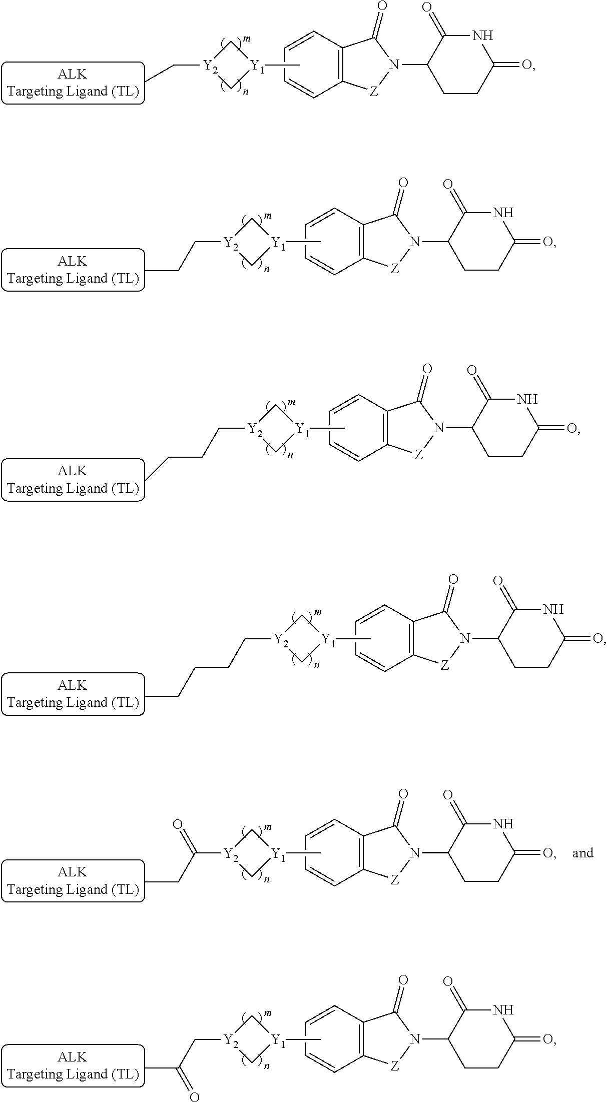

pharmaceutically acceptable salt or stereoisomer thereof.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

Degrons bind the E3 ubiquitin ligase which is cereblon (CRBN). They are represented by formula D1:

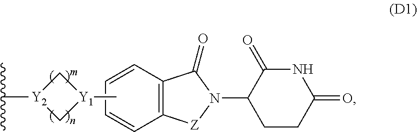

or a stereoisomer thereof
wherein,
Z is CH₂ or C═O;
Y₁ is N, CH, or COH;
Y₂ is N, CH, or cyclobutyl;
m is 1 or 2; and
n is 1 or 2.

In some embodiments, Z is CH₂.

In some embodiments, Z is C═O.

In some embodiments, Y₁ is N.

In some embodiments, Y₁ is CH.

In some embodiments, Y₁ is COH.

In some embodiments, Y₂ is N.

In some embodiments, Y₂ is CH.

In some embodiments, Y₂ is cyclobutyl.

In some embodiments, Y₁ is N and Y₂ is CH. In some embodiments, Y₁ is N and Y₂ is N. In some embodiments, Y₁ is N and Y₂ is cyclobutyl.

In some embodiments, Y₁ is CH and Y₂ is N. In some embodiments, Y₁ is COH and Y₂ is N.

In some embodiments, m is 1.

In some embodiments, m is 2.

In some embodiments, n is 1.

In some embodiments, n is 2.

In some embodiments, m is 1 and n is 1. In some embodiments, m is 2 and n is 1. In some embodiments, m is 1 and n is 2. In some embodiments, m is 2 and n is 2.

In some embodiments, formula D1 is of formula D1a to D1p.

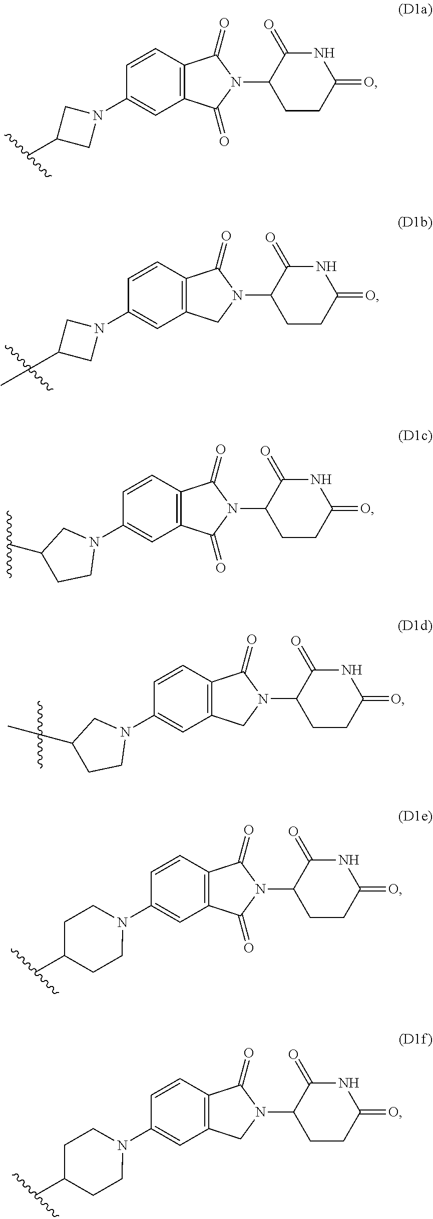 [SMILES: *C1CN(c2ccc3c(c2)C(=O)N(C2CCC(=O)NC2=O)C3=O)C1 ; *C1CN(c2ccc3c(c2)CN(C2CCC(=O)NC2=O)C3=O)C1 ; *C1CCN(c2ccc3c(c2)C(=O)N(C2CCC(=O)NC2=O)C3=O)C1 ; *C1CCN(c2ccc3c(c2)CN(C2CCC(=O)NC2=O)C3=O)C1 ; *C1CCN(c2ccc3c(c2)C(=O)N(C2CCC(=O)NC2=O)C3=O)CC1 ; *C1CCN(c2ccc3c(c2)CN(C2CCC(=O)NC2=O)C3=O)CC1]

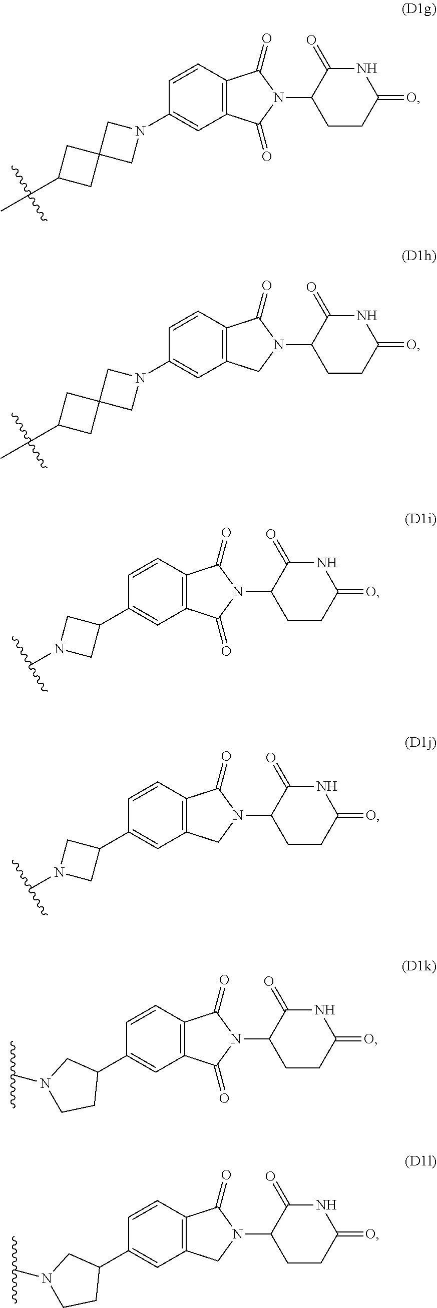 [SMILES: *C1CC2(C1)CN(c1ccc3c(c1)C(=O)N(C1CCC(=O)NC1=O)C3=O)C2 ; *C1CC2(C1)CN(c1ccc3c(c1)CN(C1CCC(=O)NC1=O)C3=O)C2 ; *N1CC(c2ccc3c(c2)C(=O)N(C2CCC(=O)NC2=O)C3=O)C1 ; *N1CC(c2ccc3c(c2)CN(C2CCC(=O)NC2=O)C3=O)C1 ; *N1CCC(c2ccc3c(c2)C(=O)N(C2CCC(=O)NC2=O)C3=O)C1 ; *N1CCC(c2ccc3c(c2)CN(C2CCC(=O)NC2=O)C3=O)C1]

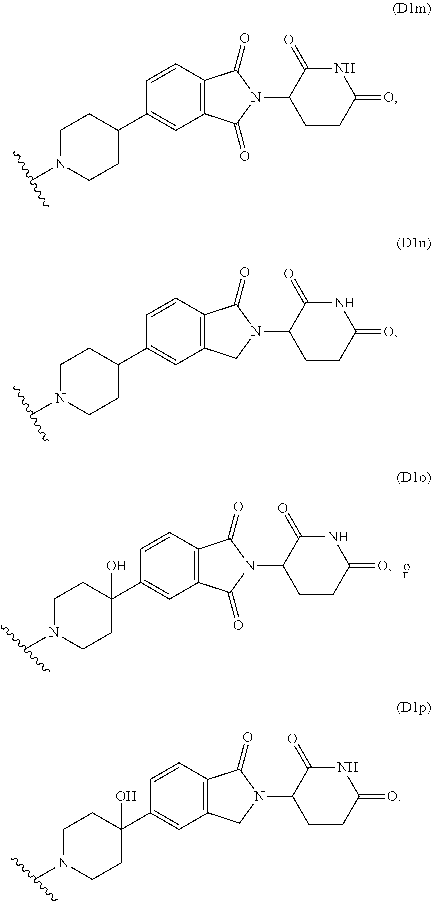 [SMILES: *N1CCC(c2ccc3c(c2)C(=O)N(C2CCC(=O)NC2=O)C3=O)CC1 ; *N1CCC(c2ccc3c(c2)CN(C2CCC(=O)NC2=O)C3=O)CC1 ; *N1CCC(O)(c2ccc3c(c2)C(=O)N(C2CCC(=O)NC2=O)C3=O)CC1 ; *N1CCC(O)(c2ccc3c(c2)CN(C2CCC(=O)NC2=O)C3=O)CC1]

Therefore, in some embodiments, the bifunctional compounds of this invention are represented by any structures generated by the combination of structures TL1 to TL3, L0 to L10, and the structures of the degron described herein, D1, or a pharmaceutically acceptable salt or stereoisomer thereof.

In some embodiments, the bifunctional compounds of the present invention are represented by any one of the following structures:

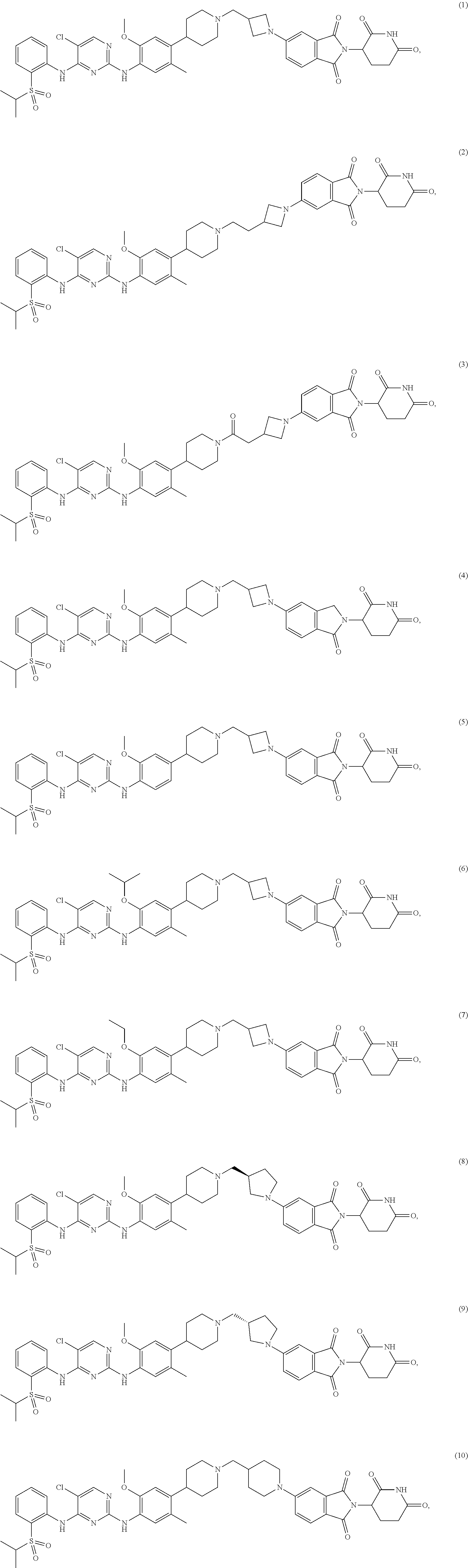

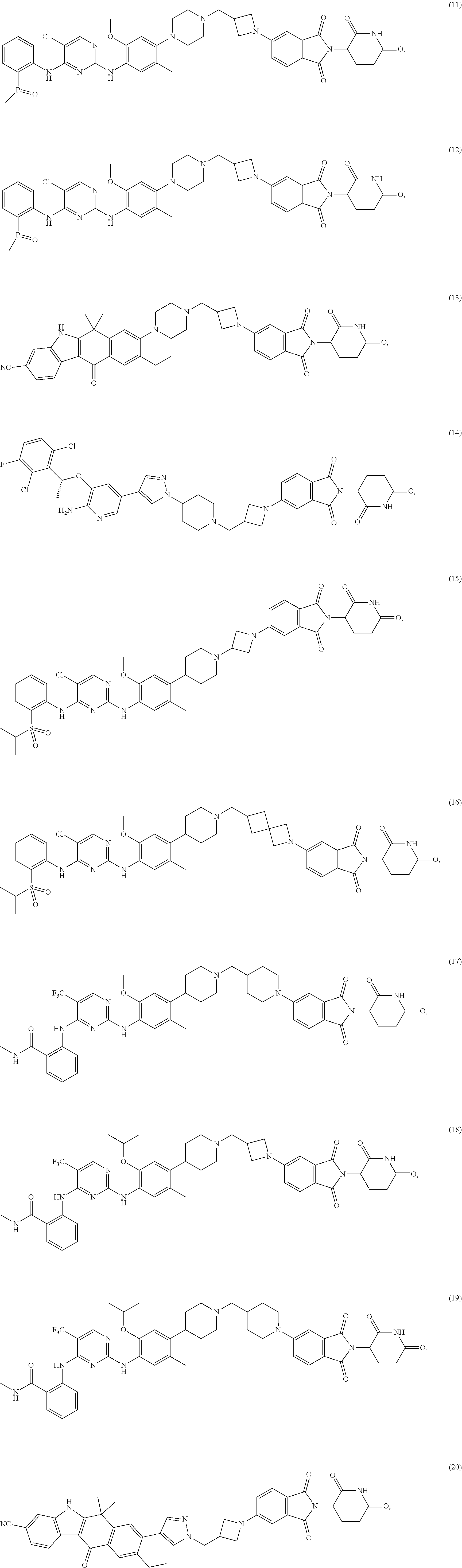

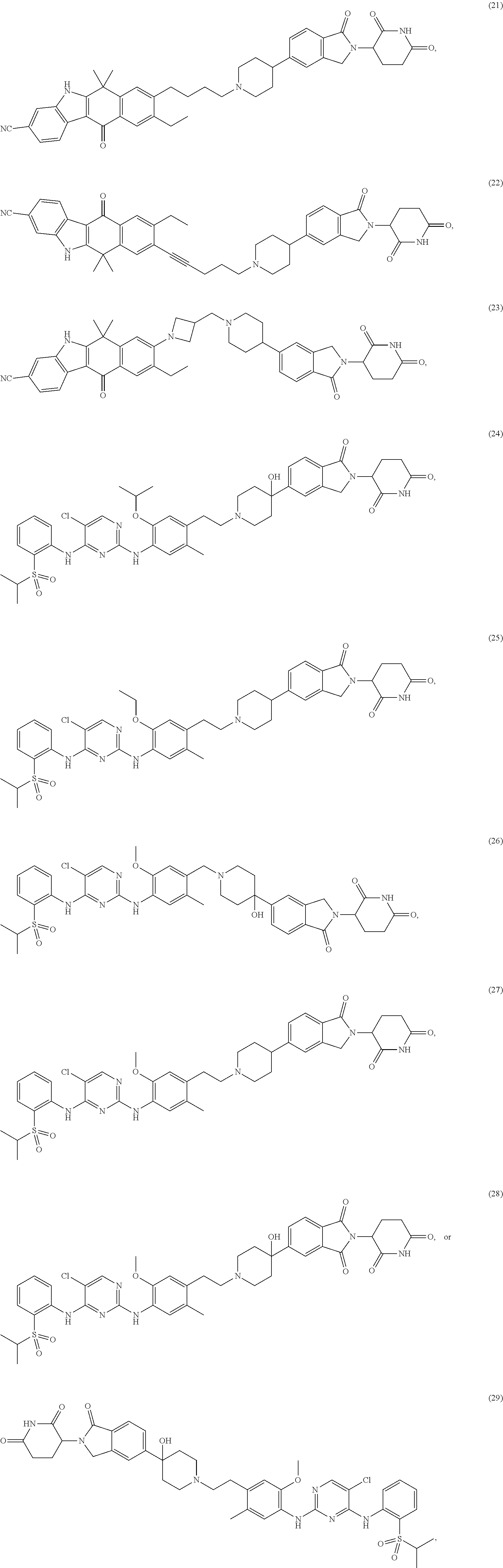

or a pharmaceutically acceptable salt or stereoisomer thereof.

Bifunctional compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of a bifunctional compound of the present invention with a suitable acid or a base. Examples of pharmaceutically acceptable salts of the bifunctional compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

Bifunctional compounds of the invention may have at least one chiral center and therefore may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; therefore, for these compounds, administration of the compound in its (R—) form is considered equivalent to administration of the bifunctional compound in its (S—) form. Accordingly, bifunctional compounds of the present invention may be made and used in the form of individual isomers and substantially free of other isomers, or in the form of a mixture of various isomers, e.g., racemic mixtures of stereoisomers.

The bifunctional compounds of the invention embrace isotopic derivatives that have at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and therefore may be advantageous in some circumstances.

In addition to isotopic derivatives, the term “bifunctional compounds of formula (I)” embraces N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds.

### Methods of Synthesis

In another aspect, the present invention is directed to a method for making a bifunctional compound of formula (I), or a pharmaceutically acceptable salt or stereoisomer thereof. Broadly, the inventive compounds or pharmaceutically-acceptable salts or stereoisomers thereof may be prepared by any process known to be applicable to the preparation of chemically related compounds. The bifunctional compounds of the present invention will be better understood in connection with the synthetic schemes that described in various working examples and which illustrate non-limiting methods by which they may be prepared.

### Pharmaceutical Compositions

Another aspect of the present invention is directed to a pharmaceutical composition that includes a therapeutically effective amount of a bifunctional compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, bifunctional compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal).

In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the bifunctional compound may be administered relatively quickly such as in the case of a single-dose treatment and/or an acute condition.

In some embodiments, the bifunctional compounds are formulated for oral or intravenous administration (e.g., systemic intravenous injection).

Accordingly, bifunctional compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Bifunctional compounds may also be formulated for rapid, intermediate or extended release.

Solid dosage forms for oral administration include capsules, tablets, pills, powders, and granules. In such solid dosage forms, the bifunctional compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the bifunctional compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bifunctional compounds of formula (I) may be administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the bifunctional compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The bifunctional compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of formula (I) may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating bifunctional compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the bifunctional compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the bifunctional compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bifunctional compound of formula (I) or a pharmaceutically acceptable salt or a stereoisomer thereof that is effective in producing the desired therapeutic response in a particular patient suffering from a disease or disorder mediated by aberrant ALK activity. The term “therapeutically effective amount” therefore includes the amount of the bifunctional compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer) cells, or reduces the amounts of ALK in diseased cells.

The total daily dosage of the bifunctional compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bifunctional compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, 2001).

Bifunctional compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, or in yet other embodiments from about 10 to about 30 mg per day. In some embodiments, the total daily dosage may range from 400 mg to 600 mg. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

In some embodiments, a bifunctional compound is administered in a dose between 100 mg per day and 250 mg per day. In other embodiments the bifunctional compound is administered in a dose between 200 mg per day and 400 mg per day, e.g., 250-350 mg per day.

### Methods of Use

In some aspects, the present invention is directed to treating diseases or disorders, cancerous and non-cancerous alike, characterized or mediated by aberrant (e.g., elevated levels of ALK or otherwise functionally abnormal e.g., deregulated ALK levels) ALK activity relative to a non-pathological state, which entails administering a therapeutically effective amount of a bifunctional compound of formula (I) or a pharmaceutically acceptable salt or stereoisomer thereof, to a subject in need thereof. A “disease” is generally regarded as a state of health of a subject wherein the subject cannot maintain homeostasis, and wherein if the disease is not ameliorated then the subject's health continues to deteriorate. In contrast, a “disorder” (or “condition”) in a subject is a state of health in which the subject is able to maintain homeostasis, but in which the subject's state of health is less favorable than it would be in the absence of the disorder. Left untreated, a disorder may or may not cause a further decrease in the subject's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Therefore, subjects suffering from, and suspected of suffering from, a specific disease or disorder are not necessarily two distinct groups.

In some embodiments, the inventive bifunctional compounds may be useful in the treatment of cell proliferative diseases and disorders (e.g., cancer or benign neoplasms). As used herein, the term “cell proliferative disease or disorder” refers to the conditions characterized by aberrant cell growth, or both, including noncancerous conditions such as neoplasms, precancerous conditions, benign tumors, and cancer.

Exemplary types of non-cancerous (e.g., cell proliferative) diseases or disorders that may be amenable to treatment with bifunctional compounds of the present invention include inflammatory diseases and conditions, autoimmune diseases, neurodegenerative diseases, heart diseases, viral diseases, chronic and acute kidney diseases or injuries, metabolic diseases, allergic disorders, and genetic diseases.

Representative examples of specific non-cancerous diseases and disorders include rheumatoid arthritis, alopecia areata, lymphoproliferative conditions, autoimmune hematological disorders (e.g., hemolytic anemia, aplastic anemia, anhidrotic ectodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniasis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcoidosis, bone resorption diseases, such as osteoporosis, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, uveitis, polymyositis, proctitis, interstitial lung fibrosis, dermatomyositis, atherosclerosis, arteriosclerosis, amyotrophic lateral sclerosis, asociality, varicosis, vaginitis, depression, and Sudden Infant Death Syndrome.

In some embodiments, the bifunctional compounds may be useful in the treatment of non-cancerous neurodegenerative diseases and disorders. As used herein, the term “neurodegenerative diseases and disorders” refers to the conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the bifunctional compounds may be useful in the treatment of autoimmune diseases and disorders. As used herein, the term “autoimmune disease” refers to conditions where the immune system produces antibodies that attack normal body tissues. Representative examples of such diseases include Sjogren's syndrome, Hashimoto thyroiditis, rheumatoid arthritis, juvenile (type 1) diabetes, polymyositis, scleroderma, Addison disease, lupus (e.g., systemic lupus erythematosus), vitiligo, pernicious anemia, glomerulonephritis, pulmonary fibrosis, celiac disease, polymyalgia rheumatica, multiple sclerosis, ankylosing spondylitis, alopecia areata, vasculitis, and temporal arteritis.

In some embodiments, the methods are directed to treating subjects having cancer. In some embodiments, the cancer is an ALK-positive cancer. In some embodiments, the cancer is an ALK-negative cancer. Broadly, the bifunctional compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinemia, melanoma, intraocular (eye) melanoma, Merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

In some embodiments, the cancer is anaplastic large cell lymphoma (ALCL), inflammatory myofibroblastic tumor (IMT), breast cancer, colorectal cancer, esophageal squamous cell cancer (ESCC), large B-cell lymphoma (DLBCL), renal cell cancer (RCC), or non-small cell lung cancer (NSCLC).

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types), and histiocytic sarcoma (immune cancer).

In some embodiments, methods of the present invention entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may therefore include leukemia, multiple myeloma, and lymphoma (including T-cell lymphoma, Hodgkin's lymphoma, non-Hodgkin's lymphoma (NHL). Examples of NHL include diffuse large B-cell lymphoma (DLBCL), follicular lymphoma (FL), mantle cell lymphoma (MCL), cutaneous T-cell lymphoma (CTCL) (including mycosis fungoides and Sezary syndrome), peripheral T-cell lymphoma (PTCL) (including anaplastic large-cell lymphoma (ALCL), angioimmunoblastic T-cell lymphoma, hepatosplenic T-cell lymphoma, epithelial T-cell lymphoma, and gamma-delta T-cell lymphoma), germinal center B-cell-like diffuse large B-cell lymphoma, activated B-cell-like diffuse large B-cell lymphoma, Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, refractory NHL, relapsed NHL, childhood lymphomas, and small lymphocytic lymphoma. Examples of leukemia include childhood leukemia, hairy-cell leukemia, acute lymphocytic leukemia, acute myelocytic leukemia, acute myeloid leukemia (e.g., acute monocytic leukemia), chronic lymphocytic leukemia, chronic myelocytic leukemia, chronic myelogenous leukemia, mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchoalveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a compound of the present invention may be used to treat non-metastatic or metastatic lung cancer (e.g., NSCLC, ALK-positive NSCLC, NSCLC harboring ROS1 Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

The bifunctional compounds of formula (I) and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been unsuccessful, or partially successful but who have become intolerant to the particular treatment. Therapy may also be given as an adjuvant treatment, i.e., to prevent reoccurrence of cancer in patients with no currently detectable disease or after surgical removal of a tumor. Therefore, in some embodiments, the compound may be administered to a patient who has received prior therapy, such as chemotherapy, radioimmunotherapy, surgical therapy, immunotherapy, radiation therapy, targeted therapy or any combination thereof.

The methods of the present invention may entail administration of a bifunctional compound of formula (I) or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day “off” period. In other embodiments, the bifunctional compound may be dosed twice a day (BID) over the course of two and a half days (for a total of 5 doses) or once a day (QD) over the course of two days (for a total of 2 doses). In other embodiments, the bifunctional compound may be dosed once a day (QD) over the course of 5 days.

### Combination Therapy

The bifunctional compounds of formula (I) and their pharmaceutically acceptable salts and stereoisomers may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Therefore, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Therefore, the terms are not limited to the administration of the active agents at exactly the same time.

In some embodiments, the treatment regimen may include administration of a bifunctional compound of formula (I) in combination with one or more additional therapeutics known for use in treating a disease or condition (e.g., cancer). The dosage of the additional therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006. For example, anti-cancer agents that may be suitable for use in combination with the inventive bifunctional compounds are known in the art. See, e.g., U.S. Pat. No. 9,101,622 (Section 5.2 thereof) and U.S. Pat. No. 9,345,705 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bifunctional antibodies) and CAR-T therapy.

In some embodiments, a bifunctional compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

When the active components of the combination are not administered in the same pharmaceutical composition, it is understood that they can be administered in any order to a subject in need thereof. For example, a bifunctional compound of the present invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional therapeutic, to a subject in need thereof. In various aspects, the therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the (e.g., anticancer) therapeutics are administered within the same office visit. In another example, the combination anticancer therapeutics may be administered at 1 minute to 24 hours apart.

In some embodiments involving cancer treatment, a bifunctional compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, the bifunctional compound of the present invention may be used in combination with other anti-cancer agents, examples of which include Paclitaxel (e.g., ovarian cancer, breast cancer, lung cancer, Kaposi sarcoma, cervical cancer, and pancreatic cancer), Topotecan (e.g., ovarian cancer and lung cancer), Irinotecan (e.g., colon cancer, and small cell lung cancer), Etoposide (e.g., testicular cancer, lung cancer, lymphomas, and non-lymphocytic leukemia), Vincristine (e.g., leukemia), Leucovorin (e.g., colon cancer), Altretamine (e.g., ovarian cancer), Daunorubicin (e.g., acute myeloid leukemia (AML), acute lymphocytic leukemia (ALL), chronic myelogenous leukemia (CML), and Kaposi's sarcoma), Trastuzumab (e.g., breast cancer, stomach cancer, and esophageal cancer), Rituximab (e.g., non-Hodgkin's lymphoma), Cetuximab (e.g., colorectal cancer, metastatic non-small cell lung cancer and head and neck cancer), Pertuzumab (e.g., metastatic HER2-positive breast cancer), Alemtuzumab (e.g., chronic lymphocytic leukemia (CLL), cutaneous T-cell lymphoma (CTCL) and T-cell lymphoma), Panitumumab (e.g., colon and rectum cancer), Tamoxifen (e.g., breast cancer), Fulvestrant (e.g., breast cancer), Letrazole (e.g., breast cancer), Exemestane (e.g., breast cancer), Azacytidine (e.g., myelodysplastic syndromes), Mitomycin C (e.g., gastro-intestinal cancers, anal cancers, and breast cancers), Dactinomycin (e.g., Wilms tumor, rhabdomyosarcoma, Ewing's sarcoma, trophoblastic neoplasm, testicular cancer, and ovarian cancer), Erlotinib (e.g., non-small cell lung cancer and pancreatic cancer), Sorafenib (e.g., kidney cancer and liver cancer), Temsirolimus (e.g., kidney cancer), Bortezomib (e.g., multiple myeloma and mantle cell lymphoma), Pegaspargase (e.g., acute lymphoblastic leukemia), Cabometyx (e.g., hepatocellular carcinoma, medullary thyroid cancer, and renal cell carcinoma), Keytruda (e.g., cervical cancer, gastric cancer, hepatocellular carcinoma, Hodgkin lymphoma, melanoma, Merkel cell carcinoma, non-small cell lung cancer, urothelial carcinoma, and squamous cell carcinoma of the head and neck), Nivolumab (e.g., colorectal cancer, hepatocellular carcinoma, melanoma, non-small cell lung cancer, renal cell carcinoma, small cell lung cancer, and urothelial carcinoma), Regorafenib (e.g., colorectal cancer, gastrointestinal stromal tumor, and hepatocellular carcinoma), and dexamethasone (e.g., acute multiple myeloma).

### Pharmaceutical Kits

The present bifunctional compounds and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a bifunctional compound of formula (I) or a pharmaceutical composition thereof. The kits or pharmaceutical systems of the invention may also include printed instructions for using the bifunctional compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: Synthesis of 5-{3-[(4-{4-[(5-chloro-4-{[2-(propane-2-sulfonyl)phenyl]amino}pyrimidin-2-yl)amino]-5-methoxy-2-methylphenyl}piperidin-1-yl)methyl]azetidin-1-yl}-2-(2,6-dioxopiperidin-3-yl)-2,3-dihydro-1H-isoindole-1,3-dione (1)

### 5-Chloro-N4-(2-(isopropylsulfonyl)phenyl)-N2-(2-methoxy-5-methyl-4-(piperidin-4-yl)phenyl)pyrimidine-2,4-diamine

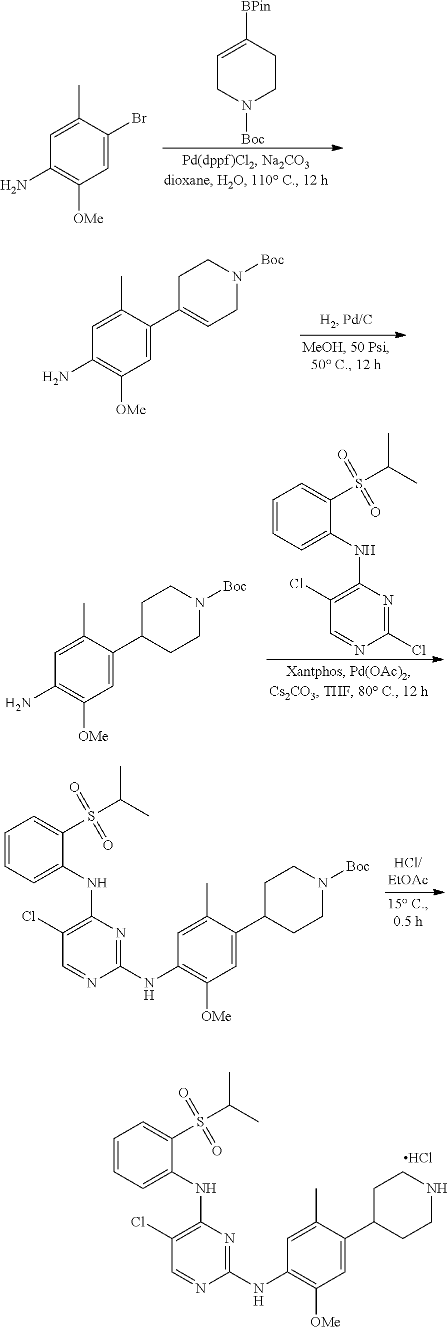

### tert-Butyl 4-(4-amino-5-methoxy-2-methylphenyl)-5,6-dihydropyridine-1(2H)-carboxylate

Na₂CO₃ (10.30 g, 97.19 mmol) and Pd(dppf)Cl₂ (1.69 g, 2.31 mmol) were added to a mixture of 4-bromo-2-methoxy-5-methyl-aniline (5.0 g, 23.14 mmol) and tert-butyl 4-(4,4,5,5-tetramethyl-1,3,2-dioxaborolan-2-yl)-3,6-dihydro-2H-pyridine-1-carboxylate (19.32 g, 62.48 mmol) in dioxane (100 mL) and H₂O (20 mL) and under nitrogen atmosphere the reaction mixture stirred at 110° C. 12 hours. Upon completion, the reaction mixture was quenched with water (150 mL) and extracted with EtOAc (200 mL×3). The combined organic phases were washed with brine (350 mL), dried with anhydrous Na₂SO₄, filtered and concentrated under pressure. The residue was purified by flash silica gel chromatography (ethyl acetate/petroleum ether, 20-50%) to give the title compound as a yellow solid (6.0 g, 78%). ¹H NMR (400 MHz, DMSO-d₆) δ 6.50 (s, 1H), 6.43 (s, 1H), 5.46 (br s, 1H), 4.57 (s, 2H), 3.92 (br s, 2H), 3.71 (s, 3H), 3.50 (t, J=5.5 Hz, 2H), 2.24 (brs, 2H), 2.06 (s, 3H), 1.43 (s, 9H).

### tert-Butyl 4-(4-amino-5-methoxy-2-methylphenyl)piperidine-1-carboxylate

Pd/C (3.0 g) was added to a solution of tert-butyl 4-(4-amino-5-methoxy-2-methyl-phenyl)-3,6-dihydro-2H-pyridine-1-carboxylate (6.0 g, 18.84 mmol) in MeOH (60 mL) under Argon. The suspension was degassed under vacuum and purged with H₂ three times. The reaction mixture stirred under H₂ (50 psi) at 50° C. for 12 hours. After completion of the reaction, the reaction mixture was filtered through a pad of Celite®, and the filtrate was concentrated under pressure to give a residue. The residue was purified by flash silica gel chromatography (ethyl acetate/petroleum ether, 15-40%) to give the title compound as a white solid (4.7 g, 75%). ¹H NMR (400M Hz, DMSO-d₆) δ 6.57 (s, 1H), 6.40 (s, 1H), 4.41 (s, 2H), 4.13-4.01 (m, 2H), 3.72 (s, 3H), 2.84-2.64 (m, 3H), 2.11 (s, 3H), 1.65-1.56 (m, 2H), 1.51-1.44 (m, 2H), 1.41 (s, 9H).

### tert-Butyl 4-(4-((5-chloro-4-((2-(isopropylsulfonyl)phenyl)amino)pyrimidin-2-yl)amino)-5-methoxy-2-methylphenyl)piperidine-1-carboxylate

Xantphos (1.70 g, 2.93 mmol), Cs₂CO₃ (14.34 g, 44.00 mmol) and Pd(OAc)₂ (329.31 mg, 1.47 mmol) were added to a mixture of tert-butyl 4-(4-amino-5-methoxy-2-methyl-phenyl)piperidine-1-carboxylate (4.7 g, 14.67 mmol) and 2,5-dichloro-N-(2-isopropylsulfonylphenyl)pyrimidin-4-amine (5.08 g, 14.67 mmol) in THE (100 mL) under N₂. The reaction mixture was heated to 80° C. and stirred for 12 hours. After completion of the reaction, the reaction mixture was quenched with water (200 mL) and extracted with EtOAc (150 mL×3). The combined organic phases were washed with brine (350 mL), dried with anhydrous Na₂SO₄, filtered and concentrated under pressure to give a residue. The residue was purified by reversed-phase HPLC (Biotage®, column: 800 g Agela; flow rate: 120 mL/min; mobile phase: H₂O; gradient B %: 50-80% 30 min; 80% 20 min) to give the title compound as a white solid (2.5 g, 27%). [M+H]⁺=630.2.

### 5-Chloro-N4-(2-(isopropylsulfonyl)phenyl)-N2-(2-methoxy-5-methyl-4-(piperidin-4-yl)phenyl)pyrimidine-2,4-diamine

A mixture of tert-butyl 4-[4-[[5-chloro-4-(2-isopropylsulfonylanilino)pyrimidin-2-yl]amino]-5-methoxy-2-methyl-phenyl]piperidine-1-carboxylate (2.5 g, 3.97 mmol) in HCl/EtOAc (4 M, 25.0 mL) stirred at 15° C. for 30 minutes. Following completion of the reaction, the reaction mixture was concentrated under reduced pressure to give the title compound as a white solid (2.5 g) which was used with further purification. ¹H NMR (400 MHz, DMSO-d₆) δ 10.09 (s, 1H), 9.42 (br s, 1H), 9.23 (br d, J=10.5 Hz, 1H), 9.10 (br d, J=10.5 Hz, 1H), 8.46 (s, 1H), 8.14 (br s, 1H), 7.90 (d, J=7.9 Hz, 1H), 7.71 (t, J=8.0 Hz, 1H), 7.52 (t, J=7.7 Hz, 1H), 7.27 (s, 1H), 6.80 (s, 1H), 3.77 (s, 3H), 3.48 (t, J=13.7, 6.7 Hz, 1H), 3.33 (br d, J=12.1 Hz, 2H), 3.07-2.96 (m, 3H), 2.06 (s, 3H), 1.99-1.90 (m, 2H), 1.77 (br d, J=12.7 Hz, 2H), 1.12 (d, J=6.8 Hz, 6H).

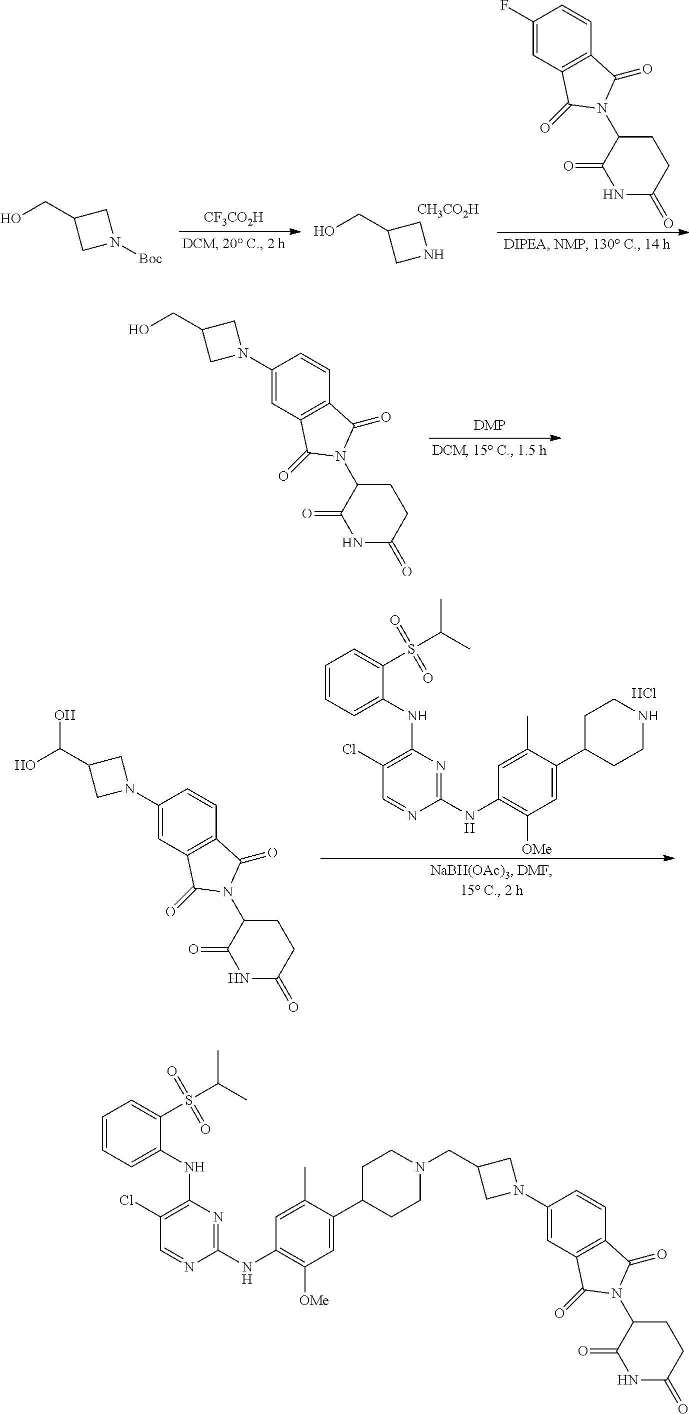

Azetidin-3-ylmethanol

TFA (8.0 mL, 108.05 mmol) was added to a mixture of tert-butyl 3-(hydroxymethyl)azetidine-1-carboxylate (5.0 g, 26.73 mmol) in DCM (20 mL) and the reaction mixture stirred at 20° C. for 2 hours. The mixture was concentrated under reduced pressure to give the title compound as a colorless oil (5.0 g) that was used without further purification.

### 2-(2,6-Dioxopiperidin-3-yl)-5-(3-(hydroxymethyl)azetidin-1-yl)isoindoline-1,3-dione

DIPEA (13.86 mL, 79.55 mmol) was added to a mixture of azetidin-3-ylmethanol (4 g, 9.94 mmol) and 2-(2,6-dioxo-3-piperidyl)-5-fluoro-isoindoline-1,3-dione (2.20 g, 7.95 mmol) in NMP (15 mL) and the reaction mixture stirred at 130° C. for 16 hours. After completion of the reaction, the reaction mixture was diluted with EtOAc (50 mL) and washed with H₂O (20 mL×3). The organic layer was dried over Na₂SO₄, filtered and concentrated under reduced pressure to give a residue. The residue was purified by column chromatography (SiO₂, petroleum ether/ethyl acetate=50-100%) to give the title compound as a yellow solid (3 g, 70%). [M+H]⁺=344.0.

### 1-[2-(2,6-Dioxo-3-piperidyl)-1,3-dioxo-isoindolin-5-yl]azetidine-3-carbaldehyde

DMP (4.06 mL, 13.11 mmol) was added to a mixture of 2-(2,6-dioxo-3-piperidyl)-5-[3-(hydroxymethyl)azetidin-1-yl]isoindoline-1,3-dione (3 g, 8.74 mmol) in DCM (30 mL) and the reaction mixture stirred at 15° C. for 1.5 hours. After completion of the reaction, the reaction mixture was quenched with sat. aq. NaHCO₃ (40 mL), sat. aq. Na₂S₂O₃ (40 mL), and extracted with EtOAc (60 mL×3). The combined organic phases were washed with brine (80 mL), dried with anhydrous Na₂SO₄, filtered and concentrated under pressure to give a residue. The residue was purified by flash silica gel chromatography (ethyl acetate/petroleum ether=20-100%) to give the title compound as a yellow solid (1.7 g, 50%). [M+H]⁺=360.0.

### 5-(3-((4-(4-((5-Chloro-4-((2-(isopropylsulfonyl)phenyl)amino)pyrimidin-2-yl)amino)-5-methoxy-2-methylphenyl)piperidin-1-yl)methyl)azetidin-1-yl)-2-(2,6-dioxopiperidin-3-yl)isoindoline-1,3-dione

Sodium triacetoxyborohydride (791.71 mg, 3.74 mmol) was added to a mixture of 1-[2-(2,6-dioxo-3-piperidyl)-1,3-dioxo-isoindolin-5-yl]azetidine-3-carbaldehyde (500 mg, 1.25 mmol) and 5-chloro-N4-(2-isopropylsulfonylphenyl)-N2-[2-methoxy-5-methyl-4-(4-piperidyl)phenyl]pyrimidine-2,4-diamine (705.44 mg, 1.25 mmol) in DMF (5 mL) and the reaction mixture stirred at 15° C. for 2 hours. After completion of the reaction, the reaction mixture was filtered and the filtrate was concentrated to give a residue. The residue was purified by prep-HPLC (column: Phenomenex Gemini® C18 250*50 mm*10 μm; mobile phase: [water(10 mM NH₄HCO₃)-ACN]; B %: 46%-80%, 20 min) to give the title compound as a yellow solid (102 mg). ¹H NMR (400 MHz, DMSO-d₆) δ 11.07 (s, 1H), 9.49 (s, 1H), 8.52 (br d, J=7.6 Hz, 1H), 8.27 (s, 1H), 8.23 (s, 1H), 7.82 (dd, J=1.3, 7.9 Hz, 1H), 7.66-7.56 (m, 2H), 7.42 (s, 1H), 7.33 (t, J=7.4 Hz, 1H), 6.87 (s, 1H), 6.79 (d, J=1.9 Hz, 1H), 6.65 (dd, J=8.4, 1.9 Hz, 1H), 5.12-4.99 (m, 1H), 4.16 (br t, J=8.1 Hz, 2H), 3.78 (s, 3H), 3.71 (br dd, J=8.0, 5.6 Hz, 2H), 3.44 (td, J=15.5, 6.7 Hz, 1H), 3.01 (br d, J=10.6 Hz, 3H), 2.89 (s, 1H), 2.71-2.57 (m, 4H), 2.16 (s, 3H), 2.14-1.92 (m, 4H), 1.78-1.65 (m, 4H), 1.16 (d, J=6.8 Hz, 6H). [M+H]⁺=855.2.

### Example 2: Synthesis of 3-(5-(3-((4-(4-((5-chloro-4-((2-(isopropylsulfonyl)phenyl)amino)pyrimidin-2-yl)amino)-5-methoxy-2-methylphenyl)piperidin-1-yl)methylazetidin-1-yl)-1-oxoisoindolin-2-yl)piperidine-2,6-dione (4)

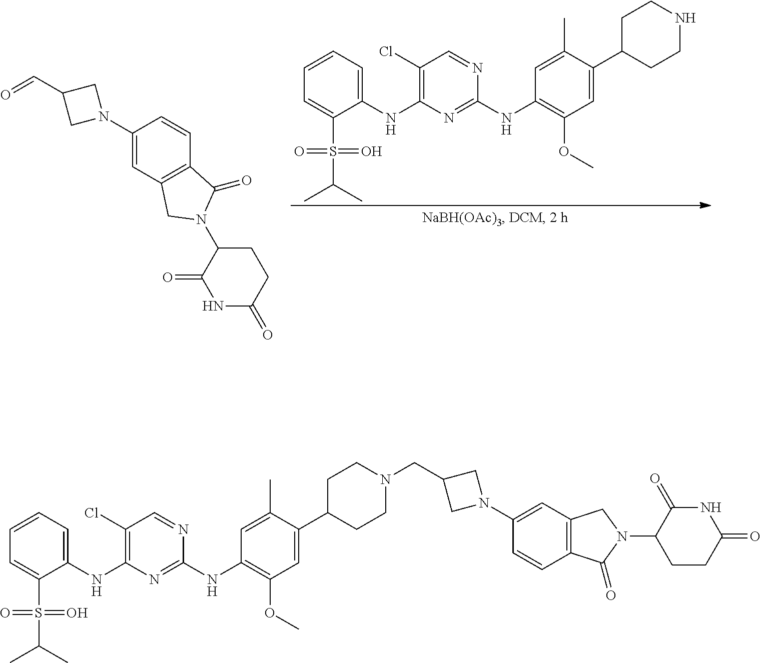 [SMILES: O=CC1CN(c2ccc3c(c2)CN(C2CCC(=O)NC2=O)C3=O)C1 ; COc1cc(C2CCNCC2)c(C)cc1Nc1ncc(Cl)c(Nc2ccccc2S(=O)(=O)C(C)C)n1 ; COc1cc(C2CCN(CC3CN(c4ccc5c(c4)CN(C4CCC(=O)NC4=O)C5=O)C3)CC2)c(C)cc1Nc1ncc(Cl)c(Nc2ccccc2S(=O)(=O)C(C)C)n1]

Compound 4 was prepared following a similar procedure as in Example 1. ¹H NMR (400 MHz, DMSO-d₆) δ 10.93 (s, 1H), 9.49 (s, 1H), 8.52 (d, J=8.5 Hz, 1H), 8.26 (s, 1H), 8.23 (s, 1H), 7.82 (dd, J=8.0, 1.6 Hz, 1H), 7.60 (t, J=8.0 Hz, 1H), 7.49 (d, J=8.3 Hz, 1H), 7.42 (s, 1H), 7.33 (t, J=7.7 Hz, 1H), 6.87 (s, 1H), 6.53-6.45 (m, 2H), 5.03 (dd, J=13.3, 5.1 Hz, 1H), 4.30 (d, J=16.9 Hz, 1H), 4.18 (d, J=16.8 Hz, 1H), 4.04 (t, J=7.8 Hz, 2H), 3.78 (s, 3H), 3.62-3.55 (m, 2H), 3.45 (p, J=6.8 Hz, 1H), 2.99 (d, J=10.5 Hz, 3H), 2.69-2.59 (m, 4H), 2.41-2.26 (m, 1H), 2.09 (t, J=10.4 Hz, 1H), 2.01-1.90 (m, 1H), 1.81-1.58 (m, 5H), 1.17 (s, 3H), 1.15 (s, 3H). [M+H]⁺=842.4.

### Example 3: Synthesis of 5-(3-((4-(4-((5-chloro-4-((2-(isopropylsulfonyl)phenyl)amino)pyrimidin-2-yl)amino)-3-methoxyphenyl)piperidin-1-yl)methyl)azetidin-1-yl)-2-(2,6-dioxopiperidin-3-yl)isoindoline-1,3-dione (5)

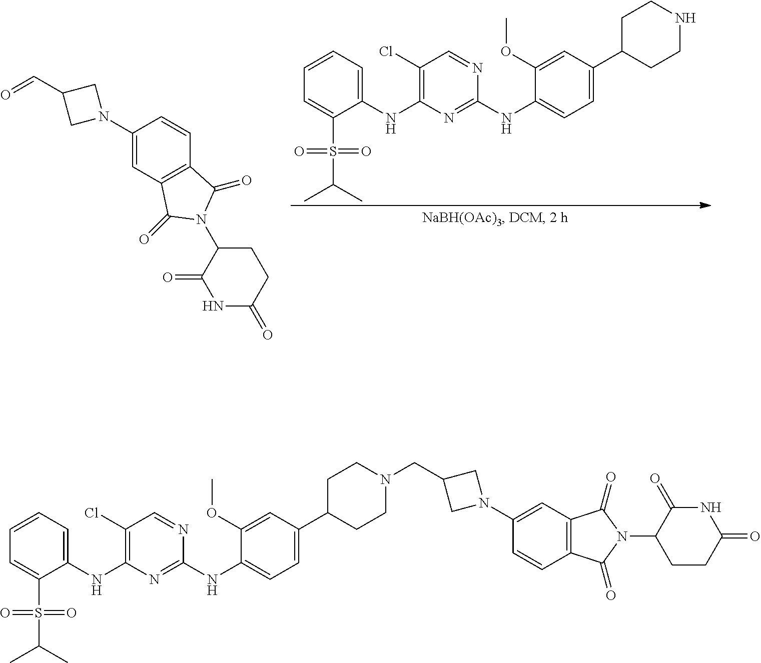 [SMILES: O=CC1CN(c2ccc3c(c2)C(=O)N(C2CCC(=O)NC2=O)C3=O)C1 ; COc1cc(C2CCNCC2)ccc1Nc1ncc(Cl)c(Nc2ccccc2S(=O)(=O)C(C)C)n1 ; COc1cc(C2CCN(CC3CN(c4ccc5c(c4)C(=O)N(C4CCC(=O)NC4=O)C5=O)C3)CC2)ccc1Nc1ncc(Cl)c(Nc2ccccc2S(=O)(=O)C(C)C)n1]

Compound 5 was prepared following a similar procedure as in Example 1. [M+H]⁺=842.4.

### Example 4: Synthesis of 8-[3-(2-{4-[2-(2,6-dioxopiperidin-3-yl)-1-oxo-2,3-dihydro-1H-isoindol-5-yl]piperidin-1-yl}ethyl)azetidin-1-yl]-9-ethyl-6,6-dimethyl-11-oxo-5H,6H,11H-benzo[b]carbazole-3-carbonitrile (6)

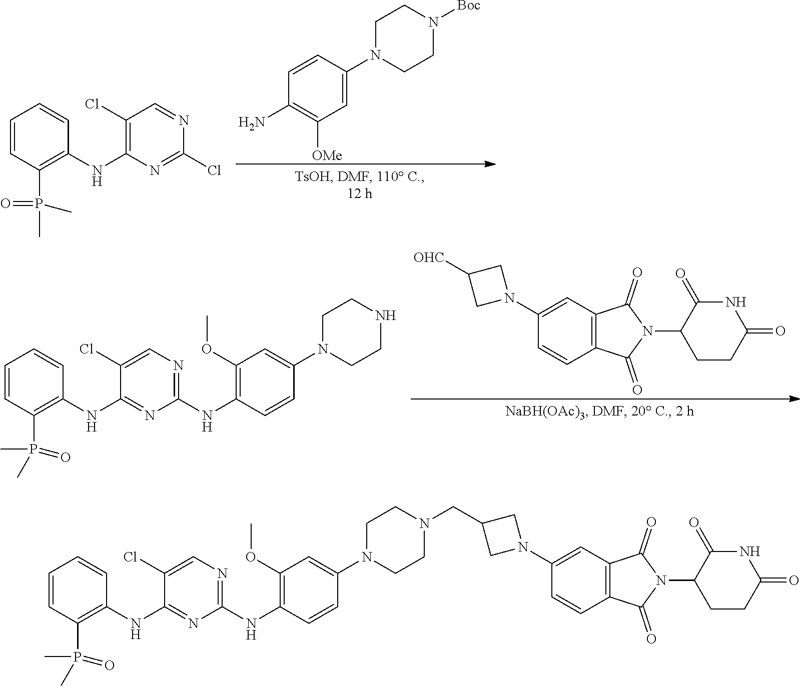 [SMILES: CP(C)(=O)c1ccccc1Nc1nc(Cl)ncc1Cl ; COc1cc(N2CCN(C(=O)OC(C)(C)C)CC2)ccc1N ; COc1cc(N2CCNCC2)ccc1Nc1ncc(Cl)c(Nc2ccccc2P(C)(C)=O)n1 ; O=CC1CN(c2ccc3c(c2)C(=O)N(C2CCC(=O)NC2=O)C3=O)C1 ; COc1cc(N2CCN(CC3CN(c4ccc5c(c4)C(=O)N(C4CCC(=O)NC4=O)C5=O)C3)CC2)ccc1Nc1ncc(Cl)c(Nc2ccccc2P(C)(C)=O)n1]

A mixture of 5-chloro-N2-[2-isopropoxy-5-methyl-4-(4-piperidyl)phenyl]-N4-(2-isopropylsulfonylphenyl)pyrimidine-2,4-diamine (200 mg, 336.36 μmol) and 1-[2-(2,6-dioxo-3-piperidyl)-1,3-dioxo-isoindolin-5-yl]azetidine-3-carbaldehyde (183.69 mg, 538.18 μmol) in MeOH (1.5 mL), DMF (1.5 mL) and HOAc (0.1 mL) stirred at 15° C. for 1 hour under N₂. NaBH(OAc)₃ (142.58 mg, 672.73 μmol) was then added. The reaction mixture stirred at 15° C. for 11 hours. The reaction mixture was concentrated under reduced pressure to give a residue. The residue was purified by prep-HPLC (column: Waters™ Xbridge BEH C18 100*25 mm*5 μm; mobile phase: [water (10 mM NH₄HCO₃)-ACN]; B %: 65%-85%, 10 min) to give the title compound as a yellow solid (94.5 mg, 30%). ¹H NMR (400 MHz, DMSO-d₆) δ 11.07 (br s, 1H), 9.46 (br s, 1H), 8.47 (br d, J=8.2 Hz, 1H), 8.25 (s, 1H), 8.05 (s, 1H), 7.83 (dd, J=8.0, 1.5 Hz, 1H), 7.66-7.60 (m, 2H), 7.51 (s, 1H), 7.36 (t, J=7.2 Hz, 1H), 6.85 (s, 1H), 6.79 (d, J=2.0 Hz, 1H), 6.65 (dd, J=8.5, 2.0 Hz, 1H), 5.05 (dd, J=12.8, 5.4 Hz, 1H), 4.59 (td, J=12.1, 6.0 Hz, 1H), 4.15 (t, J=8.2 Hz, 2H), 3.70 (dd, J=8.2, 5.6 Hz, 2H), 3.51-3.38 (m, 1H), 3.09-2.82 (m, 4H), 2.69-2.53 (m, 5H), 2.14-1.96 (m, 6H), 1.71-1.60 (m, 4H), 1.22 (d, J=6.1 Hz, 6H), 1.67 (d, J=6.8 Hz, 6H). [M+H⁺]=883.3.

### Example 5: Synthesis of 5-(3-((4-(4-((5-chloro-4-((2-(isopropylsulfonyl)phenyl)amino)pyrimidin-2-yl)amino)-5-ethoxy-2-methylphenyl)piperidin-1-yl)methyl)azetidin-1-yl)-2-(2,6-dioxopiperidin-3-yl)isoindoline-1,3-dione (7)

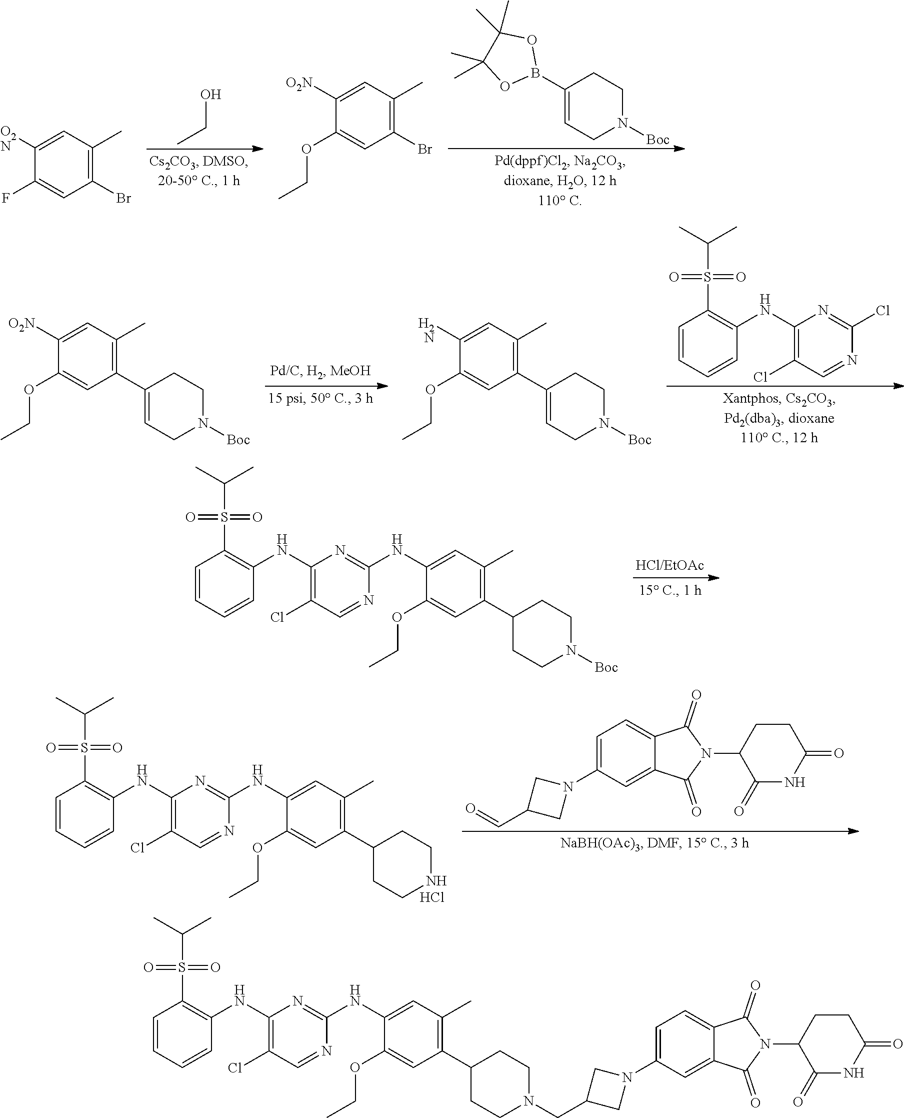

### 1-Bromo-5-ethoxy-2-methyl-4-nitrobenzene

Cs₂CO₃ (6.96 g, 21.37 mmol) and ethanol (0.65 mL, 11.22 mmol) were added sequentially to a solution of 1-bromo-5-fluoro-2-methyl-4-nitro-benzene (1.0 g, 4.27 mmol) in DMSO (10 mL) at 20° C. The reaction mixture was heated to 50° C. and stirred for 1 hour under N2. The reaction solution was poured into iced water (15 mL) and ethyl acetate (40 mL×3). The combined organic phases were dried with anhydrous Na₂SO₄. After filtering, the organic solvent was removed under pressure to give a residue. The residue was purified by flash silica gel chromatography (ethyl acetate/petroleum ether, 0-5%) to give the title compound as a yellow solid (900 mg, 80%). ¹H NMR (400 MHz, DMSO-d₆) δ 8.09 (s, 1H), 7.77 (s, 1H), 4.39 (q, J=6.8 Hz, 2H), 2.68 (br s, 3H), 1.49 (t, J=6.8 Hz, 3H).

### tert-Butyl 4-(5-ethoxy-2-methyl-4-nitrophenyl)-5,6-dihydropyridine-1(2H)-carboxylate

Na₂CO₃ (1.37 g, 12.92 mmol) and Pd(dppf)Cl₂ (450.13 mg, 615.19 μmol) were added to a mixture of 1-bromo-5-ethoxy-2-methyl-4-nitro-benzene (800 mg, 3.08 mmol) and tert-butyl 4-(4,4,5,5-tetramethyl-1,3,2-dioxaborolan-2-yl)-3,6-dihydro-2H-pyridine-1-carboxylate (2.57 g, 8.31 mmol) in dioxane (16 mL) and H₂O (3.2 mL) under N₂. The reaction mixture was heated to 110° C. and stirred for 12 hours. The reaction mixture was quenched with water (20 mL) and then extracted with EtOAc (40 mL×3). The combined organic phases were washed with brine (80 mL), dried with anhydrous Na₂SO₄, filtered and concentrated under pressure to give a residue that was purified by flash silica gel chromatography (ethyl acetate/petroleum ether, 15-25%) to give the title compound as a yellow solid (1.0 g, 85%). [M+H⁺]=307.2.

### tert-Butyl 4-(4-amino-5-ethoxy-2-methylphenyl)piperidine-1-carboxylate

Pd/C (0.5 g, 137.96 μmol) was added to a solution of tert-butyl 4-(5-ethoxy-2-methyl-4-nitro-phenyl)-3,6-dihydro-2H-pyridine-1-carboxylate (1.0 g, 2.76 mmol) in MeOH (25 mL) under Ar. The suspension was degassed under vacuum and purged with H₂ several times. The mixture stirred under H₂ (15 psi) at 50° C. for 3 hours. The reaction mixture was filtered through a pad of Celite® and the filtrate was concentrated under reduced pressure to give a residue. The crude product was purified by reversed-phase MPLC (Biotage®, column: 120 g Agela C18; flow rate: 50 mL/min; mobile phase; H₂O; gradient B %: 50-80% 10 min; 80% 10 min) to give the title compound as a colorless oil (500 mg, 52%). ¹H NMR (400 MHz, DMSO-d₆) δ 6.56 (s, 1H), 6.41 (s, 1H), 4.39 (s, 2H), 4.15-4.00 (m, 2H), 3.94 (q, J=6.8 Hz, 2H), 2.90-2.62 (m, 3H), 2.11 (s, 3H), 1.59 (br d, J=12.8 Hz, 2H), 1.41 (s, 9H), 1.29 (t, J=6.8 Hz, 3H). [M+H⁺]=279.1.

### tert-Butyl 4-(4-((5-chloro-4-((2-(isopropylsulfonyl)phenyl)amino)pyrimidin-2-yl)amino)-5-ethoxy-2-methylphenyl)piperidine-1-carboxylate

A mixture of tert-butyl 4-(4-amino-5-ethoxy-2-methyl-phenyl)piperidine-1-carboxylate (500 mg, 1.49 mmol), 2,5-dichloro-N-(2-isopropylsulfonylphenyl)pyrimidin-4-amine (517.61 mg, 1.49 mmol), Pd₂(dba)₃ (34.22 mg, 37.37 μmol), Xantphos (43.25 mg, 74.75 μmol) and Cs₂CO₃ (730.64 mg, 2.24 mmol) in dioxane (5 mL) was degassed under vacuum and purged with N₂ for 5 minutes. The reaction mixture stirred at 110° C. under N₂ for 12 hours. The reaction mixture was quenched with water (10 mL) and the aq. phase was extracted with ethyl acetate (15 mL×3). The combined organic phases were washed with brine (25 mL), dried with anhydrous Na₂SO₄, filtered and concentrated in vacuum. The residue was purified by flash silica gel chromatography (ethyl acetate/petroleum ether, 20-30%) to give the title compound as a pale yellow solid (250 mg, 25%). ¹H NMR (400 MHz, DMSO-d₆) δ 9.48 (s, 1H), 8.48 (br d, J=8.0 Hz, 1H), 8.24 (s, 1H), 8.17 (s, 1H), 7.83 (dd, J=8.0, 1.6 Hz, 1H), 7.60 (br t, J=7.2 Hz, 1H), 7.47 (s, 1H), 7.38-7.31 (m, 1H), 6.82 (s, 1H), 4.20-3.98 (m, 5H), 3.50-3.39 (m, 1H), 2.94-2.76 (m, 3H), 2.15 (s, 3H), 1.71-1.63 (m, 2H), 1.60-1.48 (m, 2H), 1.43 (s, 9H), 1.25 (t, J=6.8 Hz, 3H), 1.18-1.14 (m, 6H). [M+H⁺]=644.3.

### 5-Chloro-N₂-(2-ethoxy-5-methyl-4-(piperidin-4-yl)phenyl)-N4-(2-(isopropylsulfonyl)phenyl)pyrimidine-2,4-diamine hydrochloride

A mixture of tert-butyl 4-[4-[[5-chloro-4-(2-isopropylsulfonylanilino)pyrimidin-2-yl]amino]-5-ethoxy-2-methyl-phenyl]piperidine-1-carboxylate (50 mg, 77.61 μmol) in HCl/EtOAc (4 M, 4.00 mL) stirred at 15° C. for 1 hour. The reaction mixture was concentrated under reduced pressure to give the title compound as a white solid (50 mg) and used without further purification. [M+H⁺]=544.2.

### 5-(3-((4-(4-((5-Chloro-4-((2-(isopropylsulfonyl)phenyl)amino)pyrimidin-2-yl)amino)-5-ethoxy-2-methylphenyl)piperidin-1-yl)methyl)azetidin-1-yl)-2-(2,6-dioxopiperidin-3-yl)isoindoline-1,3-dione

Sodium triacetoxyborohydride (54.76 mg, 258.37 μmol) was added to a solution of 5-chloro-N2-[2-ethoxy-5-methyl-4-(4-piperidyl)phenyl]-N4-(2-isopropylsulfonylphenyl)pyrimidine-2,4-diamine (50 mg, 86.12 μmol) and 1-[2-(2,6-dioxo-3-piperidyl)-1,3-dioxo-isoindolin-5-yl]azetidine-3-carbaldehyde (41.99 mg, 86.12 μmol) in DMF (0.5 mL) and the reaction mixture stirred at 15° C. for 3 hours. The reaction mixture (combined with another batch at 100 mg scale) was filtered and the filtrate was purified by prep-HPLC (column: Phenomenex Gemini®-NX 80*40 mm*3 μm; mobile phase: [water(10 mM NH₄HCO₃)-ACN]; B %: 40%-70%, 8 min) to give the title compound as a yellow solid (37.3 mg). ¹H NMR (400 MHz, DMSO-d₆) δ 11.07 (s, 1H), 9.48 (s, 1H), 8.49 (br d, J=8.4 Hz, 1H), 8.24 (s, 1H), 8.16 (s, 1H), 7.83 (dd, J=8.0, 1.6 Hz, 1H), 7.66-7.56 (m, 2H), 7.46 (s, 1H), 7.36 (t, J=7.6 Hz, 1H), 6.84 (s, 1H), 6.79 (d, J=2.0 Hz, 1H), 6.65 (dd, J=8.4, 2.0 Hz, 1H), 5.05 (dd, J=12.8, 5.2 Hz, 1H), 4.15 (br t, J=8.4 Hz, 2H), 4.05 (q, J=6.8 Hz, 2H), 3.71 (br dd, J=8.0, 5.6 Hz, 2H), 3.50-3.40 (m, 1H), 3.06-2.82 (m, 4H), 2.68-2.59 (m, 4H), 2.15-2.05 (m, 5H), 2.04-1.97 (m, 1H), 1.73-1.63 (m, 4H), 1.27 (t, J=6.8 Hz, 3H), 1.16 (d, J=6.8 Hz, 6H). [M+H⁺]=869.2.

### Example 6: Synthesis of 5-[(3R)-3-[(4-{4-[(5-chloro-4-{[2-(propane-2-sulfonyl)phenyl]amino}pyrimidin-2-yl)amino]-5-methoxy-2-methylphenyl}piperidin-1-yl)methyl]pyrrolidin-1-yl]-2-(2,6-dioxopiperidin-3-yl)-2,3-dihydro-1H-isoindole-1,3-dione (8)

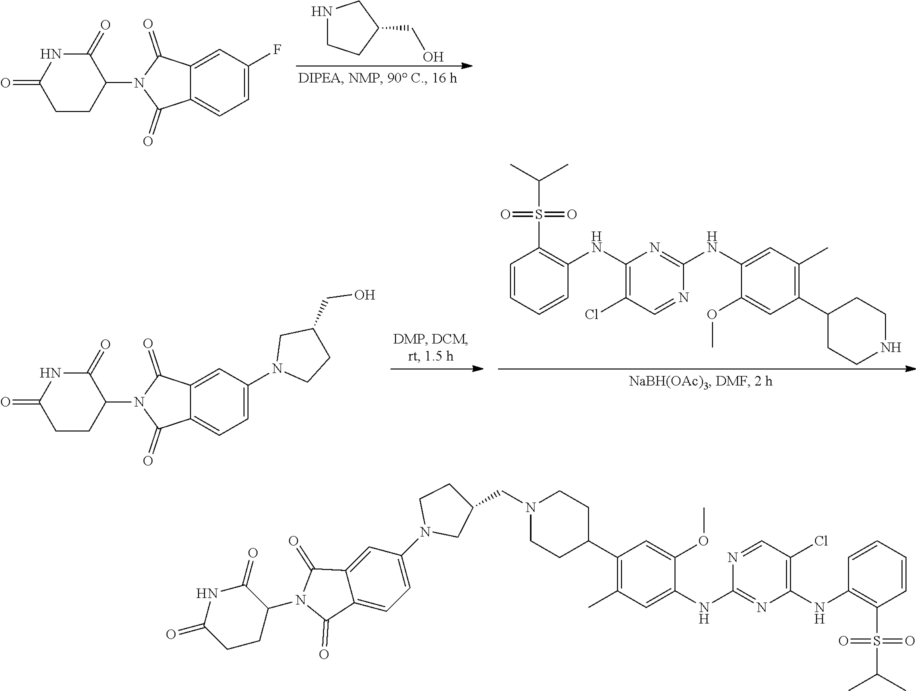 [SMILES: O=C1CCC(N2C(=O)c3ccc(F)cc3C2=O)C(=O)N1 ; OC[C@@H]1CCNC1 ; O=C1CCC(N2C(=O)c3ccc(N4CC[C@@H](CO)C4)cc3C2=O)C(=O)N1 ; COc1cc(C2CCNCC2)c(C)cc1Nc1ncc(Cl)c(Nc2ccccc2S(=O)(=O)C(C)C)n1 ; COc1cc(C2CCN(C[C@H]3CCN(c4ccc5c(c4)C(=O)N(C4CCC(=O)NC4=O)C5=O)C3)CC2)c(C)cc1Nc1ncc(Cl)c(Nc2ccccc2S(=O)(=O)C(C)C)n1]

### 2-(2,6-Dioxopiperidin-3-yl)-5-((R)-3-(hydroxymethyl)pyrrolidin-1-yl)isoindoline-1,3-dione

A mixture of 2-(2,6-dioxopiperidin-3-yl)-5-fluoroisoindoline-1,3-dione (400 mg, 1.45 mmol), NMP (5 mL), (R)-pyrrolidin-3-ylmethanol (146.47 mg, 1.45 mmol) and DIPEA (1.01 mL, 5.79 mmol) stirred at 90° C. for 16 hours. After completion of the reaction, the reaction mixture diluted with DCM (10 mL) and washed with H₂O (5 mL×3), dried over Na₂SO₄, filtered and concentrated under reduced pressure to give a residue. The residue was purified by prep-TLC (SiO₂, 100% ethyl acetate) to give the title compound as a yellow solid (400 mg, 77%). ¹H NMR (400 MHz, CDCl₃) δ 8.71 (s, 1H), 7.60 (d, J=8.3 Hz, 1H), 6.91 (s, 1H), 6.64 (br d, J=8.3 Hz, 1H), 4.92 (br dd, J=11.6, 5.0 Hz, 1H), 3.73-3.68 (m, 1H), 3.66-3.59 (m, 1H), 3.55-3.49 (m, 1H), 3.48-3.43 (m, 1H), 3.41-3.34 (m, 3H), 3.27-3.20 (m, 1H), 2.88-2.76 (m, 6H), 2.75-2.70 (m, 1H), 2.60 (td, J=14.0, 6.9 Hz, 1H), 2.36 (t, J=8.1 Hz, 3H), 2.19-2.09 (m, 2H), 2.05-1.95 (m, 3H), 1.93-1.82 (m, 1H).

### (3R)-1-(2-(2,6-dioxopiperidin-3-yl)-1,3-dioxoisoindolin-5-yl)pyrrolidine-3-carbaldehyde

DMP (356.06 mg, 839.49 μmol) was added to a mixture of 2-(2,6-dioxo-3-piperidyl)-5-[(3S)-3-(hydroxymethyl)pyrrolidin-1-yl]isoindoline-1,3-dione (200.00 mg, 559.66 μmol) in DCM (1 mL) and the mixture stirred at 20° C. for 1.5 hours. After completion of the reaction, the reaction mixture was quenched with NaHCO₃ (5 mL) and diluted with DCM (10 mL) and then extracted with DCM (10 mL×3). The combined organic layers were washed with Na₂CO₃ (10 mL×3), dried over Na₂SO₄, filtered and concentrated under reduced pressure to give a residue. The residue was purified by prep-TLC (SiO₂, 100% ethyl acetate) to give the title compound as a white solid (100 mg, 44%). ¹H NMR (400 MHz, CDCl₃) δ 9.78 (s, 1H), 8.10 (br s, 1H), 7.69 (d, J=8.4 Hz, 1H), 6.99 (d, J=1.9 Hz, 1H), 6.73 (dd, J=8.4, 2.0 Hz, 1H), 4.95 (dd, J=12.2, 5.2 Hz, 1H), 3.83 (dd, J=10.3, 4.6 Hz, 1H), 3.59 (dd, J=10.2, 7.8 Hz, 1H), 3.55-3.41 (m, 2H), 3.34-3.24 (m, 1H), 2.92-2.70 (m, 3H), 2.48-2.32 (m, 2H), 2.18-2.09 (m, 1H).

### 5-(3-((4-(4-((5-Chloro-4-((2-(isopropylsulfonyl)phenyl)amino)pyrimidin-2-yl)amino)-5-methoxy-2-methylphenyl)piperidin-1-yl)methyl)pyrrolidin-1-yl)-2-(2,6-dioxopiperidin-3-yl)isoindoline-1,3-dione

5-Chloro-N4-(2-(isopropylsulfonyl)phenyl)-N2-(2-methoxy-5-methyl-4-(piperidin-4-yl)phenyl)pyrimidine-2,4-diamine (131.27 mg, 247.65 μmol) and NaBH(OAc)₃ (209.95 mg, 990.59 μmol) were added to a solution of (3R)-1-(2-(2,6-dioxopiperidin-3-yl)-1,3-dioxoisoindolin-5-yl) pyrrolidine-3-carbaldehyde (100 mg, 247.65 μmol) in DMF (5 mL) and the reaction mixture stirred at 20° C. for 2 hours. After completion of the reaction, the reaction mixture was filtered and the filtrate was purified by prep-HPLC (column: Phenomenex Gemini®-NX 80*40 mm*3 μm; mobile phase: [water (10 mM NH₄HCO₃)-ACN]; B %: 50%-80%, 8 min) to give the title compound as a yellow solid (44 mg, 20%). ¹H NMR (400 MHz, DMSO-d₆) δ 11.06 (s, 1H), 9.49 (s, 1H), 8.52 (br d, J=7.5 Hz, 1H), 8.24 (br d, J=10.5 Hz, 2H), 7.82 (br d, J=6.7 Hz, 1H), 7.68-7.57 (m, 2H), 7.43 (s, 1H), 7.38-7.30 (m, 1H), 6.94-6.85 (m, 2H), 6.84-6.79 (m, 1H), 5.05 (dd, J=12.8, 5.4 Hz, 1H), 3.82-3.76 (m, 3H), 3.63-3.39 (m, 4H), 3.22-3.02 (m, 3H), 2.97-2.81 (m, 1H), 2.74-2.57 (m, 4H), 2.40 (br d, J=7.4 Hz, 2H), 2.20-2.01 (m, 7H), 1.85-1.65 (m, 5H), 1.16 (d, J=6.8 Hz, 6H). [M+H⁺]=869.2.

### Example 7: Synthesis of 5-[(3S)-3-[(4-{4-[(5-chloro-4-{[2-(propane-2-sulfonyl)phenyl]amino}pyrimidin-2-yl)amino]-5-methoxy-2-methylphenyl}piperidin-1-yl)methyl]pyrrolidin-1-yl-2-(2,6-dioxopiperidin-3-yl)-2,3-dihydro-1H-isoindole-1,3-dione (9)

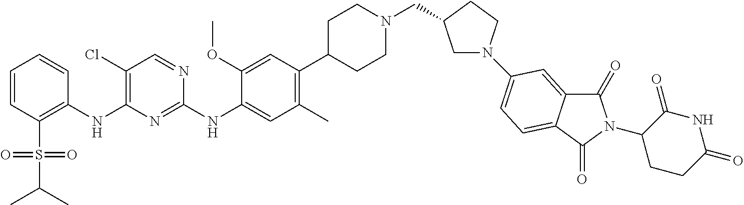 [SMILES: COc1cc(C2CCN(C[C@@H]3CCN(c4ccc5c(c4)C(=O)N(C4CCC(=O)NC4=O)C5=O)C3)CC2)c(C)cc1Nc1ncc(Cl)c(Nc2ccccc2S(=O)(=O)C(C)C)n1]

Compound 9 was prepared following a similar procedure as in Example 6. ¹H NMR (400 MHz, DMSO-d₆) δ 11.06 (s, 1H), 9.49 (s, 1H), 8.52 (br d, J=8.3 Hz, 1H), 8.29-8.21 (m, 2H), 7.87-7.78 (m, 1H), 7.68-7.57 (m, 2H), 7.43 (s, 1H), 7.36-7.30 (m, 1H), 6.93-6.87 (m, 2H), 6.82 (dd, J=8.5, 1.9 Hz, 1H), 5.05 (dd, J=12.8, 5.4 Hz, 1H), 3.79 (s, 3H), 3.64-3.56 (m, 1H), 3.51 (br s, 1H), 3.47-3.39 (m, 2H), 3.23-2.99 (m, 4H), 2.94-2.82 (m, 1H), 2.72-2.59 (m, 4H), 2.40 (br d, J=7.3 Hz, 2H), 2.19-2.01 (m, 7H), 1.81-1.65 (m, 5H), 1.16 (d, J=6.8 Hz, 6H). [M+H⁺]=869.3.

### Example 8: Synthesis of 5-{4-[(4-{4-[(5-chloro-4-{[2-(propane-2-sulfonyl)phenyl]amino]pyrimidin-2-yl)amino]-5-methoxy-2-methylphenyl}piperidin-1-yl)methyl}piperidin-1-yl}-2-(2,6-dioxopiperidin-3-yl)-2,3-dihydro-1H-isoindole-1,3-dione (10)

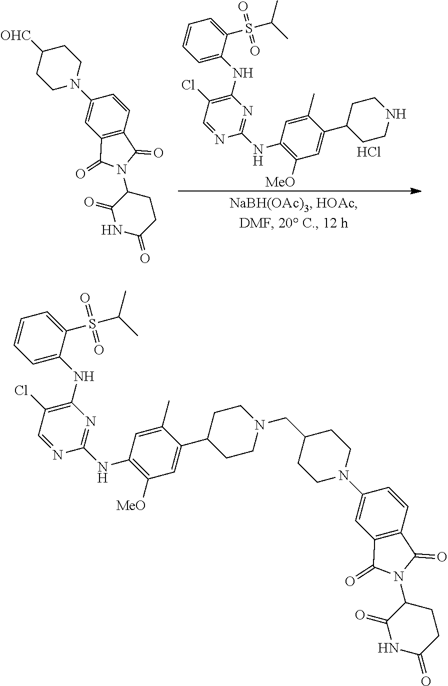 [SMILES: O=CC1CCN(c2ccc3c(c2)C(=O)N(C2CCC(=O)NC2=O)C3=O)CC1 ; COc1cc(C2CCNCC2)c(C)cc1Nc1ncc(Cl)c(Nc2ccccc2S(=O)(=O)C(C)C)n1 ; Cl ; COc1cc(C2CCN(CC3CCN(c4ccc5c(c4)C(=O)N(C4CCC(=O)NC4=O)C5=O)CC3)CC2)c(C)cc1Nc1ncc(Cl)c(Nc2ccccc2S(=O)(=O)C(C)C)n1]

NaBH(OAc)₃ (29.93 mg, 141.21 μmol) was added to a solution of 5-chloro-N4-(2-isopropylsulfonylphenyl)-N2-[2-methoxy-5-methyl-4-(4-piperidyl)phenyl]pyrimidine-2,4-diamine (40 mg, 70.60 μmol) and 1-[2-(2,6-dioxo-3-piperidyl)-1,3-dioxo-isoindolin-5-yl]piperidine-4-carbaldehyde (26.08 mg, 70.60 μmol) in DMF (5 mL) and HOAc (0.05 mL) and the reaction mixture stirred at 20° C. for 12 hours. The reaction mixture was filtered and the filtrate was purified by prep-HPLC (column: Welch Xtimate® C18 100*25 mm*3 μm; mobile phase: [water(0.05% HCl)-ACN]; B %: 35%-65%, 8 min) to give the title compound as a yellow solid (43.5 mg, 85%). ¹H NMR (400 MHz, DMSO-d₆) δ 11.08 (s, 1H), 10.41 (br s, 1H), 9.95 (br s, 1H), 9.17 (br s, 1H), 8.40 (s, 1H), 8.23 (br s, 1H), 7.89 (dd, J=7.9, 1.3 Hz, 1H), 7.74-7.64 (m, 2H), 7.47 (t, J=7.6 Hz, 1H), 7.36 (d, J=1.7 Hz, 1H), 7.34-7.25 (m, 2H), 6.93 (s, 1H), 5.07 (dd, J=12.8, 5.4 Hz, 1H), 4.10 (br d, J=13.0 Hz, 2H), 3.77 (s, 3H), 3.60 (br d, J=11.0 Hz, 2H), 3.48 (quind, J=13.5, 6.7 Hz, 1H), 3.38-3.23 (m, 1H), 3.16-2.95 (m, 6H), 2.94-2.80 (m, 1H), 2.63-2.52 (m, 2H), 2.44-2.30 (m, 2H), 2.22 (br s, 1H), 2.11 (s, 3H), 2.00 (br d, J=12.6 Hz, 3H), 1.82 (br d, J=13.1 Hz, 2H), 1.39-1.24 (m, 2H), 1.14 (d, J=6.8 Hz, 6H). [M+H+]=883.4.

### Example 9: Synthesis of 5-{3-[(4-{4-[(5-chloro-4-{[2-(dimethylphosphoryl)phenyl]amino}pyrimidin-2-yl)aminol-3-methoxyphenyl}piperazin-1-yl)methyl]azetidin-1-yl}-2-(2,6-dioxopiperidin-3-yl)-2,3-dihydro-1H-isoindole-1,3-dione (11)

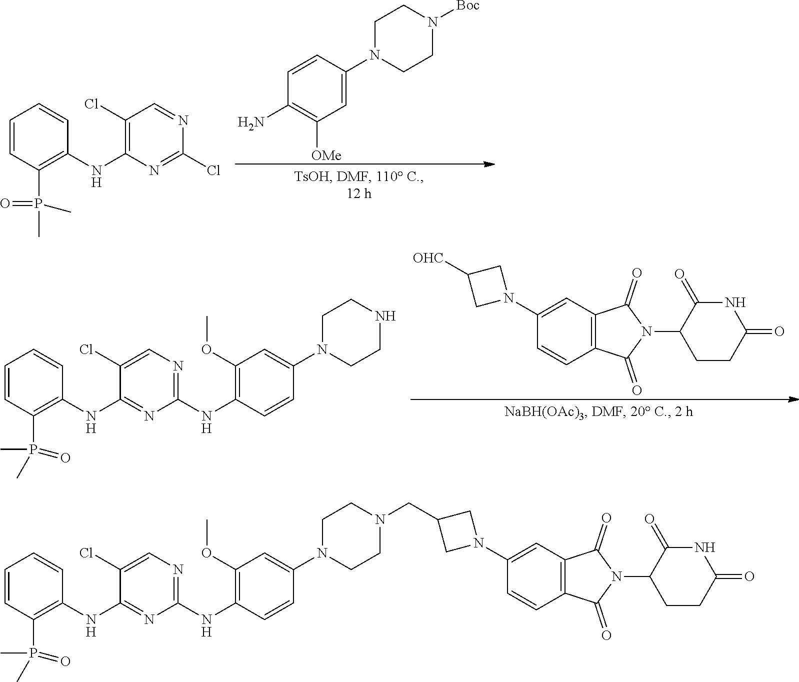 [SMILES: CP(C)(=O)c1ccccc1Nc1nc(Cl)ncc1Cl ; COc1cc(N2CCN(C(=O)OC(C)(C)C)CC2)ccc1N ; COc1cc(N2CCNCC2)ccc1Nc1ncc(Cl)c(Nc2ccccc2P(C)(C)=O)n1 ; O=CC1CN(c2ccc3c(c2)C(=O)N(C2CCC(=O)NC2=O)C3=O)C1 ; COc1cc(N2CCN(CC3CN(c4ccc5c(c4)C(=O)N(C4CCC(=O)NC4=O)C5=O)C3)CC2)ccc1Nc1ncc(Cl)c(Nc2ccccc2P(C)(C)=O)n1]

### (2-((5-Chloro-2-((2-methoxy-4-(piperazin-1-yl)phenyl)amino)pyrimidin-4-yl)amino)phenyl)dimethylphosphine oxide

TsOH (571.96 mg, 3.32 mmol) was added to a mixture of 2,5-dichloro-N-(2-dimethylphosphorylphenyl)pyrimidin-4-amine (700 mg, 2.21 mmol) and tert-butyl 4-(4-amino-3-methoxy-phenyl)piperazine-1-carboxylate (816.79 mg, 2.66 mmol) in DMF (12 mL) under N₂ and the reaction mixture was stirred at 110° C. for 12 hours. The reaction mixture was cooled to 20° C. and concentrated under reduced pressure. The residue was poured into saturated NaHCO₃ (20 mL) and stirred for 10 minutes. The aqueous phase was extracted with ethyl acetate (40 mL×3). The combined organic phases were washed with brine (50 mL), dried with anhydrous Na₂SO₄, filtered and concentrated under reduced pressure. The residue was purified by silica gel column chromatography (DCM/MeOH=10/1 to 0/1) to give the title compound as a yellow solid (250 mg, 19%). ¹H NMR (400 MHz, DMSO-d₆) δ 11.17 (brs, 1H), 8.46 (brs, 1H), 8.06 (brs, 2H), 7.56-7.51 (m, 1H), 7.41-7.31 (m, 2H), 7.08 (brs, 1H), 6.64-6.61 (m, 1H), 6.45 (brs, 1H), 3.76 (s, 3H), 3.17 (s, 2H), 3.01 (s, 4H), 2.84 (s, 1H), 1.77-1.74 (d, J=13.6 Hz, 6H), 1.27-1.17 (m, 1H), 0.93-0.85 (m, 1H). [M+H⁺]=487.2.

### 5-(3-((4-(4-((5-Chloro-4-((2-(dimethylphosphoryl)phenyl)amino)pyrimidin-2-yl)amino)-3-methoxyphenyl)piperazin-1-yl)methyl)azetidin-1-yl)-2-(2,6-dioxopiperidin-3-yl)isoindoline-1,3-dione

NaBH(OAc)₃ (87.05 mg, 410.73 μmol) was added to a mixture of 5-chloro-N₄-(2-dimethylphosphorylphenyl)-N2-(2-methoxy-4-piperazin-1-yl-phenyl)pyrimidine-2,4-diamine (100 mg, 205.37 μmol) and 1-[2-(2,6-dioxo-3-piperidyl)-1,3-dioxo-isoindolin-5-yl]azetidine-3-carbaldehyde (140.19 mg, 205.37 μmol) in DMF (4.5 mL) and the mixture was stirred at 20° C. for 2 hours. The reaction mixture was filtered and the filtrate was purified by prep-HPLC (column: Waters™ Xbridge BEH C18 100*30 mm*10 μm; mobile phase: [water (10 mM NH₄HCO₃)-ACN]; B %: 40%-70%, 8 min) to give the title compound as a yellow solid (39.1 mg, 22%). ¹H NMR (400 MHz, DMSO-d₆) δ 11.18 (s, 1H), 11.07 (s, 1H), 8.48 (brs, 1H), 8.07 (br d, J=3.6 Hz, 2H), 7.64 (d, J=8.2 Hz, 1H), 7.58-7.48 (m, 1H), 7.40 (br d, J=8.6 Hz, 1H), 7.35 (br t, J=7.6 Hz, 1H), 7.10 (br t, J=7.2 Hz, 1H), 6.79 (d, J=1.9 Hz, 1H), 6.68-6.62 (m, 2H), 6.48 (dd, J=8.6, 2.2 Hz, 1H), 5.05 (dd, J=12.9, 5.2 Hz, 1H), 4.16 (br t, J=8.0 Hz, 2H), 3.77 (s, 3H), 3.73 (br dd, J=8.1, 5.6 Hz, 2H), 3.15 (br s, 4H), 3.11-3.01 (m, 1H), 2.93-2.82 (m, 1H), 2.68 (br d, J=7.7 Hz, 2H), 2.62-2.54 (m, 6H), 2.05-1.94 (m, 1H), 1.78 (s, 3H), 1.75 (s, 3H). [M+H⁺]=812.2.

### Example 10: Synthesis of 8-(4-((1-(2-(2,6-dioxopiperidin-3-yl)-1,3-dioxoisoindolin-5-yl)azetidin-3-yl)methyl)piperazin-1-yl)-9-ethyl-6,6-dimethyl-11-oxo-6,11-dihydro-5H-benzo[b]carbazole-3-carbonitrile (13)

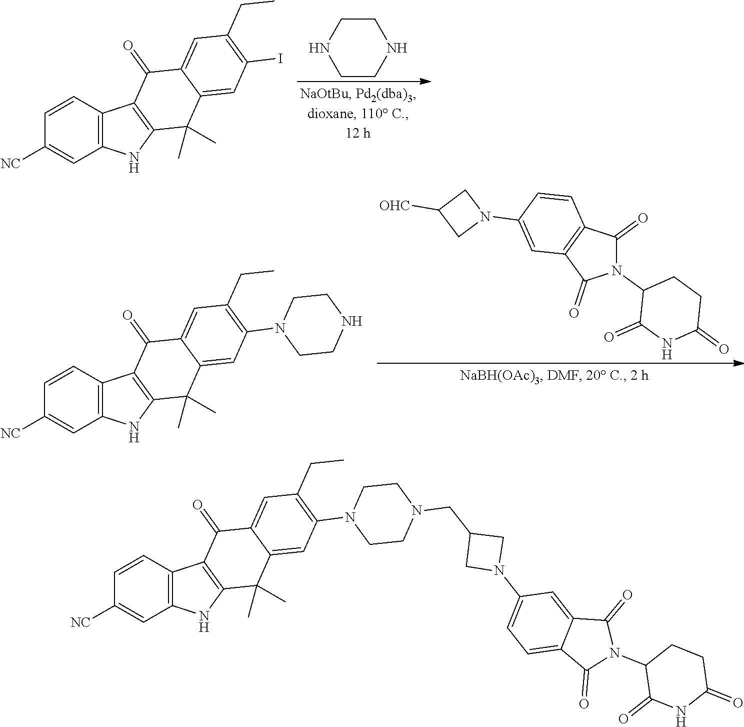

### 9-Ethyl-6,6-dimethyl-11-oxo-8-(piperazin-1-yl)-6,11-dihydro-5H-benzo[b]carbazole-3-carbonitrile

NaO^{t}Bu (1.57 g, 16.35 mmol), Pd₂(dba)₃ (1.03 g, 1.12 mmol) and tris-o-tolylphosphane (622.17 mg, 2.04 mmol) were added to a mixture of 9-ethyl-8-iodo-6,6-dimethyl-11-oxo-5H-benzo[b]carbazole-3-carbonitrile (900 mg, 2.04 mmol) and piperazine (281.72 mg, 3.27 mmol) in dioxane (50 mL) under N₂ and the reaction mixture was stirred at 110° C. for 12 hours. After completion of the reaction, the reaction mixture was quenched with water (100 mL) and then extracted with EtOAc (100 mL×3). The combined organic layers were washed with brine (250 mL), dried over anhydrous Na₂SO₄, filtered and concentrated under reduced pressure to give a residue. The residue was purified by reversed-phase HPLC (Biotage®, column: 330 g Agela C18; flow rate: 70 mL/min; mobile phase: H₂O; gradient B %: 40-70% 25 min; 70% 10 min) to give the title compound as a yellow solid (230 mg, 22%). [M+H⁺]=399.2.

### 8-(4-((1-(2-(2,6-Dioxopiperidin-3-yl)-1,3-dioxoisoindolin-5-yl)azetidin-3-yl)methyl)piperazin-1-yl)-9-ethyl-6,6-dimethyl-11-oxo-6,11-dihydro-5H-benzo[b]carbazole-3-carbonitrile

1-[2-(2,6-Dioxo-3-piperidyl)-1,3-dioxo-isoindolin-5-yl]azetidine-3-carbaldehyde (8.56 mg, 25.09 μmol) and sodium triacetoxyborohydride (10.64 mg, 50.18 μmol) were added to a solution of 9-ethyl-6,6-dimethyl-11-oxo-8-piperazin-1-yl-5H-benzo[b]carbazole-3-carbonitrile (20 mg, 25.09 μmol) in DMF (0.5 mL) and the reaction mixture was stirred at 20° C. for 2 hours. After completion of the reaction, the reaction mixture was filtered and the filtrate was concentrated under reduced pressure to give a residue. The residue was purified by prep-HPLC (column: Waters™ Xbridge BEH C18 100*30 mm*10 μm; mobile phase: [water(10 mM NH₄HCO₃)-ACN]; B %: 40%-70%, 8 min) to give the title compound as a yellow solid (6.0 mg, 31%). ¹H NMR (400 MHz, DMSO-d₆) δ 12.71 (br s, 1H), 11.07 (s, 1H), 8.32 (d, J=8.2 Hz, 1H), 8.05 (s, 1H), 7.99 (s, 1H), 7.67-7.57 (m, 2H), 7.38 (s, 1H), 6.79 (d, J=1.8 Hz, 1H), 6.65 (dd, J=8.4, 1.9 Hz, 1H), 5.06 (dd, J=12.8, 5.4 Hz, 1H), 4.15 (br t, J=8.1 Hz, 2H), 3.72 (br dd, J=7.9, 5.5 Hz, 2H), 3.10-2.87 (m, 6H), 2.76-2.68 (m, 4H), 2.60 (br s, 4H), 2.07 (s, 2H), 2.05-1.98 (m, 1H), 1.76 (s, 6H), 1.27 (t, J=7.5 Hz, 3H). [M+H+]=724.2.

### Example 11: Synthesis of 5-(3-{[4-(4-{6-amino-5-[(1R)-1-(2,6-dichloro-3-fluorophenyl)ethoxypyridin-3-yl}-1H-pyrazol-1-yl)piperidin-1-yl]methyl}azetidin-1-yl)-2-(2,6-dioxopiperidin-3-yl)-2,3-dihydro-1H-isoindole-1,3-dione (14)

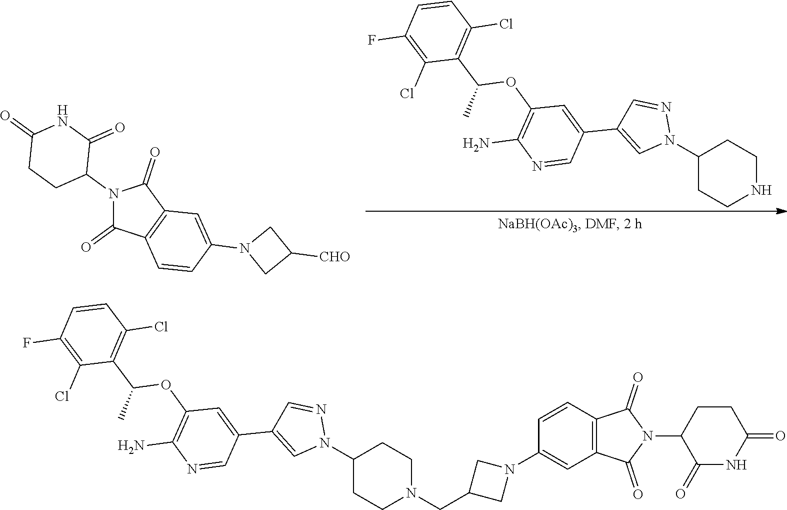 [SMILES: O=CC1CN(c2ccc3c(c2)C(=O)N(C2CCC(=O)NC2=O)C3=O)C1 ; C[C@@H](Oc1cc(-c2cnn(C3CCNCC3)c2)cnc1N)c1c(Cl)ccc(F)c1Cl ; C[C@@H](Oc1cc(-c2cnn(C3CCN(CC4CN(c5ccc6c(c5)C(=O)N(C5CCC(=O)NC5=O)C6=O)C4)CC3)c2)cnc1N)c1c(Cl)ccc(F)c1Cl]

Compound 14 was prepared following a similar procedure as in Example 10. ¹H NMR (400 MHz, DMSO-d₆) δ 11.06 (s, 1H), 7.97 (s, 1H), 7.76 (d, J=1.7 Hz, 1H), 7.63 (d, J=8.3 Hz, 1H), 7.57 (dd, J=9.0, 4.9 Hz, 1H), 7.53 (s, 1H), 7.44 (t, J=8.7 Hz, 1H), 6.97-6.86 (m, 1H), 6.78 (d, J=2.1 Hz, 1H), 6.64 (dd, J=8.3, 2.1 Hz, 1H), 6.08 (q, J=6.6 Hz, 1H), 5.63 (s, 2H), 5.05 (dd, J=12.9, 5.4 Hz, 1H), 4.12 (dt, J=11.1, 6.5 Hz, 3H), 3.70 (dd, J=8.4, 5.3 Hz, 2H), 3.11-2.80 (m, 4H), 2.74-2.54 (m, 3H), 2.24-2.07 (m, 2H), 2.07-1.85 (m, 5H), 1.80 (d, J=6.6 Hz, 3H). [M+H⁺]=724.2.

### Example 12: Synthesis of 5-[3-(4-{4-[(5-chloro-4-{[2-(propane-2-sulfonyl)phenyl]amino}pyrimidin-2-yl)amino]-5-methoxy-2-methylphenyl}piperidin-1-yl)azetidin-1-yl]-2-(2,6-dioxopiperidin-3-yl)-2,3-dihydro-1H-isoindole-1,3-dione (15)

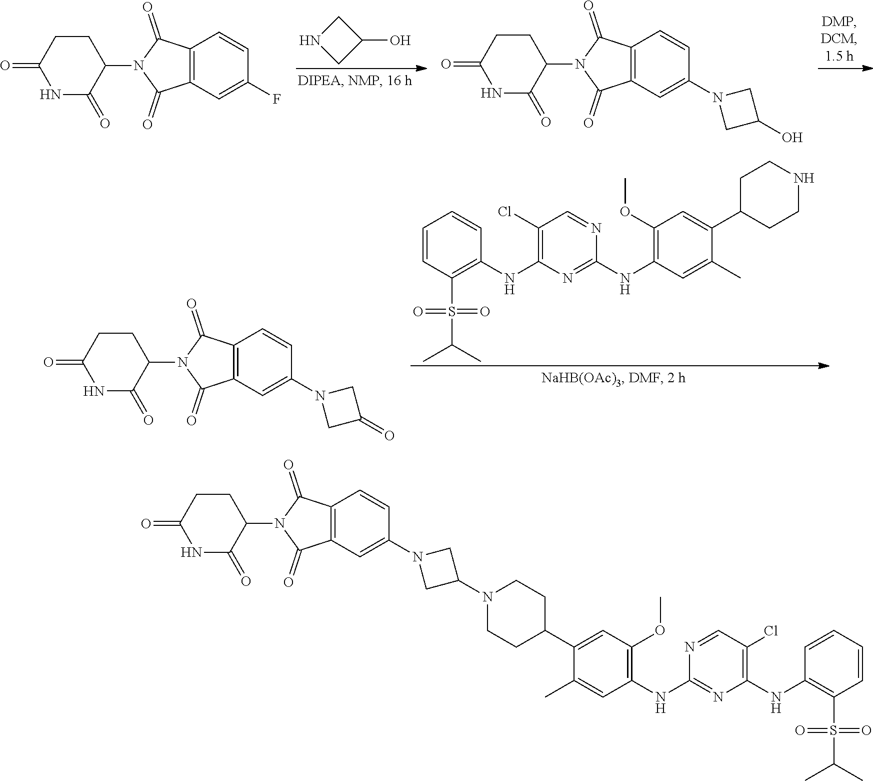

Compound 15 was prepared following a similar procedure as in Example 1. ¹H NMR (400 MHz, DMSO-d₆) δ 11.08 (s, 1H), 9.49 (s, 1H), 8.52 (d, J=8.1 Hz, 1H), 8.29 (s, 1H), 8.23 (s, 1H), 7.81 (dd, J=8.0, 1.7 Hz, 1H), 7.66 (d, J=8.4 Hz, 1H), 7.59 (t, J=7.9 Hz, 1H), 7.41 (s, 1H), 7.37-7.29 (m, 1H), 6.88 (s, 1H), 6.82 (d, J=2.1 Hz, 1H), 6.68 (dd, J=8.5, 2.2 Hz, 1H), 5.06 (dd, J=12.9, 5.4 Hz, 1H), 4.15 (t, J=7.8 Hz, 2H), 3.91-3.82 (m, 2H), 3.76 (s, 3H), 3.44 (p, J=6.9 Hz, 1H), 3.31 (s, 1H), 2.99 (d, J=10.4 Hz, 2H), 2.94-2.78 (m, 1H), 2.76-2.66 (m, 2H), 2.59 (s, 2H), 2.33 (p, J=1.8 Hz, 1H), 2.16 (s, 3H), 2.01 (s, 3H), 1.73 (s, 4H), 1.17 (s, 3H), 1.15 (s, 3H). [M+H⁺]=842.4.

### Example 13: Synthesis of 5-(6-((4-(4-((5-chloro-4-((2-(isopropylsulfonyl)phenyl)amino)pyrimidin-2-yl)amino)-5-methoxy-2-methylphenyl)piperidin-1-yl)methyl)-2-azaspiro[3.3]heptan-2-yl)-2-(2,6-dioxopiperidin-3-yl)isoindoline-1,3-dione (16)

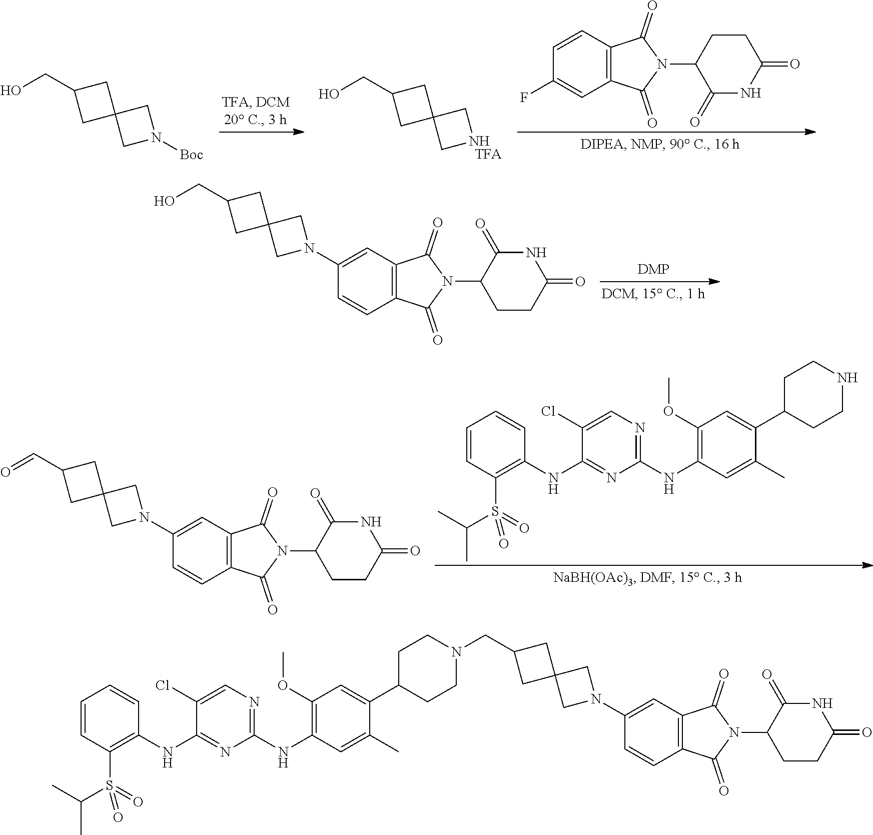

### 2-Azaspiro[3.3]heptan-6-ylmethanol

TFA (4.0 mL, 54.02 mmol) was added to a solution of tert-butyl 6-(hydroxymethyl)-2-azaspiro[3.3]heptane-2-carboxylate (400 mg, 1.76 mmol) in DCM (4 mL) and the reaction mixture was stirred at 20° C. for 3 hours. The reaction mixture was concentrated under reduced pressure to give the title compound as a white solid (430 mg) which was used without further purification.

### 2-(2,6-Dioxopiperidin-3-yl)-5-(6-(hydroxymethyl)-2-azaspiro[3.3]heptan-2-yl)isoindoline-1,3-dione

DIPEA (1.24 mL, 7.13 mmol) was added to a mixture of 2-azaspiro[3.3]heptan-6-ylmethanol (430 mg, 1.78 mmol) and 2-(2,6-dioxo-3-piperidyl)-5-fluoro-isoindoline-1,3-dione (492.42 mg, 1.78 mmol) in NMP (5 mL) and the reaction mixture was stirred at 90° C. for 16 hours. The reaction mixture was quenched with water (10 mL) and extracted with EtOAc (15 mL×3). The combined organic phases were washed with brine (25 mL), dried with anhydrous Na₂SO₄, filtered, concentrated under reduced pressure and purified by flash silica gel chromatography (ethyl acetate/petroleum ether, 30-45%) to give the title compound as a yellow oil (200 mg, 20%). ¹H NMR (400 MHz, CDCl₃) δ 9.79 (d, J=1.4 Hz, 1H), 8.06 (br s, 1H), 7.64 (d, J=8.2 Hz, 1H), 6.76 (d, J=2.0 Hz, 1H), 6.50 (dd, J=8.2, 2.1 Hz, 1H), 4.99-4.90 (m, 1H), 4.07 (s, 2H), 3.96 (s, 2H), 3.26-3.13 (m, 1H), 2.95-2.69 (m, 3H), 2.60-2.45 (m, 4H), 2.17-2.08 (m, 1H), 2.01 (s, 1H). [M+H⁺]=384.0.

### 2-(2-(2,6-Dioxopiperidin-3-yl)-1,3-dioxoisoindolin-5-yl)-2-azaspiro[3.3]heptane-6-carbaldehyde

DMP (331.88 mg, 782.48 μmol) was added to a solution of 2-(2,6-dioxo-3-piperidyl)-5-[6-(hydroxymethyl)-2-azaspiro[3.3]heptan-2-yl] isoindoline-1,3-dione (200 mg, 521.65 μmol) in DCM (2 mL) and the reaction mixture was stirred at 15° C. for 1 hour. The reaction mixture was quenched with sat. aq. NaHCO₃ (5 mL) and then extracted with DCM (15 mL×3). The combined organic phases were washed with brine (30 mL), dried with anhydrous Na₂SO₄, filtered and concentrated under reduced pressure and purified by prep-TLC (100% EtOAc) to give the title compound as a yellow solid (100 mg, 48%). [M+H+]=382.0.

### 5-(6-((4-(4-((5-Chloro-4-((2-(isopropylsulfonyl)phenyl)amino)pyrimidin-2-yl)amino)-5-methoxy-2-methylphenyl)piperidin-1-yl)methyl)-2-azaspiro[3.3]heptan-2-yl)-2-(2,6-dioxopiperidin-3-yl)isoindoline-1,3-dione

Sodium triacetoxyborohydride (83.36 mg, 393.31 μmol) was added to a solution of 2-[2-(2,6-dioxo-3-piperidyl)-1,3-dioxo-isoindolin-5-yl]-2-azaspiro [3.3]heptanes-6-carbaldehyde (50 mg, 131.10 μmol) and 5-chloro-N4-(2-isopropylsulfonylphenyl)-N2-[2-methoxy-5-methyl-4-(4-piperidyl)phenyl]pyrimidine-2,4-diamine (74.27 mg, 131.10 μmol) in DMF (2 mL) and the reaction mixture was stirred at 15° C. for 3 hours. The reaction mixture (combined with another batch at 22 mg scale) was filtered and purified by prep-HPLC (column: Waters™ Xbridge BEH C18 100*30 mm*10 μm; mobile phase: [water(10 mM NH₄HCO₃)-ACN]; B %: 55%-85%, 8 min) and further purified by prep-HPLC column: Phenomenex Luna® C18 75*30 mm*3 μm; mobile phase: [water(0.1% TFA)-ACN]; B %: 20%-60%, 8 min) to give the title compound as a yellow solid (11.2 mg, 8%). ¹H NMR: (400 MHz, DMSO-d₆) δ 11.07 (s, 1H), 9.53 (s, 1H), 9.28 (br s, 1H), 8.50 (br d, J=8.1 Hz, 1H), 8.36 (s, 1H), 8.25 (s, 1H), 7.84 (d, J=7.9 Hz, 1H), 7.69-7.56 (m, 2H), 7.47 (s, 1H), 7.34 (t, J=7.5 Hz, 1H), 6.79 (s, 1H), 6.75 (s, 1H), 6.66 (br d, J=8.4 Hz, 1H), 5.06 (dd, J=12.8, 5.3 Hz, 1H), 4.13 (s, 2H), 3.98 (s, 2H), 3.77 (s, 3H), 3.57-3.39 (m, 3H), 3.22-2.82 (m, 5H), 2.74-2.54 (m, 4H), 2.47-2.42 (m, 2H), 2.18 (s, 3H), 2.16-2.08 (m, 2H), 2.05-1.87 (m, 5H), 1.16 (d, J=6.7 Hz, 6H). [M+H+]=895.3.

### Example 14: Synthesis of 2-{[2-({4-[1-({1-[2-(2,6-dioxopiperidin-3-yl)-1,3-dioxo-2,3-dihydro-1H-isoindol-5-yl]piperidin-4-yl}methyl)piperidin-4-yl]-2-methoxy-5-methylphenyl}amino)-5-(trifluoromethyl)pyrimidin-4-yl]amino}-N-methylbenzamide (17)

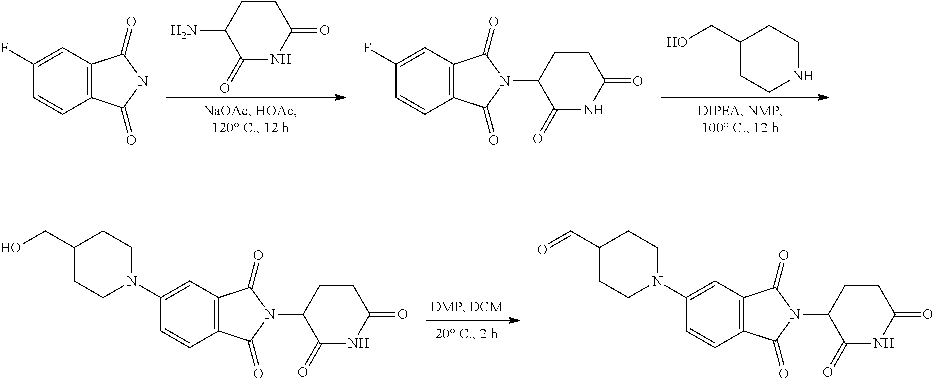

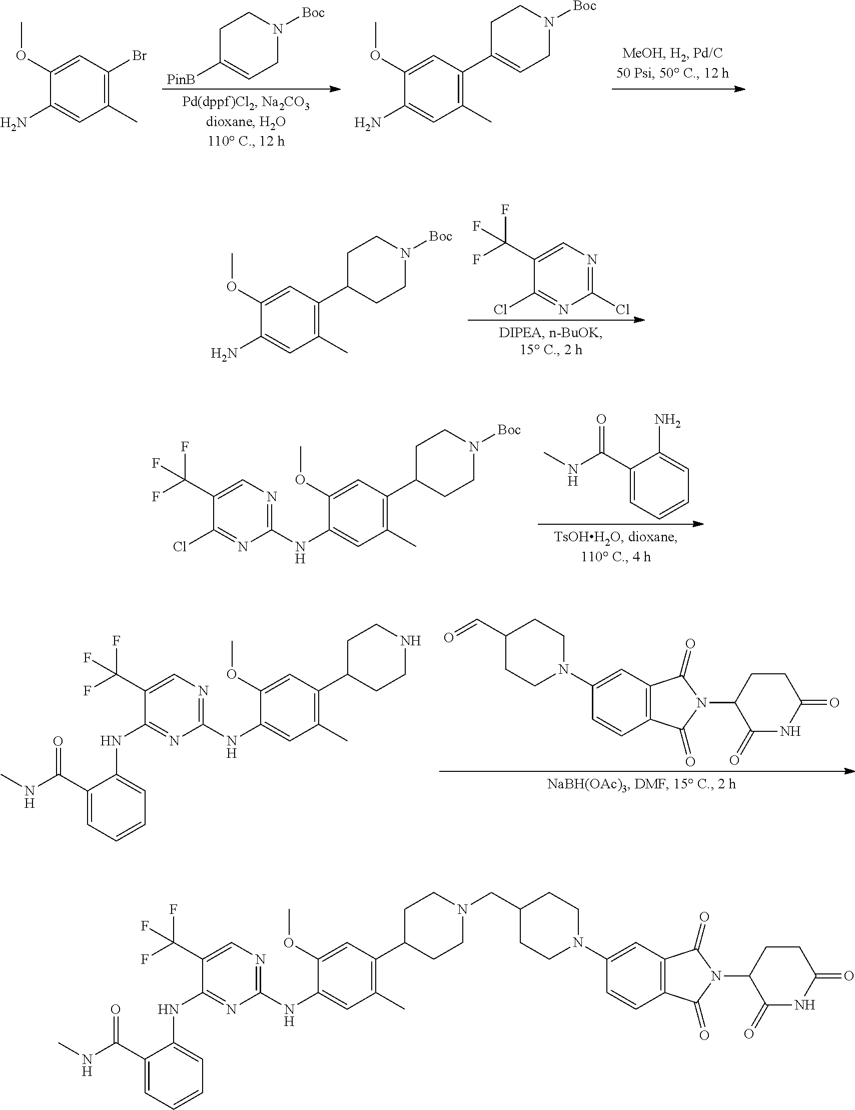

### 2-(2,6-Dioxopiperidin-3-yl)-5-fluoroisoindoline-1,3-dione

NaOAc (3.46 g, 42.14 mmol) and 3-aminopiperidine-2,6-dione (5.40 g, 42.14 mmol) were added to a solution of 5-fluoroisobenzofuran-1,3-dione (7.0 g, 42.14 mmol) in HOAc (30 mL) and the reaction mixture was stirred at 120° C. for 12 hours. The reaction mixture was concentrated under reduced pressure and diluted with H₂O (50 mL). The reaction mixture was filtered to give the title compound as a purple solid (5.5 g, 47%). ¹H NMR (400 MHz, DMSO-d₆) δ 11.14 (s, 1H), 8.04-8.00 (dd, J=8.4, 4.8 Hz, 1H), 7.87-7.85 (dd, J=7.2, 2.0 Hz, 1H), 7.73 (m, 1H), 5.20-5.15 (dd, J=13.2, 5.6 Hz, 1H), 3.32-2.87 (m, 1H), 2.64-2.51 (m, 2H), 2.10-2.07 (m, 1H).

### 2-(2,6-Dioxopiperidin-3-yl)-5-(4-(hydroxymethyl)piperidin-1-yl)isoindoline-1,3-dione

DIPEA (3.53 mL, 20.27 mmol) was added to a solution of 2-(2,6-dioxo-3-piperidyl)-5-fluoro-isoindoline-1,3-dione (1.4 g, 5.07 mmol) and 4-piperidylmethanol (583.75 mg, 5.07 mmol) in NMP (10 mL) and the reaction mixture was stirred at 100° C. for 12 hour. The reaction mixture was poured into water and stirred for 10 minutes. The aqueous phase was extracted with ethyl acetate (50 mL×2). The combined organic phases were washed with brine (150 mL), dried with anhydrous Na₂SO₄, filtered and concentrated under reduced pressure. The residue was purified by column chromatography (SiO₂, petroleum ether/ethyl acetate=100-0%) to give the title compound as a yellow solid (700 mg, 19%). [M+H⁺]=372.1.

### 1-(2-(2,6-Dioxopiperidin-3-yl)-1,3-dioxoisoindolin-5-yl)piperidine-4-carbaldehyde

DMP (428.27 mg, 1.01 mmol) was added to a mixture of 2-(2,6-dioxo-3-piperidyl)-5-[4-(hydroxymethyl)-1-piperidyl]isoindoline-1,3-dione (250 mg, 673.15 μmol) in DCM (5 mL) under N₂ and the reaction mixture was stirred at 20° C. for 2 hours. The residue was poured into sat. aq. NaHCO₃ (40 mL) and stirred for 10 minutes. The aqueous phase was extracted with DCM (40 mL×2). The combined organic phases were dried with anhydrous Na₂SO₄, filtered and concentrated under reduced pressure to give the title compound as a yellow solid (160 mg, 60%). [M+H⁺]=370.1.

### tert-Butyl 4-(4-amino-5-methoxy-2-methylphenyl)-5,6-dihydropyridine-1(2H)-carboxylate

Pd(dppf)Cl₂ (2.37 g, 3.24 mmol) and Na₂CO₃ (7.21 g, 68.03 mmol) was added to a mixture of 4-bromo-2-methoxy-5-methyl-aniline (3.5 g, 16.20 mmol) and tert-butyl 4-(4,4,5,5-tetramethyl-1,3,2-dioxaborolan-2-yl)-3,6-dihydro-2H-pyridine-1-carboxylate (13.52 g, 43.73 mmol) in dioxane (50 mL) and H₂O (10 mL) under N₂ and the reaction mixture was stirred at 110° C. for 12 hours. The reaction mixture was filtered and the filtrate was concentrated under reduced pressure. The residue was poured into water (20 mL) and the aqueous phase was extracted with ethyl acetate (30 mL×3). The combined organic phases were washed with brine (30 mL), dried with anhydrous Na₂SO₄, filtered and concentrated under reduced pressure and purified by reversed-phase HPLC to give the title compound as a yellow solid (2.5 g, 75%). ¹H NMR (400 MHz, DMSO-d₆) δ 6.57 (s, 1H), 6.43-6.39 (m, 1H), 4.40 (s, 2H), 4.07 (br d, J=11.6 Hz, 2H), 3.71 (s, 3H), 2.89-2.62 (m, 3H), 2.11 (s, 3H), 1.66-1.55 (m, 2H), 1.50-1.40 (m, 11H). [M+H⁺]=319.2.

### tert-Butyl 4-(4-amino-5-methoxy-2-methylphenyl)piperidine-1-carboxylate

Pd/C (0.5 g) was added to a solution of tert-butyl 4-(4-amino-5-methoxy-2-methyl-phenyl)-3,6-dihydro-2H-pyridine-1-carboxylate (2.5 g, 7.85 mmol) in MeOH (40 mL) under Ar. The suspension was degassed under vacuum and purged with H₂ several times. The reaction mixture was stirred under H₂ (50 psi) at 50° C. for 12 hours. The reaction mixture was filtered over Celite® and the filtrate was concentrated under reduced pressure to give the title compound as a gray solid (2.0 64%). [M+H⁺]=265.2.

### tert-Butyl 4-(4-((4-chloro-5-(trifluoromethyl)pyrimidin-2-yl)amino)-5-methoxy-2-methylphenyl)piperidine-1-carboxylate

2,4-Dichloro-5-(trifluoromethyl) pyrimidine (677.15 mg, 3.12 mmol) and DIPEA (484.02 mg, 3.75 mmol) were added to a mixture of tert-butyl 4-(4-amino-5-methoxy-2-methyl-phenyl)piperidine-1-carboxylate (1.0 g, 3.12 mmol) in n-BuOH (10 mL) under N₂ and the reaction mixture was stirred at 15° C. for 2 hours. The reaction mixture was concentrated under reduced pressure and the residue was purified by prep-HPLC (column: Phenomenex Titank C18 Bulk 250*70 mm 10 μm; mobile phase: [water(10 mM NH₄HCO₃)-ACN]; B %: 62%-92%, 20 min) to give the title compound as a white solid (380 mg, 24%).

### 2-((2-((2-Methoxy-5-methyl-4-(piperidin-4-yl)phenyl)amino)-5-(trifluoromethyl)pyrimidin-4-yl)amino)-N-methylbenzamide

TsOH·H₂O (108.22 mg, 568.93 μmol) was added to a mixture of tert-butyl 4-[4-[[4-chloro-5-(trifluoromethyl)pyrimidin-2-yl]amino]-5-methoxy-2-methyl-phenyl]piperidine-1-carboxylate (190 mg, 379.29 μmol) and 2-amino-N-methyl-benzamide (68.35 mg, 455.14 μmol) in dioxane (4 mL) under N₂ and the reaction mixture was stirred at 110° C. for 4 hours. The reaction mixture (combined with other batch at 190 mg and 20 mg scale) was concentrated under reduced pressure. The residue was purified by reversed-phase MPLC to give the title compound as a white solid (210 mg). ¹H NMR: (400 MHz, DMSO-d₆) δ 11.46 (s, 1H), 8.77-8.67 (m, 2H), 8.59 (br s, 1H), 8.46-8.21 (m, 3H), 7.69 (d, J=7.3 Hz, 1H), 7.39 (s, 1H), 7.26 (br s, 1H), 6.81 (s, 1H), 3.76 (s, 3H), 3.40 (br d, J=12.0 Hz, 2H), 3.15-2.97 (m, 6H), 2.77 (d, J=4.4 Hz, 3H), 1.96-1.79 (m, 4H). [M+H⁺]=515.2.

### 2-((2-((4-(1-((1-(2-(2,6-Dioxopiperidin-3-yl)-1,3-dioxoisoindolin-5-yl)piperidin-4-yl)methyl)piperidin-4-yl)-2-methoxy-5-methylphenyl)amino)-5-(trifluoromethyl)pyrimidin-4-yl)amino)-N-methylbenzamide

NaBH(OAc)₃ (65.90 mg, 310.96 μmol) was added to a mixture of 2-[[2-[2-methoxy-5-methyl-4-(4-piperidyl)anilino]-5-(trifluoromethyl) pyrimidin-4-yl]amino]-N-methyl-benzamide (80 mg, 155.48 μmol) and 1-[2-(2,6-dioxo-3-piperidyl)-1,3-dioxo-isoindolin-5-yl]piperidine-4-carbaldehyde (57.43 mg, 155.48 μmol) in DMF (2 mL) and the reaction mixture was stirred at 15° C. for 2 hours. The reaction mixture (combined with another batch at 30 mg scale) was purified by p-HPLC (column: Waters™ Xbridge BEH C18 100*30 mm*10 μm; mobile phase: [water(10 mM NH₄HCO₃)-ACN]; B %: 50%-80%, 8 min) to give the title compound as a white solid (57.7 mg). ¹H NMR: (400 MHz, DMSO-d₆) δ 11.45 (s, 1H), 11.09 (s, 1H), 8.77-8.63 (m, 2H), 8.36 (s, 2H), 7.72-7.63 (m, 2H), 7.37 (s, 1H), 7.32 (s, 1H), 7.24 (d, J=8.6 Hz, 2H), 7.10 (t, J=7.5 Hz, 1H), 6.90 (s, 1H), 5.08 (dd, J=12.8, 5.4 Hz, 1H), 4.06 (d, J=12.5 Hz, 2H), 3.79 (s, 3H), 3.05-2.83 (m, 5H), 2.78 (d, J=4.3 Hz, 3H), 2.72-2.53 (m, 3H), 2.24-2.15 (m, 5H), 2.02 (s, 3H), 1.91-1.63 (m, 7H), 1.25-1.11 (m, 2H). [M+H⁺]=868.2.

### Example 15: Synthesis of 2-{[2-({4-[1-({1-[2-(2,6-dioxopiperidin-3-yl)-1,3-dioxo-2,3-dihydro-1H-isoindol-5-yl]azetidin-3-yl}methyl)piperidin-4-yl]-5-methyl-2-(propan-2-yloxy)phenyl}amino)-5-(trifluoromethyl)pyrimidin-4-yl]amino}-N-methylbenzamide (18)

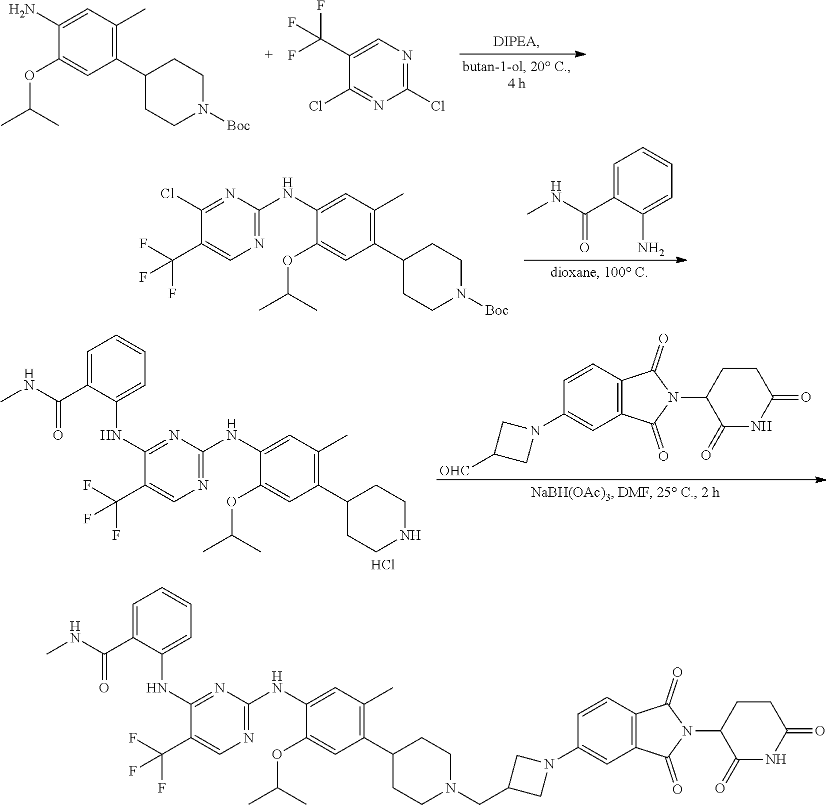

### tert-Butyl 4-(4-((4-chloro-5-(trifluoromethyl)pyrimidin-2-yl)amino)-5-isopropoxy-2-methylphenyl)piperidine-1-carboxylate

DIPEA (1.78 mg, 13.8 mmol) was added to a mixture of tert-butyl 4-(4-amino-5-isopropoxy-2-methyl-phenyl)piperidine-1-carboxylate (4.0 g, 11.48 mmol) and 2,4-dichloro-5-(trifluoromethyl)pyrimidine (2.5 g, 11.5 mmol) in n-BuOH (40 mL) and the reaction mixture was stirred at 20° C. for 4 hours. After completion of the reaction, the reaction mixture was concentrated under reduced pressure and the residue was purified by p-HPLC (column: Phenomenex Titank C18 Bulk 250*100 mm 10 μm; mobile phase: [water(10 mM NH₄HCO₃)-ACN]; B %: 80%-100%, 20 min) to give the title compound as a white solid (1.9 g, 63%). ¹H NMR (400 MHz, CDCl₃) δ 8.58 (br s, 1H), 8.17 (s, 1H), 7.98 (br s, 1H), 6.74 (s, 1H), 4.67-4.56 (m, 1H), 4.28 (br s, 2H), 2.89-2.80 (m, 3H), 2.34 (s, 3H), 1.76 (br d, J=12.5 Hz, 2H), 1.60-1.45 (m, 11H), 1.37 (d, J=6.1 Hz, 6H).

### 2-((2-((2-Isopropoxy-5-methyl-4-(piperidin-4-yl)phenyl)amino)-5-(trifluoromethyl)pyrimidin-4-yl)amino)-N-methylbenzamide

TsOH·H₂O (359.59 mg, 1.89 mmol) was added to a mixture of tert-butyl 4-[4-[[4-chloro-5-(trifluoromethyl)pyrimidin-2-yl]amino]-5-isopropoxy-2-methyl-phenyl]piperidine-1-carboxylate (1.0 g, 1.89 mmol) and 2-amino-N-methyl-benzamide (283.89 mg, 1.89 mmol) in dioxane (10 mL) and the reaction mixture was stirred at 100° C. for 12 hours. After completion of the reaction, it was cooled to 20° C. and the mixture was concentrated under reduced pressure. The crude product was purified by reversed-phase HPLC (0.1% NH₃·H₂O) to give the title compound as a white solid (300 mg, 29%).

### 2-{[2-({4-[1-({1-[2-(2,6-Dioxopiperidin-3-yl)-1,3-dioxo-2,3-dihydro-1H-isoindol-5-yl]azetidin-3-yl}methyl)piperidin-4-yl]-5-methyl-2-(propan-2-yloxy)phenyl}amino)-5-(trifluoromethyl)pyrimidin-4-yl]amino}-N-methylbenzamide

NaBH(OAc)₃ (36.60 mg, 172.70 μmol) was added to a mixture of 2-[[2-[2-isopropoxy-5-methyl-4-(4-piperidyl)anilino]-5-(trifluoromethyl) pyrimidin-4-yl]amino]-N-methyl-benzamide (50 mg, 86.35 μmol) and 1-[2-(2,6-dioxo-3-piperidyl)-1,3-dioxo-isoindolin-5-yl]azetidine-3-carbaldehyde (29.47 mg, 86.35 μmol) in DMF (1 mL) and the reaction mixture was stirred at 25° C. for 2 hours. After completion of the reaction, the mixture was concentrated under reduced pressure and the residue was purified by prep-HPLC (column: Phenomenex Gemini®-NX 80*40 mm*3 μm; mobile phase: [water (10 mM NH₄HCO₃)-ACN]; B %: 45%-75%, 8 min) to give the title compound as a yellow solid (26.7 mg). ¹H NMR (400 MHz, DMSO-d₆) δ 11.41 (s, 1H), 11.07 (s, 1H), 8.72 (br d, J=4.6 Hz, 1H), 8.59-8.22 (m, 3H), 7.69 (br d, J=6.9 Hz, 1H), 7.64 (d, J=8.3 Hz, 1H), 7.48 (s, 1H), 7.29 (br s, 1H), 7.14-7.08 (m, 1H), 6.87 (s, 1H), 6.79 (d, J=1.6 Hz, 1H), 6.65 (dd, J=8.3, 1.8 Hz, 1H), 5.05 (dd, J=12.8, 5.3 Hz, 1H), 4.58 (td, J=12.1, 6.1 Hz, 1H), 4.15 (br t, J=8.1 Hz, 2H), 3.80-3.65 (m, 2H), 3.11-2.95 (m, 3H), 2.94-2.82 (m, 1H), 2.78 (d, J=4.4 Hz, 3H), 2.71-2.57 (m, 5H), 2.24-1.95 (m, 6H), 1.68 (br s, 4H), 1.21 (d, J=5.9 Hz, 6H). ¹⁹F NMR (376 MHz, DMSO-d₆) δ-59.76 (s, 1F). [M+H⁺]=868.3.

### Example 16: Synthesis of 2-{[2-({4-[1-({1-[2-(2,6-dioxopiperidin-3-yl)-1,3-dioxo-2,3-dihydro-1H-isoindol-5-yl]piperidin-4-yl}methyl)piperidin-4-yl]-5-methyl-2-(propan-2-yloxy)phenyl}amino)-5-(trifluoromethyl)pyrimidin-4-yl]amino}-N-methylbenzamide (19)

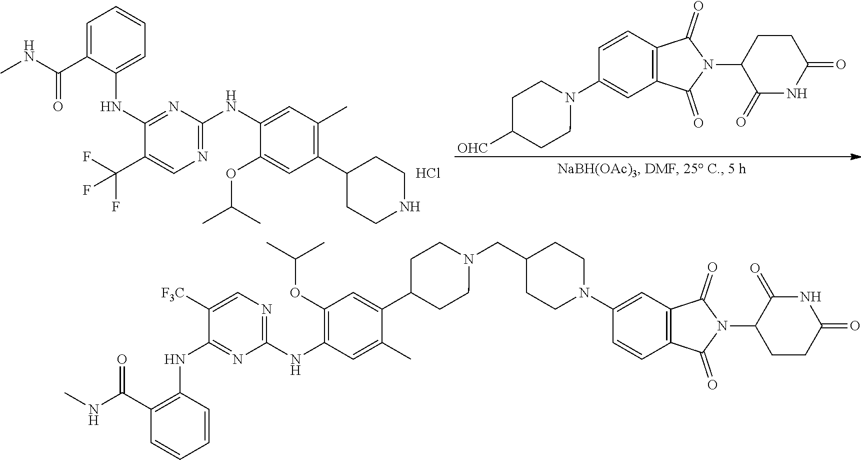

NaBH(OAc)₃ (28.18 mg, 132.98 μmol) was added to a mixture of 2-[[2-[2-isopropoxy-5-methyl-4-(4-piperidyl)anilino]-5-(trifluoromethyl)pyrimidin-4-yl]amino]-N-methyl-benzamide (75 mg, 132.98 μmol) and 1-[2-(2,6-dioxo-3-piperidyl)-1,3-dioxo-isoindolin-5-yl]piperidine-4-carbaldehyde (49.12 mg, 132.98 μmol) in DMF (3 mL) and the reaction mixture was stirred at 25° C. for 5 hours. After completion of the reaction, the reaction mixture was filtered and purified by prep-HPLC (column: Welch Xtimate® C18 100*25 mm*3 μm; mobile phase: [water (0.04% HCl)-ACN]; B %: 25%-45%, 8 min) to give the title compound as a white solid (1.9 g, 63%). ¹H NMR (400 MHz, DMSO-d₆) δ 11.80 (br s, 1H), 11.08 (s, 1H), 10.12 (br s, 1H), 9.10 (br s, 1H), 8.83 (br d, J=4.6 Hz, 1H), 8.50 (br s, 1H), 8.35 (br s, 1H), 7.75 (br d, J=7.8 Hz, 1H), 7.68 (d, J=8.4 Hz, 1H), 7.47 (s, 1H), 7.36 (s, 1H), 7.29 (br d, J=8.7 Hz, 1H), 7.17 (t, J=7.4 Hz, 1H), 7.15-7.06 (m, 1H), 6.93 (s, 1H), 5.07 (dd, J=12.8, 5.4 Hz, 1H), 4.51 (td, J=12.1, 6.1 Hz, 1H), 4.10 (br d, J=13.1 Hz, 2H), 3.61 (br d, J=11.2 Hz, 2H), 3.10-2.96 (m, 6H), 2.95-2.84 (m, 1H), 2.78 (d, J=4.4 Hz, 3H), 2.69-2.55 (m, 2H), 2.38-2.18 (m, 6H), 2.06-1.94 (m, 3H), 1.86 (br d, J=13.7 Hz, 2H), 1.57-0.64 (m, 9H). ¹⁹F NMR (377 MHz, DMSO-d₆) δ-60.31 (br s, 1F). [M+H^{+]=}896.0.

### Example 17: Synthesis of 8-(1-((1-(2-(2,6-dioxopiperidin-3-yl)-1,3-dioxoisoindolin-5-yl)azetidin-3-yl)methyl)-1H-pyrazol-4-yl)-9-ethyl-6,6-dimethyl-11-oxo-6,11-dihydro-5H-benzo[b]carbazole-3-carbonitrile (20)

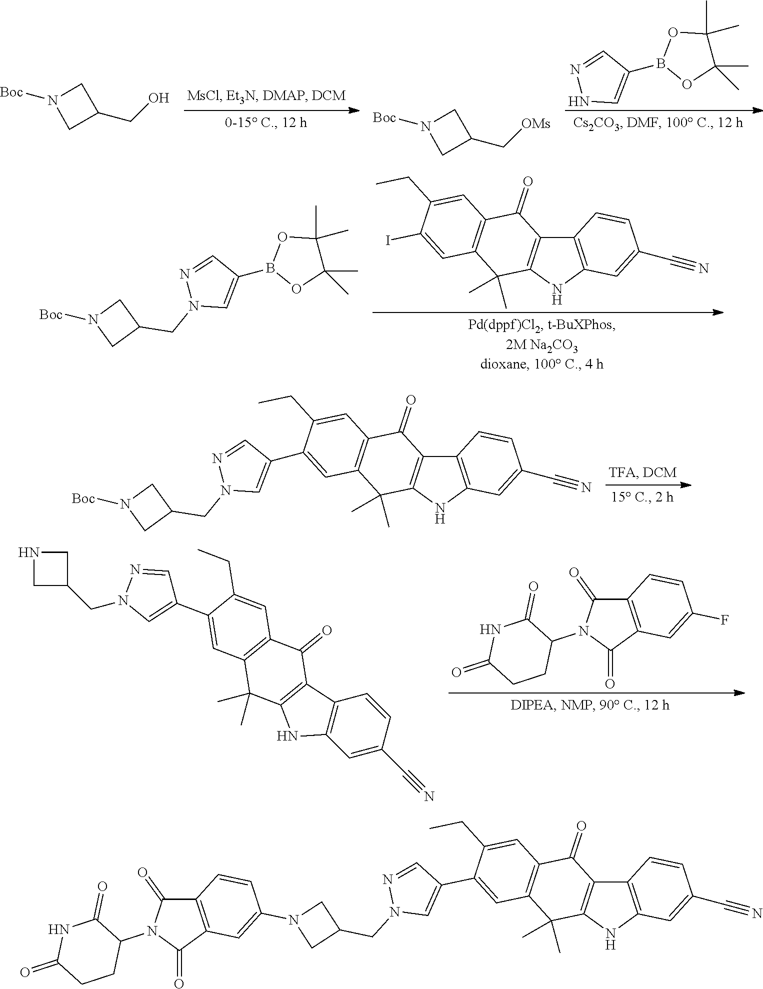

### tert-Butyl 3-(((methylsulfonyl)oxy)methyl)azetidine-1-carboxylate

DMAP (326.24 mg, 2.67 mmol) and Et₃N (5.40 g, 53.41 mmol) were added to a solution of tert-butyl 3-(hydroxymethyl)azetidine-1-carboxylate (5.0 g, 26.70 mmol) in DCM (100 mL) at 0° C. Methanesulfonyl chloride (3.36 g, 29.37 mmol) was added to the reaction mixture drop wise and the resulting mixture was allowed to warm to 15° C. The reaction mixture was stirred at 15° C. for 12 hours. The reaction mixture was quenched with sat. aq. NaHCO₃ (150 mL) and extracted with DCM (100 mL×3). The combined organic phases were washed with brine (250 mL), dried with anhydrous Na₂SO₄, filtered and concentrated under reduced pressure. The concentrate was purified by flash silica gel chromatography (ethyl acetate/petroleum ether, 50-100%) to give the title compound was a colorless oil (4.0 g, 56%). ¹H NMR (400 MHz, CDCl₃) δ 4.34 (d, J=6.8 Hz, 2H), 4.04 (t, J=8.6 Hz, 2H), 3.71 (dd, J=8.8, 5.2 Hz, 2H), 3.04 (s, 3H), 2.98-2.85 (m, 1H), 1.43 (s, 9H).

### tert-Butyl 3-((4-(4,4,5,5-tetramethyl-1,3,2-dioxaborolan-2-yl)-1H-pyrazol-1-yl)methyl) azetidine-1-carboxylate

4-(4,4,5,5-Tetramethyl-1,3,2-dioxaborolan-2-yl)-1H-pyrazole (2.19 g, 11.31 mmol) and Cs₂CO₃ (5.53 g, 16.96 mmol) were added to a solution of tert-butyl 3-(methylsulfonyloxymethyl)azetidine-1-carboxylate (3.0 g, 11.31 mmol) in DMF (125 mL) and the reaction mixture was stirred at 100° C. for 12 hours. The reaction mixture (combined with another batch at 1 g scale) was quenched with water (200 mL) and extracted with EtOAc (150 mL×3). The combined organic phases were washed with brine (350 mL), dried with anhydrous Na₂SO₄, filtered and concentrated under reduced pressure. The concentrate was purified by flash silica gel chromatography (ethyl acetate/petroleum ether, 30-50%) to give the title compound as a yellow oil (700 mg) as yellow oil. [M+H⁺]=364.2.

### tert-Butyl 3-((4-(3-cyano-9-ethyl-6,6-dimethyl-11-oxo-6,11-dihydro-5H-benzo[b]carbazol-8-yl)-1H-pyrazol-1-yl)methyl)azetidine-1-carboxylate

Na₂CO₃ (2 M, 1.75 mL) was added to a mixture of 9-ethyl-8-iodo-6,6-dimethyl-11-oxo-5H-benzo[b]carbazole-3-carbonitrile (591.23 mg, 1.34 mmol) and tert-butyl 3-[[4-(4,4,5,5-tetramethyl-1,3,2-dioxaborolan-2-yl)pyrazol-1-yl]methyl]azetidine-1-carboxylate (600 mg, 1.65 mmol) in dioxane (10 mL) and the reaction mixture was degassed with N₂. Pd(dppf)Cl₂ (58.95 mg, 80.57 μmol) and di-tert-butyl-[2-(2,4,6-triisopropylphenyl)phenyl]phosphane (21.67 mg, 51.03 μmol) were added and the reaction mixture was stirred at 100° C. for 4 hours. The reaction mixture was quenched with water (20 mL) and extracted with EtOAc (30 mL×3). The combined organic phases were washed with brine (50 mL), dried with anhydrous Na₂SO₄, filtered and concentrated under reduced pressure. The concentrate was purified by flash silica gel chromatography (ethyl acetate/petroleum ether, 80-100%) to give the title compound as a pale yellow solid (400 mg, 51%). [M+H⁺]=550.3.

### 8-(1-(Azetidin-3-ylmethyl)-1H-pyrazol-4-yl)-9-ethyl-6,6-dimethyl-11-oxo-6,11-dihydro-5H-benzo[b]carbazole-3-carbonitrile

TFA (94.29 μL, 1.27 mmol) was added to a solution of tert-butyl 3-[[4-(3-cyano-9-ethyl-6,6-dimethyl-11-oxo-5H-benzo[b]carbazol-8-yl)pyrazol-1-yl]methyl]azetidine-1-carboxylate (140 mg, 254.70 μmol) in DCM (1 mL) and the reaction mixture was stirred at 15° C. for 2 hours. The reaction mixture was concentrated under reduced pressure to give the title compound as a yellow oil (150 mg). [M+H⁺]=450.2.

### 8-(1-((1-(2-(2,6-Dioxopiperidin-3-yl)-1,3-dioxoisoindolin-5-yl)azetidin-3-yl)methyl)-1H-pyrazol-4-yl)-9-ethyl-6,6-dimethyl-11-oxo-6,11-dihydro-5H-benzo[b]carbazole-3-carbonitrile

DIPEA (386.34 μL, 2.22 mmol) was added to a solution of 8-[1-(azetidin-3-ylmethyl)pyrazol-4-yl]-9-ethyl-6,6-dimethyl-11-oxo-5H-benzo[b]carbazole-3-carbonitrile (150 mg, 266.16 μmol) and 2-(2,6-dioxo-3-piperidyl)-5-fluoro-isoindoline-1,3-dione (61.27 mg, 221.80 μmol) in NMP (2 mL) and the reaction mixture was stirred at 90° C. for 12 hours. The reaction mixture was quenched with water (8 mL) and extracted with EtOAc (15 mL×3). The combined organic phases were washed with brine (40 mL), dried with anhydrous Na₂SO₄, filtered and concentrated under reduced pressure. The concentrate was purified by prep-HPLC (column: Waters™ Xbridge BEH C18 100*25 mm*5 μm; mobile phase: [water(10 mM NH₄HCO₃)-ACN]; B %: 30%-60%, 10 min) to give the title compound as a pale yellow solid (36 mg). ¹H NMR: (400 MHz, DMSO-d₆) δ 12.75 (br s, 1H), 11.07 (br s, 1H), 8.34 (d, J=8.2 Hz, 1H), 8.21 (s, 1H), 8.10 (s, 1H), 8.02 (s, 1H), 7.85 (s, 1H), 7.78 (s, 1H), 7.68-7.60 (m, 2H), 6.81 (d, J=2.0 Hz, 1H), 6.68 (dd, J=8.4, 2.0 Hz, 1H), 5.05 (dd, J=12.8, 5.4 Hz, 1H), 4.52 (br d, J=7.0 Hz, 2H), 4.17 (t, J=8.0 Hz, 2H), 3.94 (dd, J=8.4, 5.2 Hz, 2H), 3.30 (br s, 2H), 2.81 (q, J=7.6 Hz, 2H), 2.62-2.52 (m, 2H), 2.05-1.95 (m, 1H), 1.79 (s, 6H), 1.20 (t, J=7.6 Hz, 3H). [M+H⁺]=706.1.

### Example 18: Synthesis of 3-(5-(1-(4-((5-chloro-4-((2-(isopropylsulfonyl)phenyl)amino)pyrimidin-2-yl)amino)-5-methoxy-2-methylbenzyl)-4-hydroxypiperidin-4-yl)-1-oxoisoindolin-2-yl)piperidine-2,6-dione (26)

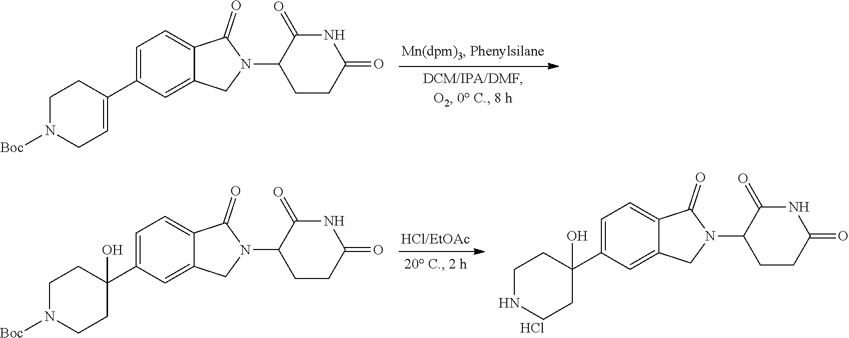 [SMILES: CC(C)(C)OC(=O)N1CC=C(c2ccc3c(c2)CN(C2CCC(=O)NC2=O)C3=O)CC1 ; CC(C)(C)OC(=O)N1CCC(O)(c2ccc3c(c2)CN(C2CCC(=O)NC2=O)C3=O)CC1 ; O=C1CCC(N2Cc3cc(C4(O)CCNCC4)ccc3C2=O)C(=O)N1 ; Cl]

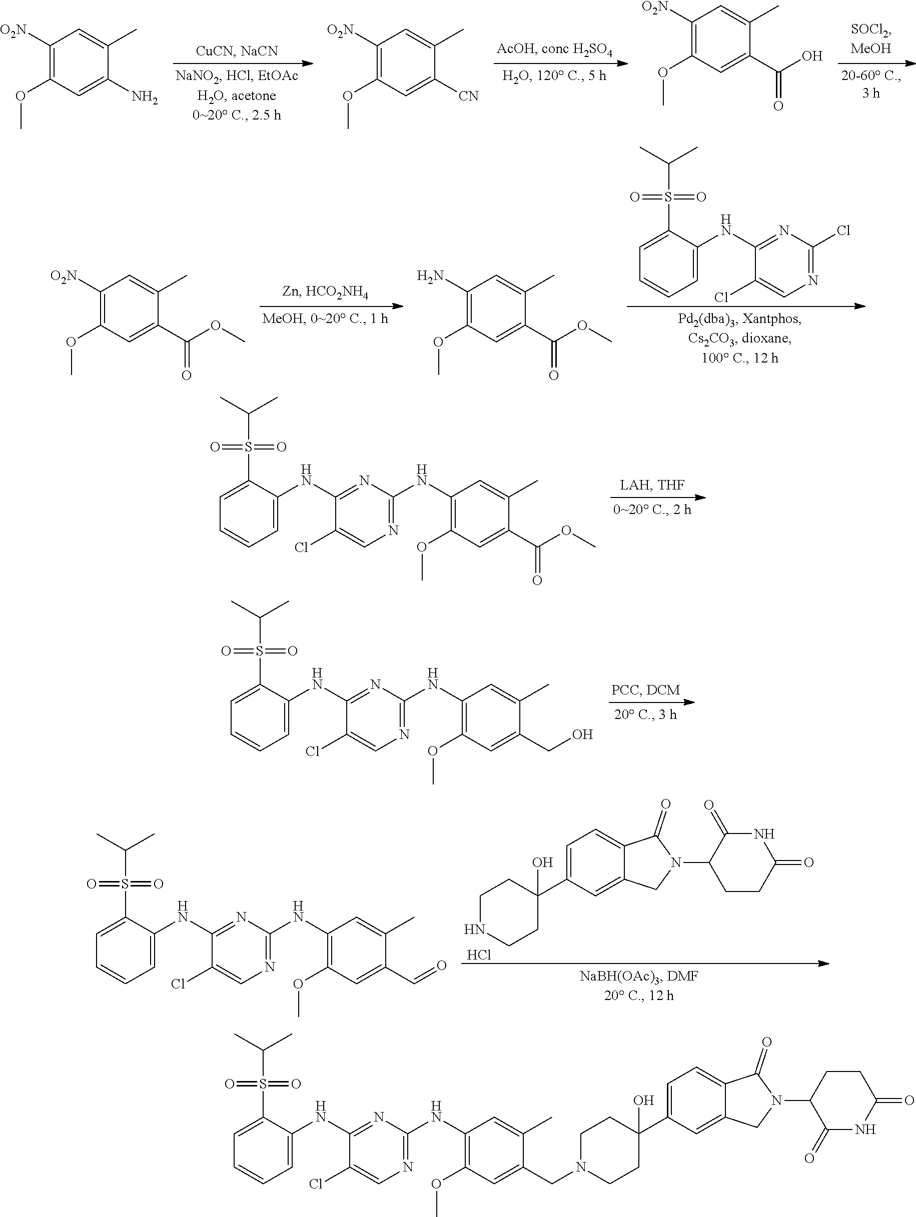

### tert-Butyl-4-(2-(2,6-dioxopiperidin-3-yl)-1-oxoisoindolin-5-yl)-4-hydroxypiperidine-1-carboxylate

Phenylsilane (508.67 mg, 4.70 mmol) was added to a solution of tert-butyl 4-[2-(2,6-dioxo-3-piperidyl)-1-oxo-isoindolin-5-yl]-3,6-dihydro-2H-pyridine-1-carboxylate (1.0 g, 2.35 mmol) and tris[(Z)-1-tert-butyl-4,4-dimethyl-3-oxo-pent-1-enoxy]manganese (710.66 mg, 1.18 mmol) in DCM (7.5 mL), IPA (7.5 mL) and DMF (1.5 mL) at 0° C. and the reaction mixture stirred for 8 hours under O₂ at 15 psi. Sat. Na₂S₂O₃ (20 mL) was added and the reaction mixture stirred at 20° C. for 2 hours. The reaction was quenched with brine (10 mL) and the reaction mixture was extracted with ethyl acetate (20 mL×3). The combined organic layers were washed with brine (50 mL), dried with anhydrous Na₂SO₄, filtered and concentrated under reduced pressure. The residue was purified by silica gel chromatography (ethyl acetate/petroleum ether, 25-100%) to give the title compound as a yellow solid (400 mg, 38%). [M+H⁺]=388.1.

### 3-(5-(4-Hydroxypiperidin-4-yl)-1-oxoisoindolin-2-yl)piperidine-2,6-dione hydrochloride

A mixture of tert-butyl 4-[2-(2,6-dioxo-3-piperidyl)-1-oxo-isoindolin-5-yl]-4-hydroxyl-piperidine-1-carboxylate (400 mg, 901.93 μmol) in HCl/dioxane (15 mL, 4 M) stirred at 20° C. for 2 hours. The mixture was concentrated under reduced pressure to give the title compound as a white solid (400 mg). [M+H⁺]=344.2.

### 5-Methoxy-2-methyl-4-nitrobenzonitrile

HCl (12 M, 2.29 mL) was added to a solution of 5-methoxy-2-methyl-4-nitro-aniline (2.0 g, 10.98 mmol) in H₂O (8 mL) and acetone (7 mL) at 0° C. under N₂. A solution of NaNO₂ (908.94 mg, 13.17 mmol) in H₂O (3 mL) was added dropwise at 0° C. and the reaction mixture stirred for 30 minutes. The reaction mixture was then added dropwise to a solution of CuCN (1.53 g, 17.13 mmol) and NaCN (2.34 g, 47.75 mmol) in H₂O (10 mL) and EtOAc (5 mL) at 0° C. The reaction then stirred at 20° C. for 2 hours. The reaction mixture was diluted with water (20 mL) and extracted with ethyl acetate (30 mL×3). The combined organic layers were washed with 2 M NaOH (40 mL), brine (50 mL), dried with anhydrous Na₂SO₄, filtered and concentrated under reduced pressure. The residue was triturated with n-hexane (20 mL) at 20° C. for 5 minutes. The mixture was filtered and the filter cake was concentrated under reduced pressure to give the title compound as a yellow solid (1.7 g, 81%). ¹H NMR: (400 MHz, CDCl₃) δ=7.72 (s, 1H), 7.30 (s, 1H), 3.98 (s, 3H), 2.55 (s, 3H)

### 5-Methoxy-2-methyl-4-nitrobenzoic acid

A mixture of 5-methoxy-2-methyl-4-nitro-benzonitrile (1.7 g, 8.85 mmol) in H₂O (16 mL), AcOH (16 mL) and H₂SO₄ (16 mL) was heated to 120° C. and stirred for 5 hours. The mixture was cooled to 20° C. and water (30 mL) was added. The reaction mixture was extracted with ethyl acetate (30 mL×2). The combined organic layers were washed with brine (30 mL), dried with anhydrous Na₂SO₄, filtered and concentrated under reduced pressure to give the title compound as a yellow solid (1.7 g). [M−H⁺]=210.0.

### Methyl 5-methoxy-2-methyl-4-nitrobenzoate

SOCl₂ (957.75 mg, 8.05 mmol) was added dropwise to a solution of 5-methoxy-2-methyl-4-nitro-benzoic acid (1.7 g, 8.05 mmol) in MeOH (18 mL) at 20° C. The reaction mixture was heated to 60° C. and stirred for 3 hours. The solvent was removed under reduced pressure and the residue was partitioned between DCM (20 mL) and sat. aq. NaHCO₃ (20 mL). The mixture was extracted with DCM (20 mL×2). The combined organic layers were washed with brine (50 mL), dried over anhydrous Na₂SO₄, filtered and concentrated under reduced pressure. The residue was purified by flash silica gel chromatography (ethyl acetate/petroleum ether, 0-20%) to give the title compound as a yellow solid (1.3 g, 70). ¹H NMR: (400 MHz, CDCl₃) δ=7.67 (s, 1H), 7.60 (s, 1H), 3.97 (s, 2H), 3.94 (s, 3H), 2.54 (s, 3H). [M+H⁺]=226.1.

### Methyl 4-amino-5-methoxy-2-methylbenzoate

Zn (3.77 g, 57.73 mmol) was added in portions to a solution of methyl 5-methoxy-2-methyl-4-nitro-benzoate (1.3 g, 5.77 mmol) in MeOH (15 mL) was added at 20° C. Ammonia-formic acid (3.64 g, 57.73 mmol) was then added at 0° C. and the reaction stirred at 20° C. for 1 hour. The mixture was filtered over Celite® and the filtrate was concentrated under reduced pressure. The residue was dissolved in a mixture of EtOAc (50 mL) and sat. NaHCO₃ (30 mL). The mixture was extracted with ethyl acetate (50 mL×2). The combined organic layers were washed with brine (50 mL), dried with anhydrous Na₂SO₄, filtered and concentrated under reduced pressure to give the title compound as a yellow solid (1.0 g, 81%). ¹H NMR: (400 MHz, CDCl₃) δ=7.43 (s, 1H), 6.53 (s, 1H), 4.32-4.03 (m, 2H), 3.88 (s, 3H), 3.85 (s, 3H), 2.50 (s, 3H). [M+H⁺]=196.1.

### Methyl-4-((5-chloro-4-((2-(isopropylsulfonyl)phenyl)amino)pyrimidin-2-yl)amino)-5-methoxy-2-methylbenzoate

Cs₂CO₃ (2.82 g, 8.66 mmol) was added to a mixture of 2,5-dichloro-N-(2-isopropylsulfonylphenyl)pyrimidin-4-amine (1.0 g, 2.89 mmol) and methyl-4-amino-5-methoxy-2-methyl-benzoate (563.83 mg, 2.89 mmol) in 1,4-dioxane (10 mL) under N₂. Xantphos (167.12 mg, 288.82 μmol) and Pd₂(dba)₃ (132.24 mg, 144.41 μmol) were added in one portion under N₂ and the reaction mixture was heated to 100° C. and stirred for 12 hours. The reaction was quenched with water (20 mL) and stirred for 10 minutes. The aqueous phase was extracted with ethyl acetate (30 mL×3). The combined organic layers were washed with brine (30 mL), dried with Na₂SO₄, filtered and concentrated under reduced pressure. The crude product was triturated with petroleum ether:ethyl acetate (15:1) at 20° C. for 30 minutes to give the title compound as a yellow solid (700 mg, 48%) as a yellow solid. [M+H⁺]=505.1.

### (4-((5-Chloro-4-((2-(isopropylsulfonyl)phenyl)amino)pyrimidin-2-yl)amino)-5-methoxy-2-methylphenyl)methanol

Lithium aluminum hydride (LAH, 112.74 mg, 2.97 mmol) was added in portions to a solution of methyl-4-((5-chloro-4-((2-(isopropylsulfonyl)phenyl)amino)pyrimidin-2-yl)amino)-5-methoxy-2-methylbenzoate (750 mg, 1.49 mmol) in THE (15 mL) at 0° C. under N₂ and the reaction mixture stirred at 20° C. for 2 hours. The reaction was quenched with water (5 mL) at 0° C., then dried with Na₂SO₄ and filtered with Celatom®. The filtrate was concentrated under reduced pressure to give the title compound as a white solid (500 mg).

### 4-((5-Chloro-4-((2-(isopropylsulfonyl)phenyl)amino)pyrimidin-2-yl)amino)-5-methoxy-2-methylbenzaldehyde

Pyridinium chlorochromate (PCC, 451.93 mg, 2.10 mmol) was added to a mixture of (4-((5-chloro-4-((2-(isopropylsulfonyl)phenyl)amino)pyrimidin-2-yl)amino)-5-methoxy-2-methylphenyl)methanol (500 mg, 1.05 mmol) in DCM (10 mL) under N₂ and the reaction mixture stirred at 20° C. for 3 hours. The reaction mixture was filtered with Celatom®/silica gel (1/1) and the filtrate was concentrated under reduced pressure to give the title compound as a white solid (400 mg). [M+H⁺]=475.2.

### 3-(5-(1-(4-((5-Chloro-4-((2-(isopropylsulfonyl)phenyl)amino)pyrimidin-2-yl)amino)-5-methoxy-2-methylbenzyl)-4-hydroxypiperidin-4-yl)-1-oxoisoindolin-2-yl)piperidine-2,6-dione

NaBH(OAc)₃ (334.67 mg, 1.58 mmol) was added to a mixture of 4-[[5-chloro-4-(2-isopropylsulfonylanilino)pyrimidin-2-yl]amino]-5-methoxy-2-methyl-benzaldehyde (150 mg, 315.82 μmol) and 3-[5-(4-hydroxyl-4-piperidyl)-1-oxo-isoindolin-2-yl]piperidine-2,6-dione (239.92 mg, 631.63 μmol) in DMF (10 mL) at 20° C. under N₂ and the reaction mixture stirred at 20° C. for 12 hours. The reaction mixture (combined with 40 mg scale reaction) was filtered and the filtrate was purified by prep-HPLC (column: Kromasil® C18 (250*50 mm*10 μm); mobile phase: [water(10 mM NH₄HCO₃)-ACN]; B %: 40%-60%, 10 min) to give the title compound as a white solid (21.9 mg). ¹H NMR: (400 MHz, DMSO-d₆) δ 9.50 (s, 1H), 8.52 (d, J=8.0 Hz, 1H), 8.33 (s, 1H), 8.24 (s, 1H), 7.82 (dd, J=7.6, 1.2 Hz, 1H), 7.73 (s, 1H), 7.71-7.64 (m, 2H), 7.60 (t, J=7.6 Hz, 1H), 7.46 (s, 1H), 7.31 (t, J=7.6 Hz, 1H), 7.00 (s, 1H), 5.11 (dd, J=13.6, 1.2 Hz, 1H), 5.05 (s, 1H), 4.44 (d, J=17.2 Hz, 1H), 4.31 (d, J=17.2 Hz, 1H), 3.78 (s, 3H), 3.49-3.39 (m, 3H), 2.92-2.85 (m, 1H), 2.68-2.58 (m, 4H), 2.45-3.38 (m, 2H), 2.22 (s, 3H), 2.01-1.96 (m, 3H), 1.63 (d, J=12.0 Hz, 2H), 1.16 (d, J=6.8 Hz, 6H). [M+H⁺]=802.2.

### Example 19: Synthesis of 5-(1-(4-((5-chloro-4-((2-(isopropylsulfonyl)phenyl)amino)pyrimidin-2-yl)amino)-5-methoxy-2-methylphenethyl)-4-hydroxypiperidin-4-yl)-2-(2,6-dioxopiperidin-3-yl)isoindoline-1,3-dione (28)

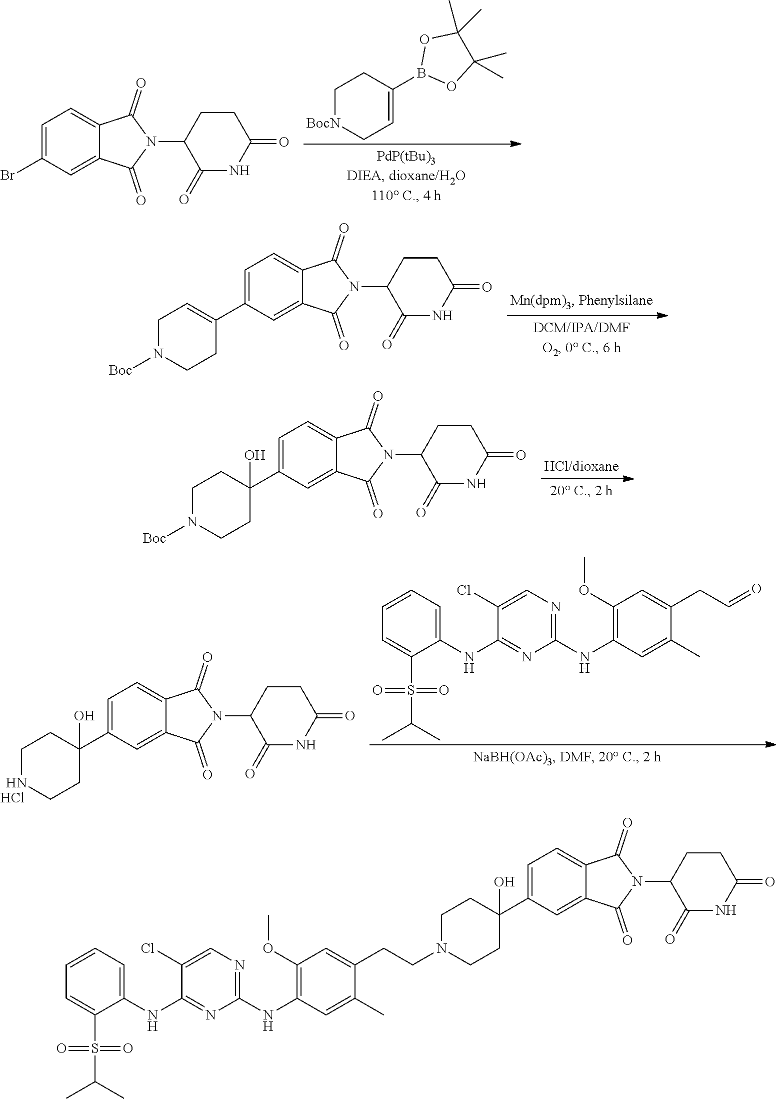

### tert-Butyl-4-(2-(2,6-dioxopiperidin-3-yl)-1,3-dioxoisoindolin-5-yl)-5,6-dihydropyridine-1-(2H)-carboxylate

DIEA (153.35 mg, 1.19 mmol) and palladium; tritert-butylphosphane (60.64 mg, 118.65 μmol) were added to a solution of 5-bromo-2-(2,6-dioxo-3-piperidyl)isoindoline-1,3-dione (200 mg, 593.25 μmol) and tert-butyl-4-(4,4,5,5-tetramethyl-1,3,2-dioxaborolan-2-yl)-3,6-dihydro-2H-pyridine-1-carboxylate (733.76 mg, 2.37 mmol) in dioxane (4 mL) and H₂O (0.2 mL) and the reaction mixture stirred at 110° C. for 4 hours under N₂. The reaction mixture was filtered through Celite® and the filtrate was concentrated under reduced pressure to give a residue. The residue was purified by flash silica gel chromatography (ethyl acetate/petroleum ether, 10-50%) to give the title compound as a yellow solid (230 mg, 84%). ¹H NMR: (400 MHz, CDCl₃) δ=8.20 (s, 1H), 7.87 (s, 1H), 7.80 (d, J=8.0 Hz, 1H), 7.75 (d, J=8.0 Hz, 1H), 6.27 (s, 1H), 5.02-4.97 (m, 1H), 4.15-4.14 (m, 2H), 3.68 (t, J=5.6 Hz, 2H), 2.94-2.76 (m, 3H), 2.57 (s, 2H), 2.17-2.15 (m, 1H), 1.51 (s, 9H). [M+H⁺]=384.0

### tert-Butyl-4-(2-(2,6-dioxopiperidin-3-yl)-1,3-dioxoisoindolin-5-yl)-4-hydroxypiperidine-1-carboxylate

Phenylsilane (113.27 mg, 1.05 mmol) was added to a solution of tert-butyl 4-(2-(2,6-dioxopiperidin-3-yl)-1,3-dioxoisoindolin-5-yl)-5,6-dihydropyridine-1(2H)-carboxylate (230 mg, 523.37 μmol) and tris[(Z)-1-tert-butyl-4,4-dimethyl-3-oxo-pent-1-enoxy]manganese (158.25 mg, 261.68 μmol) in DCM (2 mL), IPA (2 mL), and DMF (0.4 mL) under O₂ at 0° C. and the reaction mixture stirred at 0° C. for 6 hours. Sat. aq. Na₂S₂O₃ (2 mL) was added and the reaction mixture stirred at 20° C. for 2 hours. Brine (15 mL) was added and the reaction mixture was extracted with ethyl acetate (15 mL×3). The combined organic layers were dried with Na₂SO₄, filtered and concentrated under reduced pressure. The residue was purified by silica gel chromatography (ethyl acetate/petroleum ether, 20-100%) to give the title compound as a yellow solid (160 mg, 63%). ¹H NMR: (400 MHz, CDCl₃) δ=8.42 (s, 1H), 8.04-7.86 (m, 3H), 4.99 (s, 1H), 4.13-4.04 (m, 3H), 3.20 (brs, 2H), 2.80 (brs, 2H), 2.58 (brs, 1H), 2.23-1.89 (m, 3H), 1.80-1.60 (m, 2H), 1.48 (s, 9H), 1.30-1.20 (m, 1H).

### 2-(2,6-Dioxopiperidin-3-yl)-5-(4-hydroxypiperidin-4-yl)isoindoline-1,3-dione hydrochloride

A mixture of tert-butyl 4-[2-(2,6-dioxo-3-piperidyl)-1,3-dioxo-isoindolin-5-yl]-4-hydroxyl-piperidine-1-carboxylate (160 mg, 349.75 μmol) in 4 M HCl/dioxane (5 mL) stirred at 20° C. for 2 hours. The reaction mixture was concentrated under reduced pressure to give the title compound as a white solid (140 mg) as a white solid. [M+H⁺]=358.1.

### 5-(1-(4-((5-Chloro-4-((2-(isopropylsulfonyl)phenyl)amino)pyrimidin-2-yl)amino)-5-methoxy-2-methylphenethyl)-4-hydroxypiperidin-4-yl)-2-(2,6-dioxopiperidin-3-yl)isoindoline-1,3-dione

NaBH(OAc)₃ (174.24 mg, 822.12 μmol) was added to a mixture of 2-(2,6-dioxo-3-piperidyl)-5-(4-hydroxy-4-piperidyl)isoindoline-1,3-dione (107.92 mg, 274.04 μmol) and 2-[4-[[5-chloro-4-(2-isopropylsulfonylanilino)pyrimidin-2-yl]amino]-5-methoxy-2-methyl-phenyl]acetaldehyde (200 mg, 274.04 μmol) in DMF (15 mL) and the reaction mixture stirred at 20° C. for 2 hours. The reaction mixture was purified directly by prep-HPLC (Phenomenex Gemini®-NX C18 75*30 mm*3 μm; mobile phase: [water (10 mM NH₄HCO₃)-ACN]; B %: 30%-65%, 10 min) to give the title compound as a white solid (41.1 mg, 18%). ¹H NMR: (400 MHz, DMSO-d₆) δ 11.14 (s, 1H), 9.49 (s, 1H), 8.55-8.49 (m, 1H), 8.29 (s, 1H), 8.23 (s, 1H), 8.00 (t, J=4.4 Hz, 2H), 7.88 (d, J=4.1 Hz, 1H), 7.82 (d, J=7.5 Hz, 1H), 7.65-7.58 (m, 1H), 7.43 (s, 1H), 7.33 (t, J=7.0 Hz, 1H), 6.91 (s, 1H), 5.30 (s, 1H), 5.18-5.12 (m, 1H), 3.77 (s, 3H), 3.44 (td, J=13.7, 6.8 Hz, 1H), 2.97-2.74 (m, 5H), 2.68-2.54 (m, 6H), 2.16 (s, 3H), 2.10-1.98 (m, 3H), 1.66 (br d, J=11.9 Hz, 2H), 1.16 (d, J=6.8 Hz, 6H). [M+H⁺]=830.3.

### Example 20: Synthesis of 3-(5-(1-(4-((5-chloro-4-((2-(isopropylsulfonyl)phenyl)amino)pyrimidin-2-yl)amino)-5-methoxy-2-methylphenethyl)piperidin-4-yl)-1-oxoisoindolin-2-yl)piperidine-2,6-dione (27)

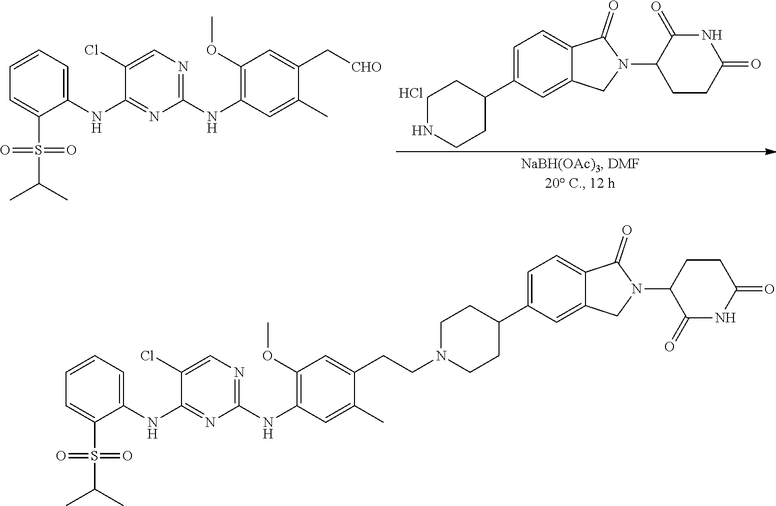 [SMILES: COc1cc(CC=O)c(C)cc1Nc1ncc(Cl)c(Nc2ccccc2S(=O)(=O)C(C)C)n1 ; Cl ; O=C1CCC(N2Cc3cc(C4CCNCC4)ccc3C2=O)C(=O)N1 ; COc1cc(CCN2CCC(c3ccc4c(c3)CN(C3CCC(=O)NC3=O)C4=O)CC2)c(C)cc1Nc1ncc(Cl)c(Nc2ccccc2S(=O)(=O)C(C)C)n1]

NaBH(OAc)₃ (433.43 mg, 2.05 mmol) was added to a mixture of 2-[4-[[5-chloro-4-(2-isopropylsulfonylanilino)pyrimidin-2-yl]amino]-5-methoxy-2-methyl-phenyl]acetaldehyde (200 mg, 409.01 μmol) and 3-[1-oxo-5-(4-piperidyl)isoindolin-2-yl]piperidine-2,6-dione (148.81 mg, 409.01 μmol) in DMF (15 mL) at 20° C. under N₂ and the reaction mixture stirred at 20° C. for 16 hours. The reaction mixture was concentrated under reduced pressure. The residue was purified by prep-HPLC (Phenomenex Gemini®-NX 80*40 mm*3 μm; mobile phase: [water (10 mM NH₄HCO₃) -ACN]; B %: 40%-60%, 8 min) to give the title compound as a white solid (33.4 mg). ¹H NMR: (400 MHz, DMSO-d₆) δ=10.98 (s, 1H), 9.49 (s, 1H), 8.52 (d, J=6.8 Hz, 1H), 8.28 (s, 1H), 8.23 (s, 1H), 7.82 (d, J=8.0 Hz, 1H), 7.68-7.54 (m, 2H), 7.50 (s, 1H), 7.46-7.37 (m, 2H), 7.33 (t, J=7.8 Hz, 1H), 6.90 (s, 1H), 5.11 (dd, J=13.2, 4.4 Hz, 1H), 4.43 (d, J=17.2 Hz, 1H), 4.29 (d, J=17.2 Hz, 1H), 3.77 (s, 3H), 3.48-3.42 (m, 1H), 3.18-3.07 (m, 1H), 2.98-2.84 (m, 1H), 2.79-2.66 (m, 3H), 2.62 (s, 2H), 2.33 (s, 1H), 2.25 (d, J=7.2 Hz, 1H), 2.20-2.00 (m, 5H), 2.00 (d, J=7.6 Hz, 2H), 1.91-1.61 (m, 4H), 1.16 (d, J=6.4 Hz, 6H). [M+H⁺]=800.3.

### Example 21: Degradation Activity

TABLE 1
Degradation activity of exemplified compounds

| | BAF3 Cellular | BAF3 Cellular | BAF3 Cellular | BAF3 Cellular |
|---|---|---|---|---|
| | Degradation - | Degradation - | Degradation - | Degradation - |
| | HiBiT Wild type | HiBiT Wild type | HiBiT G1202R | HiBiT G1202R |
| | Variant 1 | Variant 3 | Variant 1 | Variant 3 |
| | EML4-ALK: DC₅₀ | EML4-ALK: DC₅₀ | EML4-ALK: DC₅₀ | EML4-ALK: DC₅₀ |
| Compound # | (nM)/Dmax (%) | (nM)/Dmax (%) | (nM)/Dmax (%) | (nM)/Dmax (%) |
| 1 | 9/84 | 8/92 | 39/82 | 24/92 |
| 2 | 2/55 | 1/85 | 10/76 | 1/90 |
| 4 | 4/77 | 3/92 | 14/86 | 4/94 |
| 6 | 50/69 | 23/91 | ND | 79/92 |
| 7 | 20/74 | 15/90 | ND | ND |
| 8 | 14/87 | ND | 124/89 | ND |
| 9 | 8/78 | 5/89 | 41/86 | 31/91 |
| 10 | 25/73 | 6/91 | ND | 18/91 |
| 11 | 5/45 | 2/61 | ND | 3/60 |
| 13 | 14/86 | 9/92 | 117/62 | 55/79 |
| 14 | 6/59 | 3/83 | ND | 122/13 |
| 15 | 23/73 | 9/84 | ND | ND |
| 16 | 14/83 | 4/94 | 79/77 | 19/97 |
| 17 | 2/85 | 2/91 | 23/77 | 4/95 |
| 18 | 22/91 | 6/92 | 71/68 | 21/94 |
| 19 | 26/89 | 6/92 | 114/55 | 25/76 |
| 21 | 7/60 | 2/84 | ND | 16/36 |
| 22 | 5/52 | 1/84 | 6/82 | 1/94 |
| 23 | 26/73 | 6/84 | 95/63 | 32/85 |
| 26 | 57/60 | 9/49 | 147/62 | 1,701/28 |
| 27 | 3/88 | 2/84 | 8/76 | 4/81 |
| 28 | ND | 15/81 | ND | 31/79 |
| 29 | 85/71 | 25/96 | ND | 60/84 |

*ND = not determined

### Generation of Ba/F3 Stably Expressing HiBiT-Tagged Wild-Type and G1202R EML4-ALK Variants

Wild-type EMVL4-ALK variant 1 (Genflank: AB274722.1) and 3b (Genflank: AB374362.1) with HiBiT tag (SEQ ID NO 1: GTGAGCGGCTGGCGGCTGTTCAAGAAGATTAGC) fused to the C-terminal were cloned into Lentiviral vectors (pTwist Lenti SFFV Puro WPRE, Twist Biosciences). Ba/F3 cells overexpressing human CRBN were infected with the viruses for 48 hours followed by mouse IL-3 starvation for 7-10 days until the cells were capable of proliferating independent of IL-3. HiBiT-tagged EML4-ALK ALK expression were validated by Nano-Glo® HiBiT blotting system (Promega™), Western blotting and Nano-Glo® lytic detection system (Promega™). G1202R EML4-ALK Lentiviral vectors were generated by Q5@ site-directed mutagenesis kit (NEB) from the wild-type vectors. Ba/F3 cells stably expressing G1202R EML4-ALK was obtained via the same procedures as wild-type counterparts.

### Profiling for EML4-ALK Degradation Activity Using HiBiT Lytic Assay

Ba/F3 cells stably expressing indicated HiBiT-tagged EML4-ALK variant 1 were seeded at 3000 cells/well in white 384-well plates and subjected to testing compounds at 0.0003-2 μM in duplicates for 4 hours. Levels of HiBiT tag were subsequently detected using Nano-Glo® lytic detection system following manufacturer's instruction. HiBiT level was equivalent to that of EML4-ALK at any point of the assay, percentage of EML4-ALK remaining was calculated by normalizing HiBiT signal compound-treated by DMSO-treated wells. Data analysis was performed using GraphPad Prism (Version 9.1.0). DC₅₀: the concentration required to bring the curve down to the halfway point between the top and bottom plateaus of the curve. Dmax: the maximal level of degradation.

### Example 22: Antiproliferation Activity

The 3-day viability assay was performed by seeding the cells at 750 cells per 50 μL volume of complete medium per well. Following the seeding of the cells, the plate was briefly spun down to let the cells settle at the bottom of the plate before treating with compounds at a dose range using d300e. Once the cells were treated, the cells were incubated at 37° C. with 5% CO₂ for 72 hours. At the end of the treatment duration, the plate was brought to root temperature to equilibrate before adding 12.5 μL of the CellTiter-Glo® reagent per well. The plate was shaken using a plate shaken at 50 rpm for 5 minutes at room temperature and then incubated for 5 minutes in the dark before the plates were read using a luminescence based plate reader.

IC₅₀ values were determined using a non-linear regression curve fit in GraphPad Prism (Version 9.1.0). Results are shown in Table 2.

TABLE 2
Antiproliferation Activity of Compound
1 and Representative ALK Inhibitors

| | BAF3 Cells | BAF3 Cells |
|---|---|---|
| | with Variant 3 | with Variant 3 |
| | EML4-ALK^{WT} | EML4-ALK^{G1202R} |
| Compound | (IC₅₀, nM) | (IC₅₀, nM) |
| 1 | 9 | 18 |
| Crizotinib | 936 | >10,0000 |
| Ceritinib | 85 | 357 |
| Alectinib | 18 | 291 |
| Brigatinib | 17 | 183 |
| Lorlatinib | 3 | 47 |

The results indicated that degrader 1 is more resilient to the clinically relevant mutant (EML4-ALK^{G1202R}) than the inhibitor drugs.

### Example 23: Mouse Oral PK Experiments

Standard oral PK studies were conducted using male C57BI/6 mice obtained from BioDuro Shanghai. A single 2 mg/kg intravenous injection (IV) or oral administration (PO) of compound 1 solution in DMSO/Solutol/20% SBECD (5:15:80) with 12 eq 1 N HCl was evaluated. Plasma concentrations of compound 1 reported at each of the 9 time points (5 min, 15 min, 30 min, 1 h, 2 h, 4 h, 6 h, 8 h and 24 h post dosing) are the average values from 3 test animals. Results are shown in Table 3.

TABLE 3
Mouse Oral PK

| | T_{1/2} | C₀ | AUCₗₐₛₜ | AUC_{Inf} | AUC_{Extr} | V_{z} | Vₛₛ | CL | MRT_{Inf} |
|---|---|---|---|---|---|---|---|---|---|
| | (hr) | (nM) | (hr*nM) | (hr*nM) | (%) | (L/kg) | (L/kg) | (mL/min/kg) | (hr) |
| IV | 4.41 | 2340 | 4967 | 5050 | 1.65 | 2.95 | 2.29 | 7.73 | 4.95 |
| | T_{1/2} | Tₘₐₓ | Cₘₐₓ | AUCₗₐₛₜ | AUC_{Inf} | AUC_{Extr} | MRT_{Inf} | AUC_{Inf}/D | F |
| | (hr) | (hr) | (nM) | (hr*nM) | (hr*nM) | (%) | (hr) | (hr*kg*nM/nmol) | % |
| PO | 5.37 | 1.67 | 658 | 6726 | 7076 | 5.08 | 8.51 | 0.605 | 28.0 |

The results indicated that compound 1 has good in vivo metabolic stability, plasma exposure, and slow clearance rate.

### Example 24: Downregulation of FER

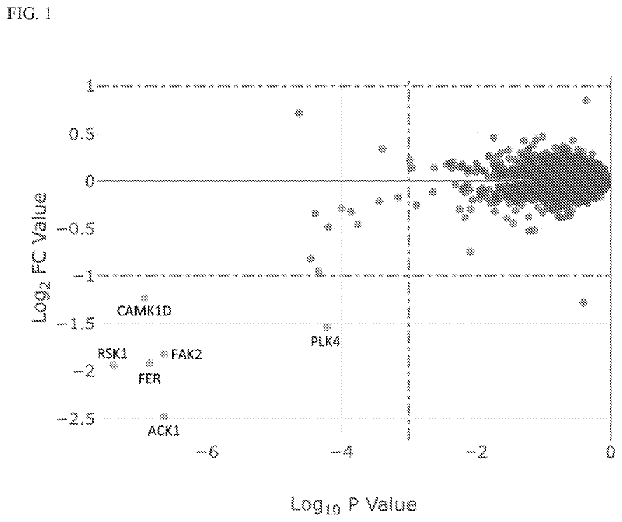

Relative fold-change (FC) abundance of proteins in MOLT4 cells treated with compound 1 (1 μM) for 5 hours was determined using quantitative mass spectrometry proteomics (protocol was published previously: Donovan et al., Cell 183:1714-1731 (2020)). Dotted lines signify a 2-fold change in protein levels and a p value of 0.001. Compound 1 downregulated PLK4, CAMK1D, FAK2, RSK1, ACK1 and FER (FIG. 1). FER is a cytoplasmic tyrosine kinase and its kinase function is not necessary for STAT3 phosphorylation in tumor cell transformation and growth, but siRNA knock down of FER leads to decreased phospho-STAT3 and reduced STAT3 activation and inhibition of tumor growth in vivo (Lennartsson et al., J. Biol. Chem. 288:15736-15744 (2013)). STAT3 activation plays a key role in several cancers, including colon cancer (Gargalionis et al., Biomedicines 9:1016 (2021)). Therefore, inhibition of STAT3 signaling through depletion of FER using a targeted protein degrader is useful in the treatment of such cancers.

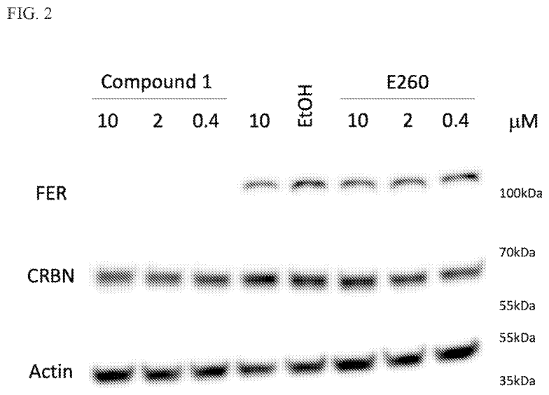

HCT116 cells were treated with compound 1 or a FER kinase inhibitor E260 (Elkis et al., Nat. Commun. 8:940 (2017)) at various concentrations for 24 hours, along with DMSO (not shown) and ethanol as controls (FIG. 2). 18 μg of protein was loaded in each lane and Western blotting was performed for FER, CRBN, and loading control ACTIN. Compound 1 significantly downregulated FER and E260 did not have any effect (FIG. 2).

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A bifunctional compound of formula (I): 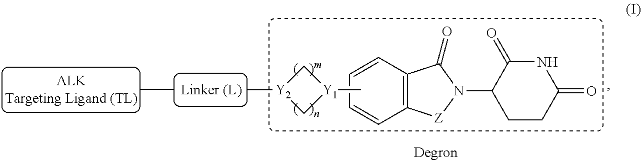
wherein:
ALK Targeting Ligand is of Formula TL-1, TL-2, or TL-3: 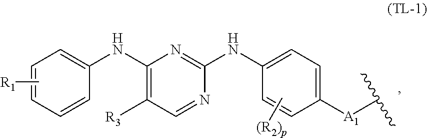
wherein:
A₁ is absent or 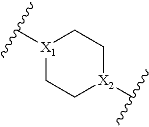
X₁ is CH or N;
X₂ is CH or N;
R₁ is —P(O)(Me)₂, —SO₂iPr, or —C(O)NHMe;
each R₂ is independently C₁-C₃ alkyl or C₁-C₃ alkoxy;
R₃ is hydrogen, halo, CN, CF₃, or C₁-C₃ alkyl; and
p is 1 or 2, or 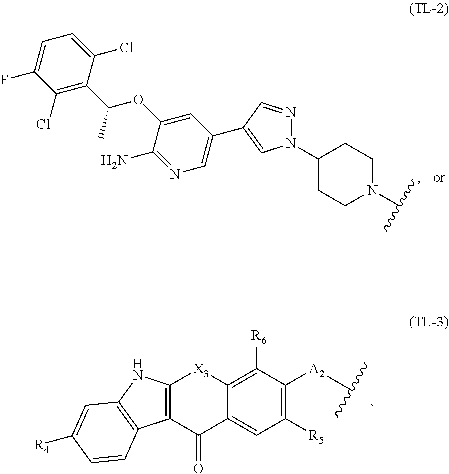
wherein:
A₂ is absent, 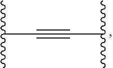 [SMILES: *C#C*]
5-membered heteroaryl, or 4- to 6-member heterocyclyl;
X₃ is CMe₂ or NR₇;
R₇ is C₁-C₈ alkyl or C₃-C₈ cycloalkyl; and
R₄, R₅, and R₆ are independently hydrogen, halo, CN, CF₃, C₁-C₃ alkyl, C₁-C₃ alkoxy, or C₃-C₆ cycloalkyl; and
Z is CH₂ or C═O;
Y₁ is N, CH, or COH;
Y₂ is N, CH, or cyclobutyl;
m is 1 or 2;
n is 1 or 2; and
the linker represents a moiety that connects covalently the degron and the targeting ligand, or a pharmaceutically acceptable salt or stereoisomer thereof.

2. The bifunctional compound of claim 1, wherein Z is CH₂.

3. The bifunctional compound of claim 1, wherein Z is C═O.

4. The bifunctional compound of claim 1, wherein Y₁ is N.

5. The bifunctional compound of claim 1, wherein Y₁ is CH.

6. The bifunctional compound of claim 1, wherein Y₁ is COH.

7. The bifunctional compound of claim 1, wherein Y₂ is N or CH.

8. (canceled)

9. The bifunctional compound of claim 1, wherein m is 1.

10. The bifunctional compound of claim 1, wherein m is 2.

11. The bifunctional compound of claim 1, wherein n is 1.

12. The bifunctional compound of claim 1, wherein n is 2.

13. The bifunctional compound of claim 1, wherein the ALK Targeting Ligand is of Formula TL-1.

14. The bifunctional compound of claim 13, wherein the ALK Targeting Ligand is of Formula TL-1a to TL-1m:

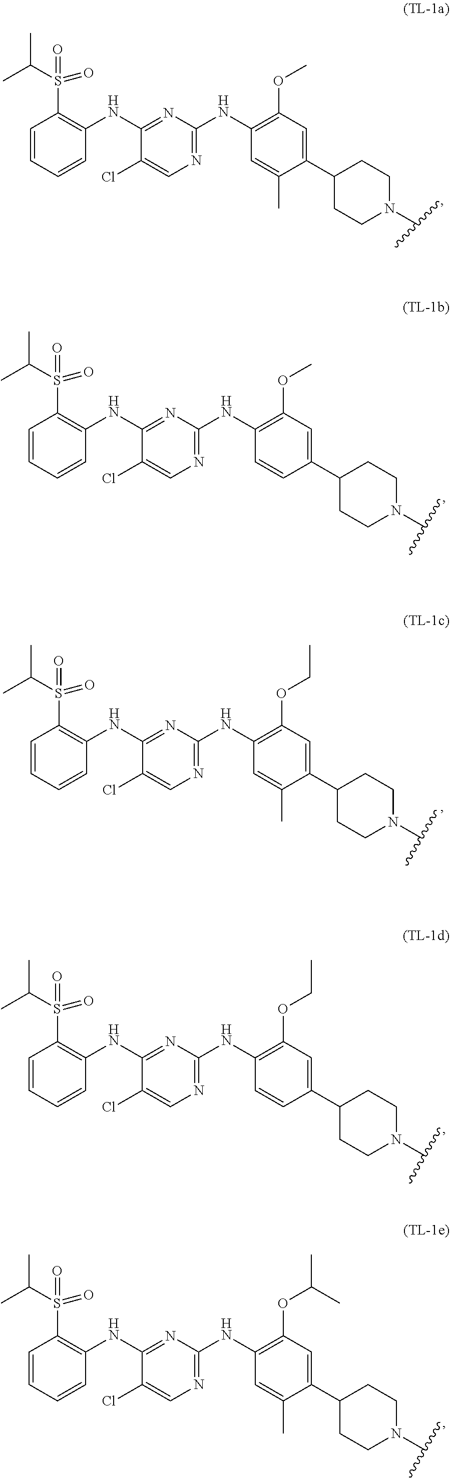 [SMILES: *N1CCC(c2cc(OC)c(Nc3ncc(Cl)c(Nc4ccccc4S(=O)(=O)C(C)C)n3)cc2C)CC1 ; *N1CCC(c2ccc(Nc3ncc(Cl)c(Nc4ccccc4S(=O)(=O)C(C)C)n3)c(OC)c2)CC1 ; *N1CCC(c2cc(OCC)c(Nc3ncc(Cl)c(Nc4ccccc4S(=O)(=O)C(C)C)n3)cc2C)CC1 ; *N1CCC(c2ccc(Nc3ncc(Cl)c(Nc4ccccc4S(=O)(=O)C(C)C)n3)c(OCC)c2)CC1 ; *N1CCC(c2cc(OC(C)C)c(Nc3ncc(Cl)c(Nc4ccccc4S(=O)(=O)C(C)C)n3)cc2C)CC1]

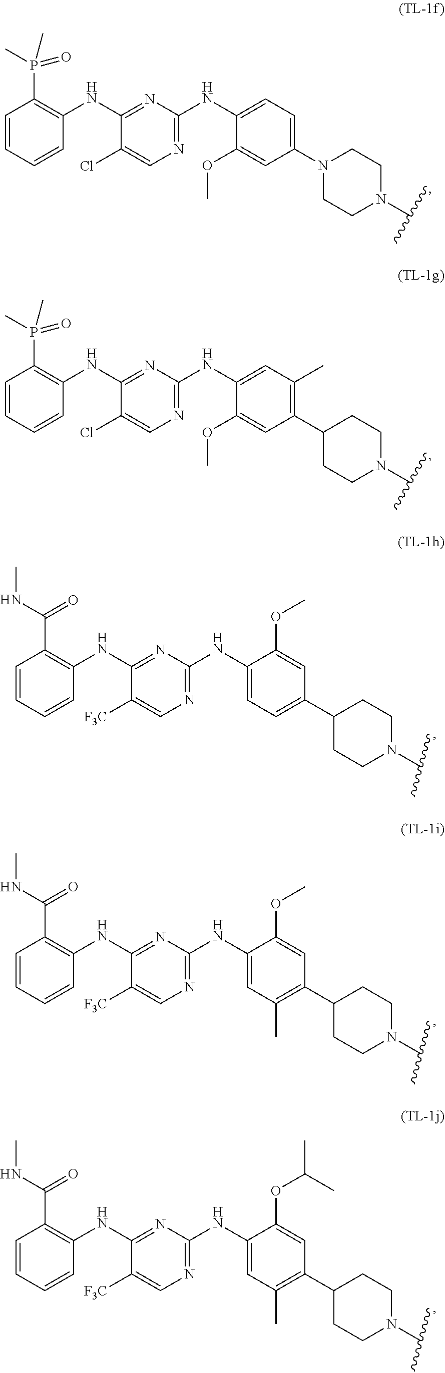 [SMILES: *N1CCN(c2ccc(Nc3ncc(Cl)c(Nc4ccccc4P(C)(C)=O)n3)c(OC)c2)CC1 ; *N1CCC(c2cc(OC)c(Nc3ncc(Cl)c(Nc4ccccc4P(C)(C)=O)n3)cc2C)CC1 ; *N1CCC(c2ccc(Nc3ncc(C(F)(F)F)c(Nc4ccccc4C(=O)NC)n3)c(OC)c2)CC1 ; *N1CCC(c2cc(OC)c(Nc3ncc(C(F)(F)F)c(Nc4ccccc4C(=O)NC)n3)cc2C)CC1 ; *N1CCC(c2cc(OC(C)C)c(Nc3ncc(C(F)(F)F)c(Nc4ccccc4C(=O)NC)n3)cc2C)CC1]

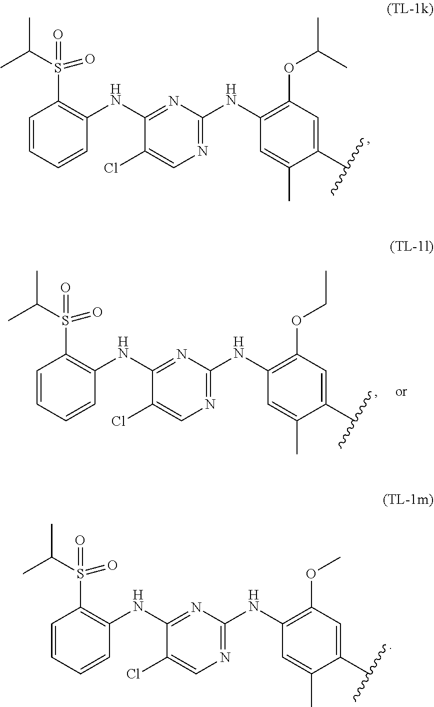

15. The bifunctional compound of claim 1, wherein the ALK Targeting Ligand is of Formula TL-2.

16. The bifunctional compound of claim 1, wherein the ALK Targeting Ligand is of Formula TL-3.

17. The bifunctional compound of claim 16, wherein the ALK Targeting Ligand is of Formula TL-3a to TL-3i:

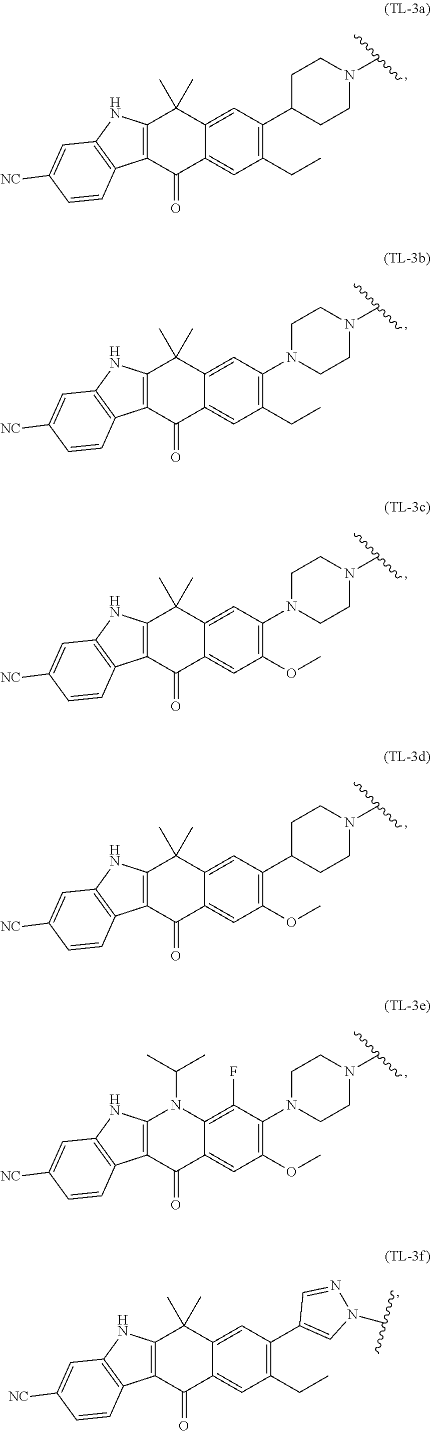 [SMILES: *N1CCC(c2cc3c(cc2CC)C(=O)c2c([nH]c4cc(C#N)ccc24)C3(C)C)CC1 ; *N1CCN(c2cc3c(cc2CC)C(=O)c2c([nH]c4cc(C#N)ccc24)C3(C)C)CC1 ; *N1CCN(c2cc3c(cc2OC)C(=O)c2c([nH]c4cc(C#N)ccc24)C3(C)C)CC1 ; *N1CCC(c2cc3c(cc2OC)C(=O)c2c([nH]c4cc(C#N)ccc24)C3(C)C)CC1 ; *N1CCN(c2c(OC)cc3c(=O)c4c5ccc(C#N)cc5[nH]c4n(C(C)C)c3c2F)CC1 ; *n1cc(-c2cc3c(cc2CC)C(=O)c2c([nH]c4cc(C#N)ccc24)C3(C)C)cn1]

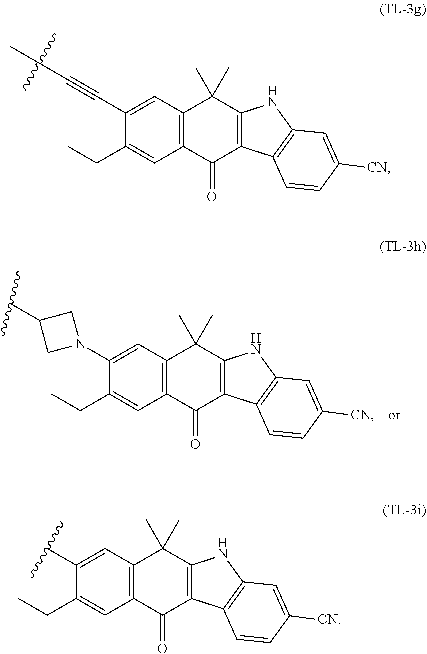 [SMILES: *C#Cc1cc2c(cc1CC)C(=O)c1c([nH]c3cc(C#N)ccc13)C2(C)C ; *C1CN(c2cc3c(cc2CC)C(=O)c2c([nH]c4cc(C#N)ccc24)C3(C)C)C1 ; *c1cc2c(cc1CC)C(=O)c1c([nH]c3cc(C#N)ccc13)C2(C)C]

18. The bifunctional compound of claim 1, wherein the Linker is of Formula L0: 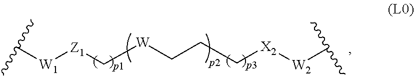
or stereoisomer thereof, wherein
p1 is an integer selected from 0 to 6;
p2 is an integer selected from 0 to 12;
p3 is an integer selected from 0 to 12;
each W is independently absent, CH₂, O, S, NR₁₀, or C(O)NH;
each R₁₀ is independently hydrogen or C₁-C₆ alkyl;
W₁ and W₂ are independently absent, (CH₂)₁₋₃, O, or NH; and
Z₁ and Z₂ are independently absent, —O—, —S—, —N(R₁₀)—, —C≡C—, —C(O)—, —C(O)O—, —OC(O)—, —OC(O)O—, —C(NOR₁₀)—, —C(O)N(R₁₀)—, —C(O)N(R₁₀)C(O)—, —C(O)N(R₁₀)C(O)N(R₁₀)—, —N(R₁₀)C(O)—, —N(R₁₀)C(O)N(R₁₀)—, —N(R₁₀)C(O)O—, —OC(O)N(R₁₀)—, —C(NR₁₀)—, —N(R₁₀)C(NR₁₀)—, —C(NR₁₀)N(R₁₀)—, —N(R₁₀)C(NR₁₀)N(R₁₀)—, —OB(Me)O—, —S(O)₂—, —OS(O)—, —S(O)O—, —S(O)—, —OS(O)₂—, —S(O)₂O—, —N(R₁₀)S(O)₂—, —S(O)₂N(R₁₀)—, —N(R₁₀)S(O)—, —S(O)N(R₁₀)—, —N(R₁₀)S(O)₂N(R₁₀)—, —N(R₁₀)S(O)N(R₁₀)—, C₃-C₁₂ carbocyclyl, or 3- to 12-membered heterocyclyl;
wherein the Linker is covalently bonded to a Degron via the 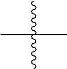
next to W₂, and covalently bonded to a Targeting Ligand via the 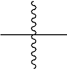
next to W₁, or the Linker is covalently bonded to a Degron via the 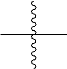
next to W₁, and covalently bonded to a Targeting Ligand via the 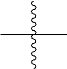
next to W₂.

19. The bifunctional compound of claim 1, wherein the linker is a bond or comprises an alkylene chain or a bivalent alkylene chain, either of which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

20. The bifunctional compound of claim 1, wherein the alkylene chain has 1-3 alkylene units, optionally substituted with C(O), or wherein the linker is a bond or represented by any one of structures:

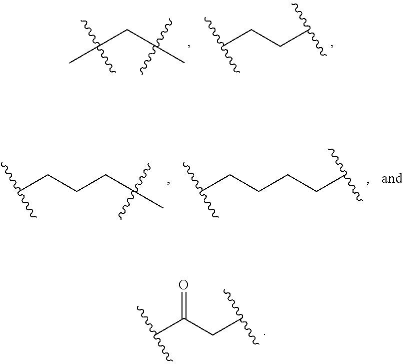

21. (canceled)

22. The bifunctional compound of claim 1, which is: 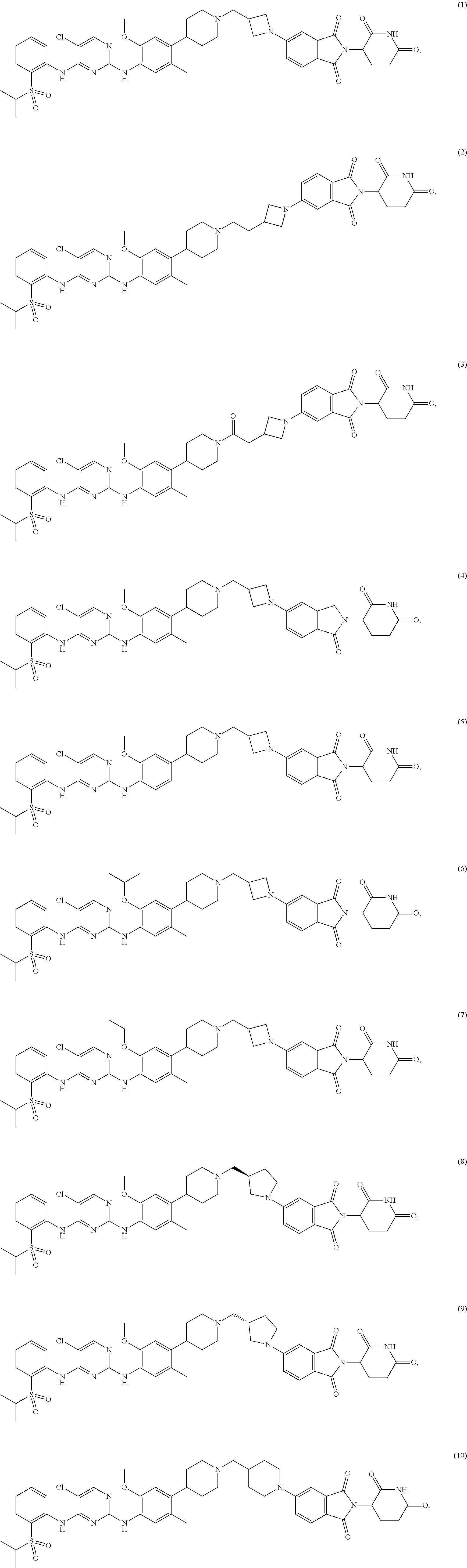 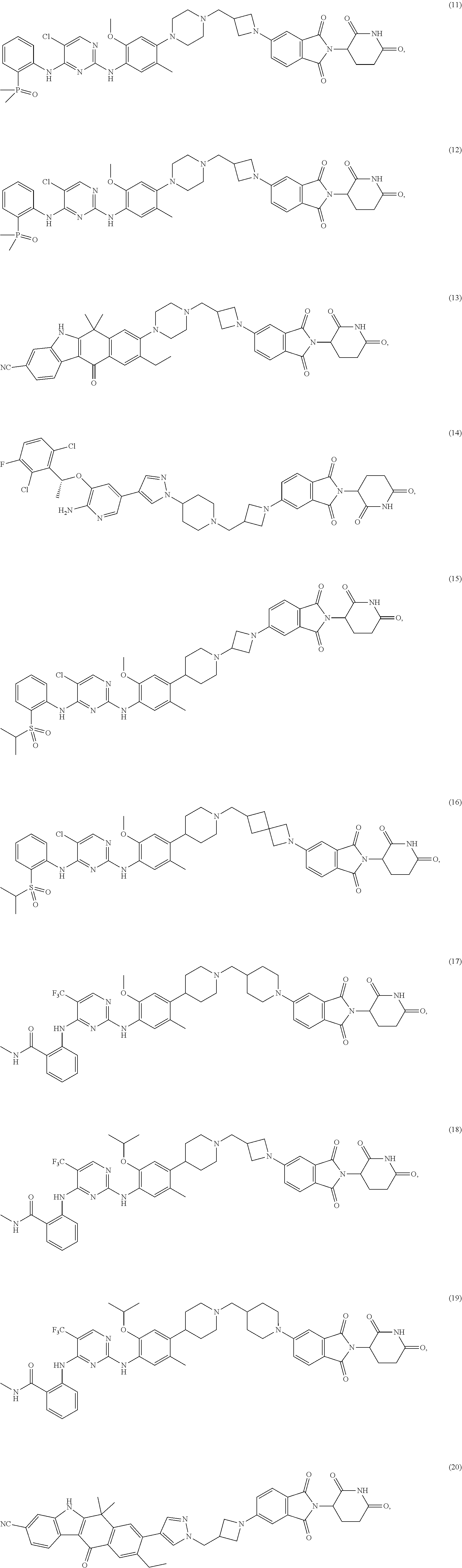 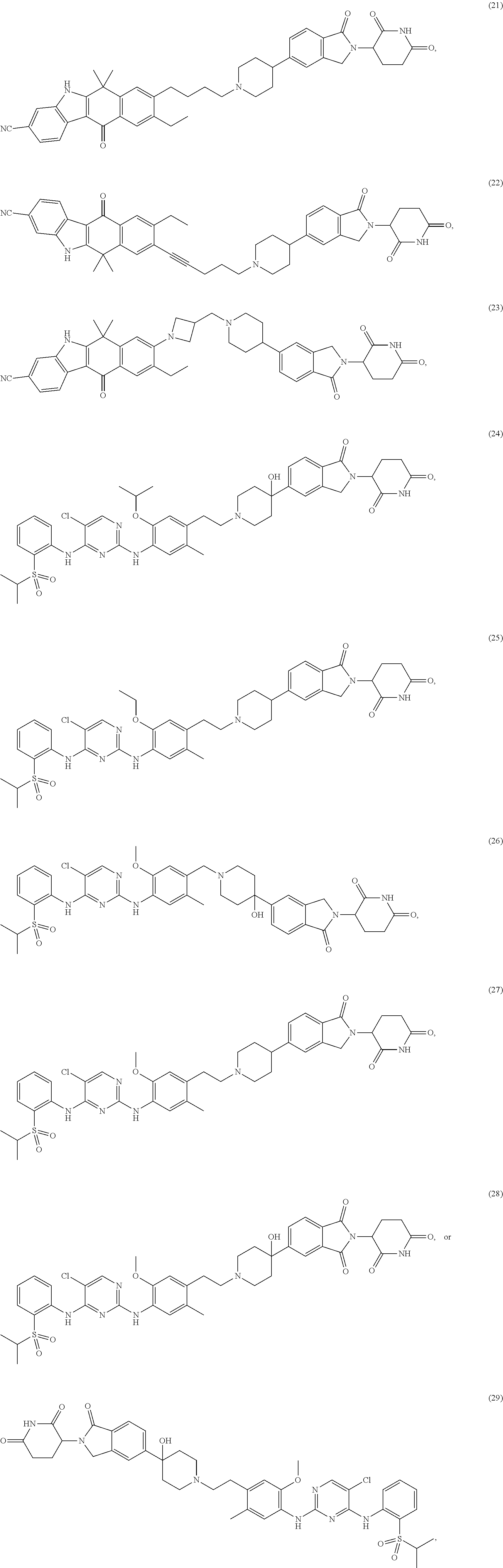
pharmaceutically acceptable salt or stereoisomer thereof.

23. A pharmaceutical composition, comprising a therapeutically effective amount of the bifunctional compound or pharmaceutically acceptable salt or stereoisomer thereof of claim 1, and a pharmaceutically acceptable carrier.

24. The pharmaceutical composition of claim 23, which is in the form of a tablet or a capsule, or which is in the form of a liquid suitable for oral or parental administration.

25. (canceled)

26. A method of treating a disease or disorder characterized or mediated by aberrant ALK activity, comprising administering a therapeutically effective amount of the bifunctional compound of claim 1 or a pharmaceutically acceptable salt or stereoisomer thereof, to a subject in need thereof.

27. The method of claim 26, wherein the disease or disorder is cancer.

28. The method of claim 27, wherein the cancer is anaplastic large cell lymphoma (ALCL), inflammatory myofibroblastic tumor (IMT), breast cancer, colorectal cancer, esophageal squamous cell cancer (ESCC), large B-cell lymphoma (DLBCL), renal cell cancer (RCC), or non-small cell lung cancer (NSCLC).
